(19) United States
(12) Patent Application Publication
(10) Pub. No.: US 2006/0288431 A1
(43) Pub. Date: Dec. 21, 2006

(54) Marker for undifferentiated state of cell and composition and method for separation and preparation of stem cells

(76) Inventors: Norio Nakatsuji, Kyoto-shi (JP); Takashi Tada, Kyoto-shi (JP); Masako Tada, Kyoto-shi (JP)

Correspondence Address: SEED INTELLECTUAL PROPERTY LAW GROUP PLLC, 701 FIFTH AVE, SUITE 5400, SEATTLE, WA, 98104, US

(21) Appl. No.: 10/545,368

(22) PCT Filed: Feb. 12, 2004
(86) PCT No.: PCT/JP04/01520

§ 371 Date: Aug. 4, 2006

(30) Foreign Application Priority Data
Feb. 13, 2003 (JP) 2003-035836
Sep. 22, 2003 (JP) 2003/330796

(51) Int. Cl. A01K 67/027; C12Q 1/68; C07H 21/04; C12P 21/06; C07K 14/705; C07K 16/28

(52) U.S. Cl. 800/8; 435/6; 435/69.1; 435/320.1; 435/325; 530/350; 530/388.22; 536/23.5

(57) ABSTRACT

A gene is provided, which can be used as a marker for determining whether a certain cell, particularly an undifferentiated cell including a tissue stem cell, has pluripotency or an undifferentiated state. The gene is called Stm and includes a Stm1 gene, which is expressed specifically in a cell under an undifferentiated state if the cell has pluripotency. A kit for determining a differentiated state of a cell is also provided. The kit comprises (a) an agent capable of reacting specifically with a Stm gene or a Stm gene product; and (b) means for determining whether or not the Stm gene is expressed in the cell.

### TECHNICAL FIELD

The present invention relates to a novel gene associated with the undifferentiated state of cells. More particularly, the present invention relates to a method for determining or controlling the undifferentiated state of cells using such a gene, a method for separating and preparing stem cells, and a composition and system associated therewith.

### BACKGROUND ART

An individual organism is formed as an aggregate of various tissue cells having a specific function. For higher organisms, all cells in each individual are originated from a single fertilized egg. Cells having pluripotency similar to that of fertilized eggs are called stem cells. The molecular mechanism for acquisition and maintenance of pluripotency is of great interest in basic biology. In addition, the application of stem cells to regenerative medicine has recently attracted attention. Stem cell research is becoming increasingly important. Identification of a gene expressed specifically in undifferentiated cells is essential for the progression of stem cell research. To date Oct3/4, UTF1, Sox1, Rex1, and the like have been reported as genes specific to undifferentiated cells. However, UTF1, Sox1, and Rex1 are also observed to be expressed in differentiated cells. Therefore, among the presently known undifferentiated cell-specific genes, only Oct3/4 can be said to be relatively strictly specific to undifferentiated cells.

Gene deletion experiments have revealed that Oct3/4 is essentially required for maintenance of an undifferentiated state. Differentiation seems to be directed depending on the expression level of the gene (Niwa, H., Miyazaki, J., and Smith, A. G. (2000), Quantitative expression of Oct3/4 defines differentiation, dedifferentiation or self-renewal of ES cells, Nat. Genet., 24, 372-376). It is expected that the mechanism for maintenance of an undifferentiated state will be clarified by identifying genes located upstream and downstream of Oct3/4. The contribution of the expression of Oct3/4 to an undifferentiated state is still unknown, however, Oct3/4 is undoubtedly an important marker gene for undifferentiated cells. An exogenous gene in which a reporter fluorescent gene (e.g., GFP (Green Fluorescence Protein) gene or the like) is placed under the control of the promoter of the Oct3/4 gene has been introduced into mice to produce transgenic mice, from which living undifferentiated cells can be purified by utilizing the expression of GFP.

As described above, there are tools for determining an undifferentiated state, such as Oct3/4 and the like. However, genes such as Oct3/4 and the like may be expressed in non-undifferentiated states. Therefore, they cannot be used as markers in the strict sense. Whereas Oct3/4 is expressed in embryonic stem cells, Oct3/4 is also expressed in unfertilized egg cells and is not expressed in other stem cells (e.g., tissue stem cells). Thus, Oct3/4 is not a perfectly accurate marker for pluripotency and its use is limited.

### DISCLOSURE OF THE INVENTION

Therefore, an object of the present invention is to provide a gene which can be used as a marker for determining whether or not a certain cell, particularly an undifferentiated cell (e.g., a tissue stem cell) has pluripotency (or an undifferentiated state).

The present invention was completed by finding Stm which is a group of genes (e.g., Stm1, etc.) which are expressed specifically in the undifferentiated state of cells which have pluripotency. It was also found that the expression of the gene is distinguishable from that of Stm2 which is a pseudogene. It was also demonstrated that Stm behaves in a fashion different from conventional markers, such as Oct3/4 and the like, at the mRNA level and at the protein level, and Stm can serve as a marker specific to a more pluripotent state, i.e., a substantially totipotent state. Stm seems to be present universally in mammalian animals, and is useful in determining mammalian animal ES cells or the like.

Therefore, the present invention provides the following.

(1) A nucleic acid molecule, comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

(2) A nucleic acid molecule according to item 1, wherein the nucleic acid molecule is at least 10 contiguous nucleotides in length.

(3) A nucleic acid molecule according to item 1, wherein the nucleic acid molecule has a sequence different from a sequence set forth in SEQ ID NO. 7 or 9 or a corresponding sequence in a corresponding nucleic acid sequence of Stm2 in at least one position in SEQ ID NO. 1, 3, 5 or 29.

(4) A nucleic acid molecule according to item 3, wherein a portion having the different sequence may be digested with a restriction enzyme.

(5) A nucleic acid molecule according to item 1, comprising a sequence set forth in SEQ ID NO. 1, 3, 5 or 29.

(6) A nucleic acid molecule, comprising:

(a) a polynucleotide having a base sequence of positions 1037 to 1607 or 244 to 1126 set forth in SEQ ID NO. 3 or a base sequence in corresponding positions, or a fragment thereof;

(b) a polynucleotide hybridizable to the polynucleotide of (a) under stringent conditions, and encoding a polypeptide biological activity; or

(c) a polynucleotide consisting of a base sequence having at least 70% identity to any one of the polynucleotides of (a) to (b) or a complementary sequence thereof, and encoding a polypeptide having biological activity.

(7) An agent, which is specific to a nucleic acid molecule according to item 1.

(8) An agent according to item 7, wherein the agent does not react specifically with a nucleic acid molecule of a Stm2 gene having a sequence set forth in SEQ ID NO. 7 or 9, or a corresponding nucleic acid sequence thereof.

(9) An agent according to item 7, wherein the agent is selected from the group consisting of a nucleic acid molecule, a polypeptide, a lipid, a sugar chain, a low molecular weight organic molecule, and a composite molecule thereof.

(10) An agent according to item 7, wherein the agent is a nucleic acid molecule of at least 8 contiguous nucleotides in length.

(11) An agent according to item 7, wherein the agent is a nucleic acid molecule and is used as a primer.

(12) An agent according to item 7, wherein the agent is used as a probe.

(13) An agent according to item 7, wherein the agent is labeled or labelable.

(14) An agent according to item 13, wherein the label is used in a technique selected from the group consisting of fluorescence, phosphorescence, chemiluminescence, radiation, enzyme-substrate reaction, and antigen-antibody reaction.

(15) A polypeptide, comprising:

(a) a polypeptide consisting of an amino acid sequence set forth in SEQ ID NO. 2, 4, 6 or 30, or a fragment thereof;

(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;

(c) a polypeptide encoded by a spliced mutant or alleic mutant of a base sequence set forth in SEQ ID NO. 1, 3, 5 or 29;

(d) a polypeptide being a species homolog of an amino acid sequence set forth in SEQ ID NO. 2, 4, 6 or 30; or

(e) a polypeptide having at least 70% identity to anyone of the polypeptides of (a) to (d) and having biological activity.

(16) A polypeptide according to item 15, wherein the polypeptide has an amino acid sequence having at least 3 contiguous amino acids.

(17) A polypeptide according to item 15, wherein the polypeptide has a sequence different from a sequence set forth in SEQ ID NO. 8 or 10 or a corresponding sequence in a corresponding amino acid sequence of Stm2 in at least one position in SEQ ID NO. 2, 4, 6 or 30.

(18) A polypeptide according to item 17, wherein a portion having the different sequence may be digested with a restriction enzyme.

(19) A polypeptide, comprising:

(a) a polypeptide consisting of an amino acid sequence of positions 157 to 218 (homeodomain), positions 261 to 301 (W-rich region), or positions 399 to 455 (B2 repeat sequence region) set forth in SEQ ID NO. 4 or an amino acid sequence in corresponding positions, or a fragment thereof;

(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;

(c) a polypeptide having at least 70% identity to anyone of the polypeptides of (a) to (b) and having biological activity.

(20) An agent, which is specific to a nucleic acid molecule according to item 15.

(21) An agent according to item 20, wherein the agent is selected from the group consisting of a nucleic acid molecule, a polypeptide, a lipid, a sugar chain, a low molecular weight organic molecule, and a composite molecule thereof.

(22) An agent according to item 20, wherein the agent is an antibody or a derivative thereof.

(23) An agent according to item 20, wherein the agent is used as a probe.

(24) An agent according to item 20, wherein the agent is labeled or labelable.

(25) An agent according to item 24, wherein the label is used in a technique selected from the group consisting of fluorescence, phosphorescence, chemiluminescence, radiation, enzyme-substrate reaction, and antigen-antibody reaction.

(26) An expression cassette, comprising a nucleic acid molecule according to item 1.

(27) A vector, comprising a nucleic acid molecule according to item 1.

(28) A vector according to item 27, further comprising a control sequence operably linked to the nucleic acid molecule.

(29) A vector according to item 28, wherein the control sequence induces expression of the nucleic acid molecule.

(30) A vector according to item 28, further comprising a sequence encoding a selectable marker.

(31) A cell, comprising a nucleic acid molecule according to item 1.

(32) A cell, comprising a nucleic acid molecule according to item 1 in a manner which allows for expression of the nucleic acid molecule.

(33) A cell, comprising a nucleic acid molecule according to item 1 in a manner which allows for expression of the nucleic acid molecule and having a desired genomic sequence.

(34) An animal tissue, comprising a nucleic acid molecule according to item 1.

(35) An animal, comprising a nucleic acid molecule according to item 1.

(36) A composition, comprising a concentrated cell comprising a nucleic acid molecule according to item 1.

(37) A nucleic acid molecule, comprising a sequence of a promoter portion of a Stm gene.

(38) A vector, comprising a nucleic acid molecule according to item 37.

(39) A vector according to item 18, further comprising a sequence encoding a selectable marker.

(40) A cell, comprising a nucleic acid molecule according to item 37.

(41) An animal tissue, comprising a nucleic acid molecule according to item 37.

(42) An animal, comprising a nucleic acid molecule according to item 37.

(43) A composition, comprising a concentrated cell comprising a nucleic acid molecule according to item 37.

(44) A composition for determining an undifferentiated state of a cell, comprising an agent capable of reacting specifically with a Stm gene or a Stm gene product.

(45) A composition according to item 44, wherein the Stm gene or Stm gene product is:

(A) a nucleic acid molecule comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity, or

(B) a polypeptide comprising:

(a) a polypeptide consisting of an amino acid sequence set forth in SEQ ID NO. 2, 4, 6 or 30, or a fragment thereof;

(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;

(c) a polypeptide encoded by a spliced mutant or alleic mutant of a base sequence set forth in SEQ ID NO. 1, 3, 5 or 29;

(d) a polypeptide being a species homolog of an amino acid sequence set forth in SEQ ID NO. 2, 4, 6 or 30; or

(e) a polypeptide having at least 70% identity to any one of the polypeptides of (a) to (d) and having biological activity.

(46) A composition according to item 44, wherein the cell is a stem cell.

(47) A composition according to item 44, wherein the cell includes an embryonic stem cell, a pluripotent stem cell, a unipotent stem cell, and a tissue stem cell.

(48) A composition according to item 44, wherein the cell includes a tissue stem cell selected from the group consisting of a neural stem cell, a gonadal stem cell, a hematopoietic stem cell, an epidermic stem cell, and mesenchymal tissue stem cell.

(49) A composition according to item 44, wherein the cell is genetically modified or is not genetically modified.

(50) A method for determining an undifferentiated state of a cell, comprising the steps of:

(I) providing a cell to be determined;

(II) contacting an agent capable of reacting specifically with a Stm gene or a Stm gene product with the cell; and

(III) detecting a specific reaction between the agent and the Stm gene or the Stm gene product to determine whether or not the Stm gene is expressed in the cell,

wherein expression of the Stm gene in the cell indicates that the cell is in an undifferentiated state.

(51) A method according to item 50, wherein the undifferentiated state is totipotency.

(52) A method according to item 50, wherein the Stm gene or the Stm gene product comprises:

(A) a nucleic acid molecule comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity, or

(B) a polypeptide comprising:

(a) a polypeptide consisting of an amino acid sequence set forth in SEQ ID NO. 2, 4, 6 or 30, or a fragment thereof;

(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;

(c) a polypeptide encoded by a spliced mutant or alleic mutant of a base sequence set forth in SEQ ID NO. 1, 3, 5 or 29;

(d) a polypeptide being a species homolog of an amino acid sequence set forth in SEQ ID NO. 2, 4, 6 or 30; or

(e) a polypeptide having at least 70% identity to any one of the polypeptides of (a) to (d) and having biological activity.

(53) A method according to item 50, further comprising determining whether or not another stem cell marker is expressed.

(54) A method according to item 53, wherein the other stem cell marker includes Oct3/4.

(55) A method according to item 50, wherein the Stm gene is a Stm1 gene.

(56) A method according to item 55, wherein the Stm1 gene comprises a sequence set forth in SEQ ID NO. 1, 3, 5 or 29.

(57) A method for preparing a cell in an undifferentiated state, comprising the steps of:

(I) providing a sample known or suspected of containing the cell in an undifferentiated state;

(II) contacting an agent capable of reacting specifically with a Stm gene or a Stm gene product with the sample;

(III) determining whether or not the Stm gene is expressed in the cell in the sample; and

(IV) isolating or concentrating the cell in which the Stm gene is expressed.

(58) A method according to item 57, wherein the undifferentiated state is totipotency.

(59) A method according to item 57, wherein the Stm gene or Stm gene product comprises:

(A) a nucleic acid molecule comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity, or

(B) a polypeptide comprising:

(a) a polypeptide consisting of an amino acid sequence set forth in SEQ ID NO. 2, 4, 6 or 30, or a fragment thereof;

(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;

(c) a polypeptide encoded by a spliced mutant or alleic mutant of a base sequence set forth in SEQ ID NO. 1, 3, 5 or 29;

(d) a polypeptide being a species homolog of an amino acid sequence set forth in SEQ ID NO. 2, 4, 6 or 30; or

(e) a polypeptide having at least 70% identity to anyone of the polypeptides of (a) to (d) and having biological activity.

(60) A method for preparing a cell in an undifferentiated state, comprising the steps of:

(I) providing the cell; and

(II) inducing expression of a Stm gene in the cell.

(61) A method according to item 60, wherein the undifferentiated state is totipotency.

(62) A method according to item 60, wherein the Stm gene comprises:

(A) a nucleic acid molecule comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

(63) A method for isolating and/or growing and/or concentrating a cell in an undifferentiated state, comprising the steps of:

(I) providing a cell;

(II) introducing a Stm gene or a Stm gene promoter into the cell; and

(III) selecting the cell in which the Stm gene or the Stm gene promoter is expressed.

(64) A method according to item 63, wherein the undifferentiated state is totipotency.

(65) A method according to item 63, wherein the Stm gene or the Stm gene promoter comprises:

(A) a nucleic acid molecule comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity, or

(B) a sequence comprising a promoter portion of a Stm1 gene.

(66) A kit for determining a differentiated state of a cell, comprising:

(a) an agent capable of reacting specifically with a Stm gene or a Stm gene product; and

(b) means for determining whether or not the Stm gene is expressed in the cell.

(67) A kit according to item 66, wherein the differentiated state is pluripotency.

(68) A kit according to item 66, wherein the differentiated state is totipotency.

(69) A kit according to item 66, wherein the Stm gene or Stm gene product comprises:

(A) a nucleic acid molecule comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity, or

(B) a polypeptide comprising:

(a) a polypeptide consisting of an amino acid sequence set forth in SEQ ID NO. 2, 4, 6 or 30, or a fragment thereof;

(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;

(c) a polypeptide encoded by a spliced mutant or alleic mutant of a base sequence set forth in SEQ ID NO. 1, 3, 5 or 29;

(d) a polypeptide being a species homolog of an amino acid sequence set forth in SEQ ID NO. 2, 4, 6 or 30; or

(e) a polypeptide having at least 70% identity to anyone of the polypeptides of (a) to (d) and having biological activity.

(70) A kit according to item 66, further comprising means for determining whether or not another stem cell marker is expressed.

(71) A kit according to item 70, wherein the other stem cell marker includes Oct3/4.

(72) A kit according to item 66, wherein the Stm gene is a Stm1 gene.

(73) A kit for preparing a cell in an undifferentiated state, comprising:

(I) an agent capable of reacting specifically with a Stm gene or a Stm gene product; and

(II) means for determining whether or not the Stm gene is expressed in the cell.

(III) isolating or concentrating the cell in which the Stm gene is expressed.

(74) A kit according to item 73, wherein the undifferentiated state is totipotency.

(75) A kit according to item 73, wherein the Stm gene or Stm gene product comprises:

(A) a nucleic acid molecule comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity, or

(B) a polypeptide comprising:

(a) a polypeptide consisting of an amino acid sequence set forth in SEQ ID NO. 2, 4, 6 or 30, or a fragment thereof;

(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;

(c) a polypeptide encoded by a spliced mutant or alleic mutant of a base sequence set forth in SEQ ID NO. 1, 3, 5 or 29;

(d) a polypeptide being a species homolog of an amino acid sequence set forth in SEQ ID NO. 2, 4, 6 or 30; or

(e) a polypeptide having at least 70% identity to any one of the polypeptides of (a) to (d) and having biological activity.

(76) A kit for preparing a cell in an undifferentiated state, comprising:

(I) means for inducing expression of a Stm gene in the cell.

(77) A kit according to item 76, wherein the undifferentiated state is totipotency.

(78) A kit according to item 76, wherein the Stm gene comprises:

(A) a nucleic acid molecule comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

(79) A kit for preparing a cell in an undifferentiated state, comprising:

(I) a vector containing a Stm gene operably linked to a control sequence.

(80) A kit according to item 79, wherein the undifferentiated state is totipotency.

(81) A kit according to item 79, wherein the Stm gene comprises:

(A) a nucleic acid molecule comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

(82) A kit for isolating and/or growing and/or concentrating a cell in an undifferentiated state, comprising:

(I) a Stm gene or a Stm gene promoter;

(II) means for introducing the Stm gene or the Stm gene promoter into the cell; and

(III) means for selecting the cell in which the Stm gene or the Stm gene promoter is expressed.

(83) A kit according to item 82, wherein the undifferentiated state is totipotency.

(84) A kit according to item 82, wherein the Stm gene comprises:

(A) a nucleic acid molecule comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity, or

(B) a sequence of a promoter portion of the Stm gene.

Hereinafter, the present invention will be described by way of preferred embodiments. It will be understood by those skilled in the art that the embodiments of the present invention can be appropriately made or carried out based on the description of the present specification and the accompanying drawings, and commonly used techniques well known in the art. The function and effect of the present invention can be easily recognized by those skilled in the art.

According to the present invention, determination of an undifferentiated state, detailed determination of totipotency or pluripotency, and the like, can be achieved which cannot be achieved with conventional agents. Thus, stem cells can be accurately determined. Further, the method of the present invention can be used to efficiently purify stem cells, such as ES cells, embryo cells, and the like.

### BRIEF DESCRIPTION OF THE DRAWINGS

FIG. 1A schematically shows mouse Stm cDNA.

FIG. 1B shows the result of Northern blot hybridization analysis on the expression pattern of Stm in embryonic stem cells, EG cells, and 12.5-day-old embryos (E12.5).

FIG. 1C shows RT-PCR analysis on Stm using total RNA recovered from adult tissues.

FIG. 1D shows the result of an experiment on forced expression of a myc-tagged Stm construct in embryonic stem cells. Ab(+) indicates a phogograph in the presence of only antibodes. Ab(+)&DAPI indicate a photograph in the presence of antibodies and DAPI. Ab(+)&Actin indicate a photograph in the presence of antibodies and actin. Ab(+)&DAPI/myc-vector indicate a photograph in the presence of antibodies and DAPI where only a myc vector was used.

FIG. 2A shows a structure of primers F2-R2 sandwiching the homeodomain of Stm. The panel in the lower portion of FIG. 2A shows RT-PCR analysis on Stm in embryos immediately after implantation to immediately before birth.

FIG. 2B shows RT-PCR analysis on Stm in unfertilized eggs, morulae, and blastocysts.

FIG. 2C (left) shows analysis on the female and male gonads of E12.5-day-old embryos. RNA was extracted from female and male gonads containing germ cells of mouse E12.5-day-old embryos. RT-PCR was performed to confirm expression of Stm1. Oct3/4 was a control for undifferentiated cells, and G3pdh was a control for RNA amount. FIG. 2C (right) shows expression of a Stm1 gene in primordial germ cells purified from E12.5-day-old gonads. 95% or more of the cells were SSEA1 positive cells, meaning that primordial germ cells were purified. In these cells, a Stm1 gene as well as a positive control Oct3/4 was expressed as shown by RT-PCR analysis. In these primordial germ cells, expression of Stm was positive as well as Oct3/4. FIG. 2C (right) shows color development with DAPI.

FIG. 2D shows expression of Stm1 and Stm2 in ES cells, E7.5 embryos, E12.5 embryos, and blastocysts.

FIG. 2E shows the transition of expression of Stm1, using antibodies. FIG. 2E shows the result of Western blot analysis on expression of a STM1 protein in undifferentiated cells.

FIG. 2F shows the transition of expression of Stm1 in cells, using antibodies.

FIG. 2G (upper column) shows comparison in the transition of expression in cells between Stm1 and Oct3/4, using antibodies. Expression was observed in a mouse, a monkey, and a human. FIG. 2G (middle column) shows the result of Stm antibody staining of a sample containing both mouse ES cells and lymphocytes. FIG. 2G (lower column) shows localization of a Stm gene in the nuclei of undifferentiated cells.

FIG. 2H shows a detailed analysis of localization of a STM1 protein in the development of mouse early embryos, using STM1 antibodies. A STM1 protein was expressed in mouse early embryos. In morulae, the nuclei of all blastomeres were positively stained. In blastocysts immediately before implantation, the nuclei of pluripotent cells, which will form an embryo, and cells in the inner cell mass were strongly stained. On the other hand, trophectoderms (cells surrouding the outer portion) were not stained, which are destined to become extraembryonic tissue including placenta. Expression of the STM1 protein was not observed in unfertilized eggs, the 8-cell stage, and the 16-cell stage.

FIG. 2I shows a detailed analysis of localization of a STM1 protein in the development of mouse early embryos, using STM1 antibodies (from E6.5 to E9.5). In 6.5-day-old embryos, 3 days after implantation, epiblasts forming the embryos were positively stained. Particularly, a border region with the extraembryonic ectoderm was strongly stained. In 7.5-day-old embryos, expression of a STM1 protein was strongly observed in a primitive streak (tail) of epiblasts. In 9.5-day-old embryos, the expression was reduced.

FIG. 2J shows the follow-up (11.5-day-old embryo) of the detailed analysis of localization of the STM1 protein in the development of mouse early embryos. The upper portion shows the whole organism, while the lower portion shows the expression in cells by staining Stm1, GFP, Stm1+GFP, and DAPI. As can be seen, expression of Stm1 was observed.

FIG. 2K shows the follow-up (13.5-day-old embryo and 16.5-day-old embryos; male and female) of the detailed analysis of localization of the STM1 protein in the development of mouse early embryos using STM1 antibodies.

FIG. 2L shows the results of detailed analysis of localization of a STM1 protein in mouse ES cells using STM1 antibodies. Stm1, Oct3/4, Stm1+Oct3/4, and DAPI show the results of staining using respective specific antibodies.

FIG. 2M shows the results of detailed analysis of localization of a STM1 protein in mouse ES cells using STM1 antibodies, which are summarized with positive and negative markers.

FIG. 2N shows the results of detailed analysis of localization of a STM1 protein in mouse cells using STM1 antibodies, where the mouse cells were stimulated with retinoic acid.

FIG. 2O shows the transition of expression of Stm1 and Oct3/4.

FIG. 3A schematically shows Stm1, Stm2, ChrX fragments, and Chr12 fragments (also referred to as Stm3 and Stm4).

FIG. 3B shows Southern blot hybridization analysis with DNA digestion by resctriction enzymes BglII and SacI.

FIG. 3C shows genome PCR analysis with a primer set of Ex3F-R2 and Lnt3F-R2.

FIG. 3D shows analysis on expression of Stm1 and Stm2 genes. F4-R4 and F3-R3 RT-PCR primers were placed on a middle portion and a 3′ portion of cDNA of Stm. It was determined whether the detected product was derived from Stm1 or Stm2, or both. All F4-R4 products were cut by digestion with a BsaMI restriction enzyme. All F3-R3 products were cut by digestion with a NlaIII restriction enzyme. Therefore, it was revealed that all of these products were derived from the Stm1 gene and that the Stm2 gene was a pseudogene.

FIG. 3E shows mapping of the Stm gene. The left portion shows a schematic diagram, while the right portion shows mouse Chromosome 7.

FIG. 4A shows a method for producing a fusion cell.

FIG. 4B shows expression of the Stm1 gene in a fusion cell of an ES cell and a somatic cell. When a thymus cell, in which expression of Stm1 is repressed, and an ES cell were fused, expression of Stm1 was detected similar to ES cells.

FIG. 4C shows expression of the somatic cell nucleus-derived Stm1 gene in an ES fusion cell. cDNA synthesized from mRNA of an ES fusion cell between subspecies (dom (Mus musculus domesticus) and mol (M.m.molossinus)) was amplified with F1-R1 primers (FIG. 1A). Somatic cell-derived products can be distinguished from ES cell-derived products based on the sensitivity to a restriction enzyme SnaBI because of base sequence polymorphisms. In both M×R (ES (mol)×Thymus (dom)) and H×J (ES (dom)×Thymus (mol)) fusion cells, expression of ES cell nucleus-derived Stm1 gene and somatic cell nucleus-derived Stm1 gene was detected.

FIG. 4D shows reactivation of the Stm1 gene due to transplantation of the nucleus of a somatic cell. The nucleus of a (M.m.molossinus(mol)×dom) F1 mouse-derived fibroblast was transplanted into a Mus musculus domesticus (dom)-derived enucleated unfertilized egg to produce a cloned blastocyst. Expression of Stm1 in the cloned blastocyst was analyzed by RT-PCR.

FIG. 4E shows re-expression of the somatic cell-derived (mol) Stm1 gene in the cloned blastocysts.

FIG. 5A shows expression of Stm1 Oct3/4, and G3pdh in cerebral stem cells (Neurosphere), embryonic stem cells (ES), thymus, and MB MAPC (pluripotent somatic stem cell). FIG. 5A shows comparison of mouse Stm1 and human Stm1 in the amino acid sequence.

FIG. 5B shows expression of Stm1 in human EC cells. Oct3/4 was a control for undifferentiated cells. G3pdh was a control for RNA.

FIG. 5C shows expression of Stm1 in mouse neural stem cells (Neurosphere) obtained from a mouse 12.5-day-old brain. Neurosphere1, 2 and 3 indicate expression of Stm1 which was obtained independently. G3pdh is a control for RNA.

FIG. 6 shows an alignment of a gene containing Stm (human, mouse and monkey).

FIG. 7 shows an alignment of the amino acid sequence of mouse Stm1 and the amino acid sequence of mouse Stm2. “*” indicates the same residue, while “.” indicates a similar residue.

FIG. 8A shows deletion constructs used when the Stm1 promoter was analyzed.

FIG. 8B shows the locations of Oct and Sox motifs in the mouse Stm1 promoter region.

FIG. 8C shows the result of experiments for identifying a promoter region in Example 15.

### DESCRIPTIN OF THE SEQUENCE LISTING

SEQ ID NOs. 1 and 2: nucleic acid and amino acid sequences of human Stm1

SEQ ID NOs. 3 and 4: nucleic acid and amino acid sequences of mouse Stm1

SEQ ID NOs. 5 and 6: nucleic acid and amino acid sequences of cynomolgus monkey Stm1

SEQ ID NOs. 7 and 8: nucleic acid and amino acid sequences of human Stm2

SEQ ID NOs. 9 and 10: nucleic acid and amino acid sequences of mouse Stm2

SEQ ID NO. 11: F1 primer

SEQ ID NO. 12: R1 primer

SEQ ID NO. 13: F2 primer

SEQ ID NO. 14: R2 primer

SEQ ID NO. 15: Oct3/4RT/1 primer

SEQ ID NO. 16: Oct3/4RT/2 primer

SEQ ID NO. 17: G3PDH-5 primer

SEQ ID NO. 18: G3PDH-3 primer

SEQ ID NO. 19: Stm-f primer

SEQ ID NO. 20: Stm-r primer

SEQ ID NO. 21: exon2F primer

SEQ ID NO. 22: exon2R primer

SEQ ID NO. 23: Ex3F primer

SEQ ID NO. 24: Lnt3F primer

SEQ ID NO. 25: F3 primer

SEQ ID NO. 26: R3 primer

SEQ ID NO. 27: F4 primer

SEQ ID NO. 28: R4 primer

SEQ ID NOs. 29 and 30: nucleic acid and amino acid sequences of rat Stm1

SEQ ID NO. 31: sequence of a promoter region of a nucleic acid sequence of human Stm1

SEQ ID NO. 32: sequence of a promoter region of a nucleic acid sequence of mouse Stm1

SEQ ID NO. 33: sequence of a promoter region of a nucleic acid sequence of cynomolgus monkey Stm1

SEQ ID NO. 34: sequence up to −2300 bp 5′ upstream of nucleic acid sequence of mouse Stm1

### BEST MODE FOR CARRYING OUT THE INVENTION

Hereinafter, the present invention will be described. It should be understood throughout the present specification that singular forms include plural referents unless the context clearly dictates otherwise. It should be also understood that the terms as used herein have definitions typically used in the art unless otherwise mentioned.

(Terms)

Terms specifically used herein will be defined below.

The term “cell” is herein used in its broadest sense in the art, referring to a structural unit of tissue of a multicellular organism, which is capable of self replicating, has genetic information and a mechanism for expressing it, and is surrounded by a membrane structure which isolates the living body from the outside. Cells used herein may be either naturally-occurring cells or artificially modified cells (e.g., fusion cells, genetically modified cells, etc.). Examples of cell sources include, but are not limited to, a single-cell culture; the embryo, blood, or body tissue of normally-grown transgenic animal; a cell mixture of cells derived from normally-grown cell lines; and the like.

As used herein, the term “stem cell” refers to a cell capable of self replication and pluripotency. Typically, stem cells can regenerate an injured tissue. Stem cells used herein may be, but are not limited to, embryonic stem. (ES) cells or tissue stem cells (also called tissular stem cell, tissue-specific stem cell, or somatic stem cell). A stem cell may be an artificially produced cell (e.g., fusion cells, reprogrammed cells, or the like used herein) as long as it can have the above-described abilities. Embryonic stem cells are pluripotent stem cells derived from early embryos. An embryonic stem cell was first established in 1981, which has been applied to production of knockout mice since 1989. In 1998, a human embryonic stem cell was established, which is currently becoming available for regenerative medicine. Tissue stem cells have a relatively limited level of differentiation unlike embryonic stem cells. Tissue stem cells are present in tissues and have an undifferentiated intracellular structure. Tissue stem cells have a higher nucleus/cytoplasm ratio and have few intracellular organelles. Most tissue stem cells have pluripotency, along cell cycle, and proliferative ability beyond the life of the individual. As used herein, stem cells may be preferably embryonic stem cells, though tissue stem cells may also be employed depending on the circumstance.

Tissue stem cells are separated into categories of sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

As used herein, the term “somatic cell” refers to any cell other than a germ cell, such as an egg, a sperm, or the like, which does not transfer its DNA to the next generation. Typically, somatic cells have limited or no pluripotency. Somatic cells used herein may be naturally-occurring or genetically modified as long as they can achieve the intended treatment.

The origin of a stem cell is categorized into the ectoderm, endoderm, or mesoderm. Stem cells of ectodermal origin are mostly present in the brain, including neural stem cells. Stem cells of endodermal origin are mostly present in bone marrow, including blood vessel stem cells, hematopoietic stem cells, mesenchymal stem cells, and the like. Stem cells of mesoderm origin are mostly present in organs, including liver stem cells, pancreas stem cells, and the like. Somatic cells may be herein derived from any germ layer. Preferably, somatic cells, such as lymphocytes, spleen cells or testis-derived cells, may be used.

As used herein, the term “isolated” means that naturally accompanying material is at least reduced, or preferably substantially completely eliminated, in normal circumstances. Therefore, the term “isolated cell” refers to a cell substantially free from other accompanying substances (e.g., other cells, proteins, nucleic acids, etc.) in natural circumstances. The term “isolated” in relation to nucleic acids or polypeptides means that, for example, the nucleic acids or the polypeptides are substantially free from cellular substances or culture media when they are produced by recombinant DNA techniques; or precursory chemical substances or other chemical substances when they are chemically synthesized. Isolated nucleic acids are preferably free from sequences naturally flanking the nucleic acid within an organism from which the nucleic acid is derived (i.e., sequences positioned at the 5′ terminus and the 3′ terminus of the nucleic acid).

As used herein, the term “established” in relation to cells refers to a state of a cell in which a particular property (pluripotency) of the cell is maintained and the cell undergoes stable proliferation under culture conditions. Therefore, established stem cells maintain pluripotency. In the present invention, the use of established stem cells is preferable since the step of collecting stem cells from a host can be avoided.

As used herein, the term “non-embryonic” refers to not being directly derived from early embryos. Therefore, the term “non-embryonic” refers to cells derived from parts of the body other than early embryos. Also, modified embryonic stem cells (e.g., genetically modified or fusion embryonic stem cells, etc.) are encompassed by non-embryonic cells.

As used herein, the term “differentiated cell” refers to a cell having a specialized function and form (e.g., muscle cells, neurons, etc.). Unlike stem cells, differentiated cells have no or little pluripotency. Examples of differentiated cells include epidermic cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells, and the like. Therefore, in one embodiment of the present invention, a cell in which a Stm gene of the present invention is expressed can acquire pluripotency even if the cell is originated from a differentiated cell.

As used herein, the terms “differentiation” or “cell differentiation” refers to a phenomenon that two or more types of cells having qualitative differences in form and/or function occur in a daughter cell population derived from the division of a single cell. Therefore, “differentiation” includes a process during which a population (family tree) of cells, which do not originally have a specific detectable feature, acquire a feature, such as production of a specific protein, or the like. At present, cell differentiation is generally considered to be a state of a cell in which a specific group of genes in the genome are expressed. Cell differentiation can be identified by searching for intracellular or extracellular agents or conditions which elicit the above-described state of gene expression. Differentiated cells are stable in principle. Particularly, animal cells which have been once differentiated are rarely differentiated into other types of cells. Therefore, the Stm gene of the present invention may be considerably useful as a marker for undifferentiated cells.

As used herein, the term “pluripotency” refers to a nature of a cell, i.e., an ability to differentiate into one or more, preferably two or more, tissues or organs. Therefore, the terms “pluripotent” and “undifferentiated” are herein used interchangeably unless otherwise mentioned. Typically, the pluripotency of a cell is limited as the cell is developed, and in an adult, cells constituting a tissue or organ rarely alter to different cells, where the pluripotency is usually lost. Particularly, epithelial cells resist altering to other type of epithelial cells. Such alteration typically occurs in pathological conditions, and is called metaplasia. However, mesenchymal cells tend to easily undergo metaplasia, i.e., alter to other mesenchymal cells, with relatively simple stimuli. Therefore, mesenchymal cells have a high level of pluripotency. Embryonic stem cells have pluripotency. Tissue stem cells have pluripotency. Thus, the term “pluripotency” may include the concept of totipotency. An example of an In vitro assay for determining whether or not a cell has pluripotency, includes, but is not limited to, culture under conditions for inducing the formation and differentiation of embryoid bodies. Examples of an in vivo assay for determining the presence or absence of pluripotency, include, but are not limited to, implantation of a cell into an immunodeficient mouse so as to form teratoma, injection of a cell into a blastocyst so as to form a chimeric embryo, implantation of a cell into a tissue of an organism (e.g., injection of a cell into ascites) so as to undergo proliferation, and the like.

As used herein, one type of pluripotency is “totipotency”, which refers to an ability to be differentiated into all kinds of cells which constitute an organism. The idea of pluripotency encompasses totipotency. An example of a totipotent cell is a fertilized ovum. Note that totipotency may be clearly separated from pluripotency. The former indicates an ability to be differentiated into all kinds of cells while the latter indicates an ability to be committed into a plurality of types of cells but not all types. An ability to be differentiated into only one type of cell is called “unipotency”.

As used herein, totipotency and pluripotency can be determined based on the number of days which has passed after fertilization. For example, for mouse, totipotency is distinguished from pluripotency with about Day 8 after fertilization as a borderline. Although not wishing to be bound by theory, for mouse, cells develop over time after fertilization as follows. On Day 6.5 after fertilization (also represented by E6.5), a primitive streak appears on the one side of an epiblast, clarifying the future anteroposterior axis of the embryo. The primitive streak indicates the future posterior end of the embryo, extending across the ectoderm to reach the distal end of the cylinder. The primitive streak is an area in which cell movement takes is place. As a result, the future endoderm and mesoderm are formed. By E7.5 a head process appears ahead of the node, in which a notochord, and a future endoderm (lower layer) and a neural plate (upper layer) around the notochord, are formed. The node appears around E6.5 and moves backward, so that the axial structure is formed from the head to the tail. By E8.5 the embryo is elongated and a large head lamella mostly consisting of the anterior neural plate is formed at the anterior end of the embryo. Segments are formed at a rate of one per 1.5 hours from E8 from the head to the tail. After this stage, cells no longer exhibit totipotency or develop into an individual even if they are brought back to the placenta, except for dedifferentiation. Before this stage, cells have totipotency without any particular treatment. Thus, this stage is a branch point of totipotency. Therefore, it is difficult to establish ES cells from embryos after this point. In other words, it is possible to establish cells, typically called EG (germ cell-derived) cells, from embryos after this point. Also, in this context this point can be said to be a branch point. Therefore, in one aspect, Stm1 of the present invention can be used to determine the presence or absence of totipotency or the validity of an ES cell as a starting material.

Cells used herein may be derived from any organism (e.g., any multicellular organism (e.g., animals (e.g., vertebrates and invertebrates), plants (e.g., monocotyledons and dicotyledons, etc.)). For example, cells used herein are derived from a vertebrate (e.g., Myxiniformes, Petronyzoniformes, Chondrichthyes, Osteichthyes, amphibian, reptilian, avian, mammalian, etc.), more preferably mammalian (e.g., monotremata, marsupialia, edentate, dermoptera, chiroptera, carnivores insectivore, proboscidea, perissodactyla, artiodactyla, tubulidentata, pholidota, sirenia, cetacean, primates, rodentia, lagomorpha, etc.). More preferably, cells derived from Primates (e.g., chimpanzee, Japanese monkey, human) are used. Most preferably, cells derived from a human are used.

Any organ may be targeted by the present invention. A tissue or cell targeted by the present invention may be derived from any organ. As used herein; the term “organ” refers to a morphologically independent structure localized at a particular portion of an individual organism in which a certain function is performed. In multicellular organisms (e.g., animals, plants), an organ consists of several tissues spatially arranged in a particular manner, each tissue being composed of a number of cells. An example of such an organ includes an organ relating to the vascular system. In one embodiment, organs targeted by the present invention include, but are not limited to, skin, blood vessel, cornea, kidney, heart, liver, umbilical cord, intestine, nerve, lung, placenta, pancreas, brain, peripheral limbs, retina, and the like. Examples of cells differentiated from pluripotent cells include epidermic cells, pancreatic parenchymal cells, pancreatic duct cells, hepatic cells, blood cells, cardiac muscle cells, skeletal muscle cells, osteoblasts, skeletal myoblasts, neurons, vascular endothelial cells, pigment cells, smooth muscle cells, fat cells, bone cells, cartilage cells, and the like.

As used herein, the term “tissue” refers to an aggregate of cells having substantially the same function and/or form in a multicellular organism. “Tissue” is typically an aggregate of cells of the same origin, but may be an aggregate of cells of different origins as long as the cells have the same function and/or form. Therefore, when stem cells of the present invention are used to regenerate tissue, the tissue may be composed of an aggregate of cells of two or more different origins. Typically, a tissue constitutes a part of an organ. Animal tissues are separated into epithelial tissue, connective tissue, muscular tissue, nervous tissue, and the like, on a morphological, functional, or developmental basis. Plant tissues are roughly separated into meristematic tissue and permanent tissue according to the developmental stage of the cells constituting the tissue. Alternatively, tissues may be separated into single tissues and composite tissues according to the type of cells constituting the tissue. Thus, tissues are separated into various categories.

The terms “protein”, “polypeptide”, “oligopeptide” and “peptide” as used herein have the same meaning and refer to an amino acid polymer having any length. This polymer may be a straight, branched or cyclic chain. An amino acid may be a naturally-occurring or nonnaturally-occurring amino acid, or a variant amino acid. The term may include those assembled into a composite of a plurality of polypeptide chains. The term also includes a naturally-occurring or artificially modified amino acid polymer. Such modification includes, for example, disulfide bond formation, glycosylation, lipidation, acetylation, phosphorylation, or any other manipulation or modification (e.g., conjugation with a labeling moiety). This definition encompasses a polypeptide containing at least one amino acid analog (e.g., nonnaturally-occurring amino acid, etc.), a peptide-like compound (e.g., peptoid), and other variants known in the art, for example.

The terms “polynucleotide”, “oligonucleotide”, and “nucleic acid” as used herein have the same meaning and refer to a nucleotide polymer having any length. This term also includes an “oligonucleotide derivative” or a “polynucleotide derivative”. An “oligonucleotide derivative” or a “polynucleotide derivative” includes a nucleotide derivative, or refers to an oligonucleotide or a polynucleotide having different linkages between nucleotides from typical linkages, which are interchangeably used. Examples of such an oligonucleotide specifically include 2′-O-methyl-ribonucleotide, an oligonucleotide derivative in which a phosphodiester bond in an oligonucleotide is converted to a phosphorothioate bond, an oligonucleotide derivative in which a phosphodiester bond in an oligonucleotide is converted to a N3′-P5′ phosphoroamidate bond, an oligonucleotide derivative in which a ribose and a phosphodiester bond in an oligonucleotide are converted to a peptide-nucleic acid bond, an oligonucleotide derivative in which uracil in an oligonucleotide is substituted with C-5 propynyl uracil, an oligonucleotide derivative in which uracil in an oligonucleotide is substituted with C-5 thiazole uracil, an oligonucleotide derivative in which cytosine in an oligonucleotide is substituted with C-5 propynyl cytosine, an oligonucleotide derivative in which cytosine in an oligonucleotide is substituted with phenoxazine-modified cytosine, an oligonucleotide derivative in which ribose in DNA is substituted with 2′-O-propyl ribose, and an oligonucleotide derivative in which ribose in an oligonucleotide is substituted with 2′-methoxyethoxy ribose. Unless otherwise indicated, a particular nucleic acid sequence also implicitly encompasses conservatively-modified variants thereof (e.g. degenerate codon substitutions) and complementary sequences as well as the sequence explicitly indicated. Specifically, degenerate codon substitutions may be produced by generating sequences in which the third position of one or more selected (or all) codons is substituted with mixed-base and/or deoxyinosine residues (Batzer et al., Nucleic Acid Res. 19:5081(1991); Ohtsuka et al., J. Biol. Chem. 260:2605-2608 (1985); Rossolini et al., Mol. Cell. Probes 8:91-98(1994)).

As used herein, the term “nucleic acid molecule” is used interchangeably with “nucleic acid”, “oligonucleotide”, and “polynucleotide”, including cDNA, mRNA, genomic DNA, and the like. As used herein, nucleic acid and nucleic acid molecule may be included by the concept of the term “gene”. A nucleic acid molecule encoding the sequence of a given gene includes “splice mutant (variant)”. Similarly, a particular protein encoded by a nucleic acid encompasses any protein encoded by a splice variant of that nucleic acid. “Splice mutants”, as the name suggests, are products of alternative splicing of a gene. After transcription, an initial nucleic acid transcript may be spliced such that different (alternative) nucleic acid splice products encode different polypeptides. Mechanisms for the production of splice variants vary, but include alternative splicing of exons. Alternative polypeptides derived from the same nucleic acid by read-through transcription are also encompassed by this definition. Any products of a splicing reaction, including recombinant forms of the splice products, are included in this definition. Therefore, for example, Stm1 gene may herein include a spliced mutant of Stm1. The Stm1 gene herein includes an implantation product including the whole or a part of the Stm1 gene region.

As used herein, the term “composite molecule” refers to a molecule in which a plurality of molecules, such as polypeptides, polynucleotides, lipids, sugars, small molecules, or the like, are linked together. Examples of a composite molecule include, but are not limited to, glycolipids, glycopeptides, and the like. Such composite molecules can be herein used as long as they have a similar function to that of the Stm gene or a product thereof.

As used herein, the term “isolated” biological agent (e.g., nucleic acid, protein, or the like) refers to a biological agent that is substantially separated or purified from other biological agents in cells of a naturally-occurring organism (e.g., in the case of nucleic acids, agents other than nucleic acids and a nucleic acid having nucleic acid sequences other than an intended nucleic acid; and in the case of proteins, agents other than proteins and proteins having an amino acid sequence other than an intended protein). The “isolated” nucleic acids and proteins include nucleic acids and proteins purified by a standard purification method. The isolated nucleic acids and proteins also include chemically synthesized nucleic acids and proteins.

As used herein, the term “purified” biological agent (e.g., nucleic acids, proteins, and the like) refers to one from which at least a part of naturally accompanying agents is removed. Therefore, ordinarily, the purity of a purified biological agent is higher than that of the biological agent in a normal state (i.e., concentrated).

As used herein, the terms “purified” and “isolated” mean that the same type of biological agent is present preferably at least 75% by weight, more preferably at least 85% by weight, even more preferably at least 95% by weight, and most preferably at least 98% by weight.

As used herein, the term “gene” refers to an element defining a genetic trait. A gene is typically arranged in a given sequence on a chromosome. A gene which defines the primary structure of a protein is called a structural gene. A gene which regulates the expression of a structural gene is called a regulatory gene (e.g., promoter). Genes herein include structural genes and regulatory genes unless otherwise specified. Therefore, the Stm gene typically includes a structure gene of Stm and a promoter of Stm. As used herein, “gene” may refer to “polynucleotide”, “oligonucleotide”, “nucleic acid”, and “nucleic acid molecule” and/or “protein”, “polypeptide”, “oligopeptide” and “peptide”. As used herein, “gene product” includes “polynucleotide”, “oligonucleotide”, “nucleic acid” and “nucleic acid molecule” and/or “protein”, “polypeptide”, “oligopeptide” and “peptide”, which are expressed by a gene. Those skilled in the art understand what a gene product is, according to the context.

As used herein, the term “homology” in relation to a gene (e.g., a nucleic acid sequence, an amino acid sequence, etc.) refers to the proportion of identity between two or more gene sequences. Therefore, the greater the homology between two given genes, the greater the identity or similarity between their sequences. Whether or not two genes have homology is determined by comparing their sequences directly or by a hybridization method under stringent conditions. When two gene sequences are directly compared with each other, these genes have homology if the DNA sequences of the genes have representatively at least 50% identity, preferably at least 70% identity, more preferably at least 80%, 90%, 95%, 96%, 97%, 98%, or 99% identity with each other. As used herein, the term “similarity” in relation to a gene (e.g., a nucleic acid sequence, an amino acid sequence, or the like) refers to the proportion of identity between two or more sequences when conservative substitution is regarded as positive (identical) in the above-described homology. Therefore, homology and similarity differ from each other in the presence of conservative substitutions. If no conservative substitutions are present, homology and similarity have the same value.

The similarity, identity and homology of amino acid sequences and base sequences are herein compared using BLAST (sequence analyzing tool) with the default parameters.

As used herein, the term “amino acid” may refer to a naturally-occurring or nonnaturally-occurring amino acid as long as the object of the present invention is satisfied. The term “amino acid derivative” or “amino acid analog” refers to an amino acid which is different from a naturally-occurring amino acid and has a function similar to that of the original amino acid. Such amino acid derivatives and amino acid analogs are well known in the art.

The term “naturally-occurring amino acid” refers to an L-isomer of a naturally-occurring amino acid. The naturally-occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine, and lysine. Unless otherwise indicated, all amino acids as used herein are L-isomers. An embodiment using a D-isomer of an amino acid falls within the scope of the present invention.

The term “nonnaturally-occurring amino acid” refers to an amino acid which is ordinarily not found in nature. Examples of nonnaturally-occurring amino acids include D-form of amino acids as described above, norleucine, para-nitrophenylalanine, homophenylalanine, para-fluorophenylalanine, 3-amino-2-benzyl propionic acid, D- or L-homoarginine, and D-phenylalanine. The term “amino acid analog” refers to a molecule having a physical property and/or function similar to that of amino acids, but is not an amino acid. Examples of amino acid analogs include, for example, ethionine, canavanine, 2-methylglutamine, and the like. An amino acid mimic refers to a compound which has a structure different from that of the general chemical structure of amino acids but which functions in a manner similar to that of naturally-occurring amino acids.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “corresponding” amino acid or nucleic acid refers to an amino acid or nucleotide in a given polypeptide or polynucleotide molecule, which has, or is anticipated to have, a function similar to that of a predetermined amino acid or nucleotide in a polypeptide or polynucleotide as a reference for comparison. Particularly, in the case of enzyme molecules, the term refers to an amino acid which is present at a similar position in an active site and similarly contributes to catalytic activity. For example, in the case of antisense molecules for a certain polynucleotide, the term refers to a similar portion in an ortholog corresponding to a particular portion of the antisense molecule. The Stm2 gene and the Stm1 gene herein have a different portion therebetween. Such a different portion can be said to correspond to Stm2 genes and Stm1 genes in other species.

As used herein, the term “corresponding” gene (e.g., a polypeptide or polynucleotide molecule) refers to a gene in a given species, which has, or is anticipated to have, a function similar to that of a predetermined gene in a species as a reference for comparison. When there are a plurality of genes having such a function, the term refers to a gene having the same evolutionary origin. Therefore, a gene corresponding to a given gene may be an ortholog of the given gene. Therefore, genes corresponding to mouse Stm genes can be found in other animals. Such a corresponding gene can be identified by techniques well known in the art. Therefore, for example, a corresponding gene in a given animal can be found by searching a sequence database of the animal (e.g., human, rat) using the sequence of a reference gene (e.g., mouse Stm1 gene, etc.) as a query sequence.

As used herein, the term “nucleotide” may be either naturally-occurring or nonnaturally-occurring. The term “nucleotide derivative” or “nucleotide analog” refers to a nucleotide which is different from naturally-occurring nucleotides and has a function similar to that of the original nucleotide. Such nucleotide derivatives and nucleotide analogs are well known in the art. Examples of such nucleotide derivatives and nucleotide analogs include, but are not limited to, phosphorothioate, phosphoramidate, methylphosphonate, chiral-methylphosphonate, 2-O-methyl ribonucleotide, and peptide-nucleic acid (PNA).

As used herein, the term “fragment” with respect to a polypeptide or polynucleotide refer to a polypeptide or polynucleotide having a sequence length ranging from 1 to n−1 with respect to the full length of the reference polypeptide or polynucleotide (of length n). The length of the fragment can be appropriately changed depending on the purpose. For example, in the case of polypeptides, the lower limit of the length of the fragment includes 3, 4, 5, 6, 7, 8, 9, 10, 15, 20, 25, 30, 40, 50 or more nucleotides. Lengths represented by integers which are not herein specified (e.g., 11 and the like) may be appropriate as a lower limit. For example, in the case of polynucleotides, the lower limit of the length of the fragment includes 5, 6, 7, 8, 9, 10, 15, 20, 25, 30, 40, 50, 75, 100 or more nucleotides. Lengths represented by integers which are not herein specified (e.g., 11 and the like) may be appropriate as a lower limit. As used herein, the length of polypeptides or polynucleotides can be represented by the number of amino acids or nucleic acids, respectively. However, the above-described numbers are not absolute. The above-described numbers as the upper or lower limit are intended to include some greater or smaller numbers (e.g., ±10%), as long as the same function is maintained. For this purpose, “about” may be herein put ahead of the numbers. However, it should be understood that the interpretation of numbers is not affected by the presence or absence of “about” in the present specification.

As used herein, the term “Stm” or “Stm gene” refers to all genes having any homology to a DNA base sequence of the Stm1 gene which is observed in comparison. Some genes whose expression is observed are expressed in either an undifferentiated cell or an early embryo or germ cell, or in some cells. Such a Stm gene includes, but is not limited to, for example,

(A) a nucleic acid molecule, comprising:

(a) a polynucleotide having a base sequence set forth in SEQ ID NO. 1, 3, 5, 7, 9 or 29, or a fragment thereof;

(b) a polynucleotide encoding a polypeptide consisting of an amino acid sequence set forth in SEQ ID NO. 2, 4, 6, 8, 10 or 30, or a fragment thereof;

(c) a polynucleotide encoding a variant polypeptide having an amino acid sequence set forth in SEQ ID NO. 2, 4, 6, 8, 10 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5, 7, 9 or 29, or a fragment thereof;

(e) a polynucleotide encoding a species homolog of a polypeptide consisting of an amino acid sequence set forth in SEQ ID NO. 2, 4, 6, 8, 10 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity, or

(B) a nucleic acid molecule encoding a polypeptide including:

(a) a polypeptide consisting of an amino acid sequence set forth in SEQ ID NO. 2, 4, 6, 8, 10 or 30, or a fragment thereof;

(b) a polypeptide having an amino acid sequence set forth in SEQ ID NO. 2, 4, 6, 8, 10 or 30, or a fragment thereof, wherein at least one amino acid in the sequence has a mutation selected from the group consisting of substitution, addition, and deletion, and wherein the variant polypeptide has biological activity;

(c) a polypeptide encoded by a spliced mutant or alleic mutant of a base sequence set forth in SEQ ID NO. 1, 3, 5, 7, 9 or 29;

(d) a polypeptide being a species homolog of an amino acid sequence set forth in SEQ ID NO. 2, 4, 6, 8, 10 or 30;

(e) a polypeptide having at least 70% identity to any one of the polypeptides of (a) to (d) and having biological activity.

Preferably, a Stm gene includes, but is not limited to,

(A) a nucleic acid molecule, comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity, or

(B) a nucleic acid molecule encoding a polypeptide including:

(a) a polypeptide consisting of an amino acid sequence set forth in SEQ ID NO. 2, 4, 6 or 30, or a fragment thereof;

(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;

(c) a polypeptide encoded by a spliced mutant or alleic mutant of a base sequence set forth in SEQ ID NO. 1, 3, 5 or 29;

(d) a polypeptide being a species homolog of an amino acid sequence set forth in SEQ ID NO. 2, 4, 6 or 30;

(e) a polypeptide having at least 70% identity to anyone of the polypeptides of (a) to (d) and having biological activity.

A Stm gene includes a Stm1 gene, a Stm2 gene, a Stm3 gene, and a Stm4 gene. If particularly specified herein, a Stm gene may be described in italic type, a Stm gene of mouse is designated as Stm, and a Stm gene of human may be designated as STM, however, they usually do not mean a specific type. A protein as a product of a Stm gene may be designated as non-slanting STM, which usually does not mean a specific type.

As used herein, the terms “Stm1” and “Stm1 gene” refer to a nucleic acid sequence set forth in SEQ ID NO. 1, 3, 5 or 29 or a gene comprising a nucleic acid sequence encoding an amino acid sequence set forth in SEQ ID NO. 2, 4, 6 or 30, and a corresponding gene thereto (including a species homolog). To specify a gene product of Stm1, preferably, an antibody specific to a polypeptide comprising the full length amino acid sequence is used. It is known that the Stm1 gene is the same as a gene called Nanog.

As used herein, the terms “Stm2” and “Stm2 gene” refer to a gene comprising a nucleic acid sequence set forth in SEQ ID NO. 7 or 9 or a nucleic acid sequence encoding an amino acid sequence set forth in SEQ ID NO. 8 or 10, or a corresponding gene thereto (including species homologs). Mouse Stm2 is a gene having 99.6% homology to Stm1 with respect to the base sequence of a region encoding mRNA. However, the Stm2 gene has a gene structure consisting of a single exon without any intron, and is thus different in structure from Stm1 having 4 exons and 3 introns. It is also known that Stm1 and Stm2 are located in different chromosomes. According to the present invention, it was revealed that Stm2 is positioned on mouse 7th chromosome 7E3. Note that Stm1 and Stm2 have 99.6% homology in mouse.

A crucial difference between Stm1 and Stm2 is the presence or absence of expression in a cell. Whereas Stm1 is a gene which is expressed, Stm2 is not expressed in typical cells. Thus, Stm2 has been revealed to be a pseudogene.

As used herein, the terms “STM”, “STM protein”, “STM1”, “STM1 protein”, “STM2”, and “STM2 protein” are used to indicate the protein form of a corresponding gene (Stm, Stm1, Stm2, etc.).

As used herein, the term “promoter sequence of Stm1” refers to a promoter sequence associated with a Stm1 gene. Examples of such a sequence include, but are not limited to, a sequence set forth in SEQ ID NO. 34 (mouse) and a corresponding sequence, and the like. For the control of expression of a Stm1 gene, a promoter is preferably located at 390 bp upstream of a transcription start site. Examples of the base sequence of such a promoter include, but are not limited to, sequences set forth in SEQ ID NO. 31 (human), 32 (mouse), 33 (cynomolgus monkey), and the like. Among these sequences, Oct/Sox (positions −180 to −166 where a transcription start site is a starting point, TTTTGCAT TACAATG (Oct/Sox motif sequence; where TTTTGCAT is a Oct motif sequence, and TACAATG is a Sox motif sequence)) is a motif.

As used herein, the term “exogenous gene” in relation to a certain organism refers to a gene which is not naturally present in the organism. Such an exogenous gene may be a gene which is naturally present in the organism but is modified, a gene which is naturally present in other organisms (e.g., a Stm1 gene, etc.), an artificially synthesized gene, a composite thereof (e.g., a fusion, etc.). An organism containing such an exogenous gene may express a nonnaturally-occurring gene product.

The term “cytokine” is used herein in the broadest sense in the art and refers to a physiologically active substance which is produced from a cell and acts on the same or different cell. Cytokines are generally proteins or polypeptides having a function of controlling an immune response, regulating the endocrine system, regulating the nervous system, acting against a tumor, acting against a virus, regulating cell growth, regulating cell differentiation, or the like. Cytokines are herein in the form of a protein or a nucleic acid or in other forms. In actual practice, cytokines are typically proteins. The terms “growth factor” refers to a substance which promotes or controls cell growth. Growth factors are also called “proliferation factor” or “development factor”. Growth factors may be added to cell or tissue culture medium, substituting for serum macromolecules. It has been revealed that a number of growth factors have a function of controlling differentiation in addition to a function of promoting cell growth. Examples of cytokines representatively include, but are not limited to, interleukins, chemokines, hematopoietic factors such as colony stimulating factors, a tumor necrosis factor, interferons. Representative examples of growth factors include, but are not limited to, a platelet-derived growth factor (PDGF), an epidermal growth factor (EGF), a fibroblast growth factor (FGF), a hepatocyte growth factor (HGF), an endothelial cell growth factor (VEGF), cardiotrophin, and the like, which have proliferative activity.

In the present invention, an exogenous gene to be expressed may be used, which has homology to the above-described naturally-occurring exogenous gene. Examples of such an exogenous gene having homology include, but are not limited to, when Blast is used using default parameters, a nucleic acid molecule having a nucleic acid sequence having at least about 30%, about 35%, about 40%, about 45%, about 50%, about 55%, about 60%, about 65%, about 70%, about 75%, about 80%, about 85%, about 90%, about 95%, or about 99% identity or similarity to a reference exogenous gene for comparison, or a polypeptide molecule having an amino acid sequence having at least about 30%, about 35%, about 40%, about 45%, about 50%, about 55%, about 60%, about 65%, about 70%, about 75%, about 80%, about 85%, about 90%, about 95%, or about 99% identity or similarity to a reference exogenous gene for comparison.

As used herein, the term “expression” of a gene, a polynucleotide, a polypeptide, or the like, indicates that the gene or the like is affected by a predetermined action in vivo to be changed into another form. Preferably, the term “expression” indicates that genes, polynucleotides, or the like are transcribed and translated into polypeptides. In one embodiment of the present invention, genes may be transcribed into mRNA. More preferably, these polypeptides may have post-translational processing modifications.

Therefore, as used herein, the term “reduction” of “expression” of a gene, a polynucleotide, a polypeptide, or the like indicates that the level of expression is significantly reduced in the presence of the action of the agent of the present invention as compared to when the action of the agent is absent. Preferably, the reduction of expression includes a reduction in the amount of expression of a polypeptide. As used herein, the term “increase” of “expression” of a gene, a polynucleotide, a polypeptide, or the like indicates that the level of expression is significantly increased in the presence of the action of the agent of the present invention as compared to when the action of the agent is absent. Preferably, the increase of expression includes an increase in the amount of expression of a polypeptide. As used herein, the term “induction” of expression of a gene indicates that the amount of expression of the gene is increased by applying a given agent to a given cell. Therefore, the induction of expression includes allowing a gene to be expressed when expression of the gene is not otherwise observed, and increasing the amount of expression of the gene when expression of the gene is observed.

As used herein, the term “biological activity” refers to activity possessed by an agent (e.g., a polynucleotide, a protein, etc.) within an organism, including activities exhibiting various functions (e.g., transcription promoting activity, etc.). For example, when a certain factor is an enzyme, the biological activity thereof includes its enzyme activity. In another example, when a certain factor is a ligand, the biological activity thereof includes the binding of the ligand to a receptor corresponding thereto. The above-described biological activity can be measured by techniques well-known in the art.

As used herein, the term “antisense (activity)” refers to activity which permits specific suppression or reduction of expression of a target gene. The antisense activity is ordinarily achieved by a nucleic acid sequence having a length of at least 8 contiguous nucleotides, which is complementary to the nucleic acid sequence of a target gene (e.g., Stm, etc.). A molecule having such antisense activity is called an antisense molecule. Such a nucleic acid sequence preferably has a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 30 contiguous nucleotides, a length of at least 40 contiguous nucleotides, and a length of at least 50 contiguous nucleotides. These nucleic acid sequences include nucleic acid sequences having at least 70% homology thereto, more preferably at least 80%, even more preferably at least 90%, and still even more preferably at least 95%. The antisense activity is preferably complementary to a 5′ terminal sequence of the nucleic acid sequence of a target gene. Such an antisense nucleic acid sequence includes the above-described sequences having one or several, or at least one, nucleotide substitutions, additions, and/or deletions.

As used herein, the term “RNAi” is an abbreviation of RNA interference and refers to a phenomenon that an agent for causing RNAi, such as double-stranded RNA (also called dsRNA), is introduced into cells and mRNA homologous thereto is specifically degraded, so that synthesis of gene products is suppressed, and a technique using the phenomenon. As used herein, RNAi may have the same meaning as that of an agent which causes RNAi.

As used herein, the term “an agent causing RNAi” refers to any agent capable of causing RNAi. As used herein, “an agent causing RNAi for a gene” indicates that the agent causes RNAi relating to the gene and the effect of RNAi is achieved (e.g., suppression of expression of the gene, and the like). Examples of such an agent causing RNAi include, but are not limited to, a sequence having at least about 70% homology to the nucleic acid sequence of a target gene or a sequence hybridizable under stringent conditions, RNA containing a double-stranded portion having a length of at least 10 nucleotides or variants thereof. Here, this agent may be preferably DNA containing a 3′ protruding end, and more preferably the 3′ protruding end has a length of 2 or more nucleotides (e.g., 2-4 nucleotides in length).

Though not wishing to be bound by any theory, a mechanism which causes RNAi is considered as follows. When a molecule which causes RNAi, such as dsRNA, is introduced into a cell, an RNaseIII-like nuclease having a helicase domain (called dicer) cleaves the molecule on about a 20 base pair basis from the 3′ terminus in the presence of ATP in the case where the RNA is relatively long (e.g., 40 or more base pairs). As used herein, the term “siRNA” is an abbreviation of short interfering RNA and refers to short double-stranded RNA of 10 or more base pairs which are artificially chemically or biochemically synthesized, synthesized in the organism body, or produced by double-stranded RNA of about 40 or more base pairs being degraded within the body. siRNA typically has a structure having 5′-phosphate and 3′-OH, where the 3′ terminus projects by about 2 bases. A specific protein is bound to siRNA to form RISC(RNA-induced-silencing-complex). This complex recognizes and binds to mRNA having the same sequence as that of siRNA and cleave mRNA at the middle of siRNA due to RNaseIII-like enzymatic activity. It is preferable that the relationship between the sequence of siRNA and the sequence of mRNA to be cleaved as a target is a 100% match. However, base mutation at a site away from the middle of siRNA does not completely remove the cleavage activity by RNAi, leaving partial activity, while base mutation in the middle of siRNA has a large influence and the mRNA cleavage activity by RNAi is considerably lowered. By utilizing such a nature, only mRNA having a mutation can be specifically degraded. Specifically, siRNA in which the mutation is provided in the middle thereof is synthesized and is introduced into a cell. Therefore, in the present invention, siRNA per se as well as an agent capable of producing siRNA (e.g., representatively dsRNA of about 40 or more base pairs) can be used as an agent capable of eliciting RNAi.

Also, though not wishing to be bound by any theory, apart from the above-described pathway, the antisense strand of siRNA binds to mRNA and siRNA functions as a primer for RNA-dependent RNA polymerase (RdRP), so that dsRNA is synthesized. This dsRNA is a substrate for a dicer again, leading to production of new siRNA. It is intended that such an action is amplified. Therefore, in the present invention, siRNA per se as well as an agent capable of producing siRNA are useful. In fact, in insects and the like, for example, 35 dsRNA molecules can substantially completely degrade 1,000 or more copies of intracellular mRNA, and therefore, it will be understood that siRNA per se as well as an agent capable of producing siRNA are useful.

In the present invention, double-stranded RNA having a length of about 20 bases (e.g., representatively about 21 to 23 bases) or less than about 20 bases, which is called siRNA, can be used. Expression of siRNA in cells can suppress expression of a pathogenic gene targeted by the siRNA. Therefore, siRNA can be used for treatment, prophylaxis, prognosis, and the like of diseases.

The siRNA of the present invention may be in any form as long as it can elicit RNAi.

In another embodiment, an agent capable of causing RNAi may have a short hairpin structure having a sticky portion at the 3′ terminus (shRNA; short hairpin RNA). As used herein, the term “shRNA” refers to a molecule of about 20 or more base pairs in which a single-stranded RNA partially contains a palindromic base sequence and forms a double-strand structure therein (i.e., a hairpin structure). shRNA can be artificially chemically synthesized. Alternatively, shRNA can be produced by linking sense and antisense strands of a DNA sequence in reverse directions and synthesizing RNA in vitro with T7 RNA polymerase using the DNA as a template. Though not wishing to be bound by any theory, it should be understood that after shRNA is introduced into a cell, the shRNA is degraded in the cell into a length of about 20 bases (e.g., representatively 21, 22, 23 bases), and causes RNAi as with siRNA, leading to the treatment effect of the present invention. It should be understood that such an effect is exhibited in a wide range of organisms, such as insects, plants, animals (including mammals), and the like. Thus, shRNA elicits RNAi as with siRNA and therefore can be used as an effective component of the present invention. shRNA may preferably have a 3′ protruding end. The length of the double-stranded portion is not particularly limited, but is preferably about 10 or more nucleotides, and more preferably about 20 or more nucleotides. Here, the 3′ protruding end may be preferably DNA, more preferably DNA of at least 2 nucleotides in length, and even more preferably DNA of 2-4 nucleotides in length.

An agent capable of causing RNAi used in the present invention may be artificially synthesized (chemically or biochemically) or naturally occurring. There is substantially no difference therebetween in terms of the effect of the present invention. A chemically synthesized agent is preferably purified by liquid chromatography or the like.

An agent capable of causing RNAi used in the present invention can be produced in vitro. In this synthesis system, T7 RNA polymerase and T7 promoter are used to synthesize antisense and sense RNAs from template DNA. These RNAs are annealed and thereafter are introduced into a cell. In this case, RNAi is caused via the above-described mechanism, thereby achieving the effect of the present invention. Here, for example, the introduction of RNA into cell can be carried out by a calcium phosphate method.

Another example of an agent capable of causing RNAi according to the present invention is a single-stranded nucleic acid hybridizable to mRNA or all nucleic acid analogs thereof. Such agents are useful for the method and composition of the present invention.

As used herein, “polynucleotides hybridizing under stringent conditions” refers to conditions commonly used and well known in the art. Such a polynucleotide can be obtained by conducting colony hybridization, plaque hybridization, Southern blot hybridization, or the like using a polynucleotide selected from the polynucleotides of the present invention. Specifically, a filter on which DNA derived from a colony or plaque is immobilized is used to conduct hybridization at 65° C. in the presence of 0.7 to 1.0 M NaCl. Thereafter, a 0.1 to 2-fold concentration SSC (saline-sodium citrate) solution (1-fold concentration SSC solution is composed of 150 mM sodium chloride and 15 mM sodium citrate) is used to wash the filter at 65° C. Polynucleotides identified by this method are referred to as “polynucleotides hybridizing under stringent conditions”. Hybridization can be conducted in accordance with a method described in, for example, Molecular Cloning 2nd ed., Current Protocols in Molecular Biology, Supplement 1-38, DNA Cloning 1: Core Techniques, A Practical Approach, Second Edition, Oxford University Press (1995), and the like. Here, sequences hybridizing under stringent conditions exclude, preferably, sequences containing only A or T. “Hybridizable polynucleotide” refers to a polynucleotide which can hybridize other polynucleotides under the above-described hybridization conditions. Specifically, the hybridizable polynucleotide includes at least a polynucleotide having a homology of at least 60% to the base sequence of DNA encoding a polypeptide having an amino acid sequence specifically herein disclosed, preferably a polynucleotide having a homology of at least 80%, and more preferably a polynucleotide having a homology of at least 95%.

As used herein, the term “probe” refers to a substance for use in searching, which is used in a biological experiment, such as in vitro and/or in vivo screening or the like, including, but not being limited to, for example, a nucleic acid molecule having a specific base sequence or a peptide containing a specific amino acid sequence.

Examples of a nucleic acid molecule as a common probe include one having a nucleic acid sequence having a length of at least 8 contiguous nucleotides, which is homologous or complementary to the nucleic acid sequence of a gene of interest. Such a nucleic acid sequence may be preferably a nucleic acid sequence having a length of at least 9 contiguous nucleotides, more preferably a length of at least 10 contiguous nucleotides, and even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 20 contiguous nucleotides, a length of at least 25 contiguous nucleotides, a length of at least 30 contiguous nucleotides, a length of at least 40 contiguous nucleotides, or a length of at least 50 contiguous nucleotides. A nucleic acid sequence used as a probe includes a nucleic acid sequence having at least 70% homology to the above-described sequence, more preferably at least 80%, and even more preferably at least 90% or at least 95%.

As used herein, the term “search” indicates that a given nucleic acid sequence is utilized to find other nucleic acid base sequences having a specific function and/or property either electronically or biologically, or using other methods. Examples of an electronic search include, but are not limited to, BLAST (Altschul et al., J. Mol. Biol. 215:403-410 (1990)), FASTA (Pearson & Lipman, Proc. Natl. Acad. Sci., USA 85:2444-2448 (1988)), Smith and Waterman method (Smith and Waterman, J. Mol. Biol. 147:195-197 (1981)), and Needleman and Wunsch method (Needleman and Wunsch, J. Mol. Biol. 48:443-453 (1970)), and the like. Examples of a biological search include, but are not limited to, a macroarray in which genomic DNA is attached to a nylon membrane or the like or a microarray (microassay) in which genomic DNA is attached to a glass plate under stringent hybridization, PCR and in situ hybridization, and the like.

As used herein, the term “primers refers to a substance required for initiation of a reaction of a macromolecule compound to be synthesized, in a macromolecule synthesis enzymatic reaction. In a reaction for synthesizing a nucleic acid molecule, a nucleic acid molecule (e.g., DNA, RNA, or the like) which is complementary to part of a macromolecule compound to be synthesized may be used.

A nucleic acid molecule which is ordinarily used as a primer includes one that has a nucleic acid sequence having a length of at least 8 contiguous nucleotides, which is complementary to the nucleic acid sequence of a gene of interest. Such a nucleic acid sequence preferably has a length of at least 9 contiguous nucleotides, more preferably a length of at least 10 contiguous nucleotides, even more preferably a length of at least 11 contiguous nucleotides, a length of at least 12 contiguous nucleotides, a length of at least 13 contiguous nucleotides, a length of at least 14 contiguous nucleotides, a length of at least 15 contiguous nucleotides, a length of at least 16 contiguous nucleotides, a length of at least 17 contiguous nucleotides, a length of at least 18 contiguous nucleotides, a length of at least 19 contiguous nucleotides, a length of at least 20 contiguous nucleotides, a length of at least 25 contiguous nucleotides, a length of at least 30 contiguous nucleotides, a length of at least 40 contiguous nucleotides, and a length of at least 50 contiguous nucleotides. A nucleic acid sequence used as a primer includes a nucleic acid sequence having at least 70% homology to the above-described sequence, more preferably at least 80%, even more preferably at least 90%, and most preferably at least 95%. An appropriate sequence as a primer may vary depending on the property of the sequence to be synthesized (amplified). Those skilled in the art can design an appropriate primer depending on the sequence of interest. Such primer design is well known in the art and may be performed manually or using a computer program (e.g., LASERGENE, Primer Select, DNAStar).

As used herein, the term “epitope” refers to an antigenic determinant. Therefore, the term “epitope” includes a set of amino acid residues which is involved in recognition by a particular immunoglobulin, or in the context of T cells, those residues necessary for recognition by T cell receptor proteins and/or Major Histocompatibility Complex (MHC) receptors. This term is also used interchangeably with “antigenic determinant” or “antigenic determinant site”. In the field of immunology, in vivo or in vitro, an epitope is the features of a molecule (e.g., primary, secondary and tertiary peptide structure, and charge) that form a site recognized by an immunoglobulin, T cell receptor or HLA molecule. An epitope including a peptide comprises 3 or more amino acids in a spatial conformation which is unique to the epitope. Generally, an epitope consists of at least 5 such amino acids, and more ordinarily, consists of at least 6, 7, 8, 9 or 10 such amino acids. The greater the length of an epitope, the more the similarity of the epitope to the original peptide, i.e., longer epitopes are generally preferable. This is not necessarily the case when the conformation is taken into account. Methods of determining the spatial conformation of amino acids are known in the art, and include, for example, X-ray crystallography and 2-dimensional nuclear magnetic resonance spectroscopy. Furthermore, the identification of epitopes in a given protein is readily accomplished using techniques well known in the art. See, also, Geysen et al., Proc. Natl. Acad. Sci. USA (1984) 81: 3998 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular immunology (1986) 23: 709 (technique for identifying peptides with high affinity for a given antibody). Antibodies that recognize the same epitope can be identified in a simple immunoassay. Thus, methods for determining an epitopes including a peptide are well known in the art. Such an epitope can be determined using a well-known, common technique by those skilled in the art if the primary nucleic acid or amino acid sequence of the epitope is provided.

Therefore, an epitope including a peptide requires a sequence having a length of at least 3 amino acids, preferably at least 4 amino acids, more preferably at least 5 amino acids, at least 6 amino acids, at least 7 amino acids, at least 8 amino acids, at least 9 amino acids, at least 10 amino acids, at least 15 amino acids, at least 20 amino acids, and 25 amino acids. Epitopes may be linear or conformational.

As used herein, the term “agent capable of binding specifically to” a certain nucleic acid molecule or polypeptide refers to an agent which has a level of binding to the nucleic acid molecule or polypeptide equal to or higher than a level of binding to other nucleic acid molecules or polypeptides. Examples of such an agent include, but are not limited to, when a target is a nucleic acid molecule, a nucleic acid molecule having a complementary sequence of a nucleic acid molecule of interest, a polypeptide capable of binding to a nucleic acid sequence of interest (e.g., a transcription agent; etc.), and the like, and when a target is a polypeptide, an antibody, a single chain antibody, either of a pair of a receptor and a ligand, either of a pair of an enzyme and a substrate, and the like.

As used herein, the term “antibody” encompasses polyclonal antibodies, monoclonal antibodies, human antibodies, humanized antibodies, polyfunctional antibodies, chimeric antibodies, and anti-idiotype antibodies, and fragments thereof (e.g., F(ab′)2 and Fab fragments), and other recombinant conjugates. These antibodies may be fused with an enzyme (e.g., alkaline phosphatase, horseradish peroxidase, α-galactosidase, and the like) via a covalent bond or by recombination.

As used herein, the term “monoclonal antibody” refers to an antibody composition having a group of homologous antibodies. This term is not limited by the production manner thereof. This term encompasses all immunoglobulin molecules and Fab molecules, F(ab′)2 fragments, Fv fragments, and other molecules having an immunological binding property of the original monoclonal antibody molecule. Methods for producing polyclonal antibodies and monoclonal antibodies are well known in the art, and will be more sufficiently described below.

Monoclonal antibodies are prepared by using the standard technique well known in the art (e.g., Kohler and Milstein, Nature (1975) 256:495) or a modification thereof (e.g., Buck et al. (1982) in Vitro 18:377). Representatively, a mouse or rat is immunized with a protein bound to a protein carrier, and boosted. Subsequently, the spleen (and optionally several large lymph nodes) is removed and dissociated into single cells. If desired, the spleen cells may be screened (after removal of nonspecifically adherent cells) by applying a cell suspension to a plate or well coated with a protein antigen. B-cells that express membrane-bound immunoglobulin specific for the antigen bind to the plate, and are not rinsed away with the rest of the suspension. Resulting B-cells, or all dissociated spleen cells, are then induced to fuse with myeloma cells to form hybridomas. The hybridomas are used to produce monoclonal antibodies.

As used herein, the term “antigen” refers to any substrate to which an antibody molecule may specifically bind. As used herein, the term “immunogen” refers to an antigen capable of initiating activation of the antigen-specific immune response of a lymphocyte.

In a given protein molecule, a given amino acid contained in a sequence may be substituted with another amino acid in a protein structure, such as a cationic region or a substrate molecule binding site, without a clear reduction or loss of interactive binding ability. A given biological function of a protein is defined by the interactive ability or other property of the protein. Therefore, a particular amino acid substitution may be performed in an amino acid sequence, or at the DNA code sequence level, to produce a protein which maintains the original property after the substitution. Therefore, various modifications of peptides as disclosed herein and DNA encoding such peptides may be performed without clear losses of biological usefulness.

(Modification of Genes)

When the above-described modifications are designed, the hydrophobicity indices of amino acids may be taken into consideration. The hydrophobic amino acid indices play an important role in providing a protein with an interactive biological function, which is generally recognized in the art (Kyte, J. and Doolittle, R. F., J. Mol. Biol. 157(1):105-132, 1982). The hydrophobic property of an amino acid contributes to the secondary structure of a protein and then regulates interactions between the protein and other molecules (e.g., enzymes, substrates, receptors, DNA, antibodies, antigens, etc.). Each amino acid is given a hydrophobicity index based on the hydrophobicity and charge properties thereof as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5).

It is well known that if a given amino acid is substituted with another amino acid having a similar hydrophobicity index, the resultant protein may still have a biological function similar to that of the original protein (e.g., a protein having an equivalent enzymatic activity). For such an amino acid substitution, the hydrophobicity index is preferably within ±2, more preferably within ±1, and even more preferably within ˜0.5. It is understood in the art that such an amino acid substitution based on hydrophobicity is efficient.

A hydrophilicity index is also useful for modification of an amino acid sequence of the present invention. As described in U.S. Pat. No. 4,554,101, amino acid residues are given the following hydrophilicity indices: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid may be substituted with another amino acid which has a similar hydrophilicity index and can still provide a biological equivalent. For such an amino acid substitution, the hydrophilicity index is preferably within ±2, more preferably ±1, and even more preferably ±0.5.

The term “conservative substitution” as used herein refers to amino acid substitution in which a substituted amino acid and a substituting amino acid have similar hydrophilicity indices or/and hydrophobicity indices. For example, the conservative substitution is carried out between amino acids having a hydrophilicity or hydrophobicity index of within ±2, preferably within ±1, and more preferably within ±0.5. Examples of the conservative substitution include, but are not limited to, substitutions within each of the following residue pairs: arginine and lysine; glutamic acid and aspartic acid; serine and threonine; glutamine and asparagine; and valine, leucine, and isoleucine, which are well known to those skilled in the art.

As used herein, the term “variant” refers to a substance, such as a polypeptide, polynucleotide, or the like, which differs partially from the original substance. Examples of such a variant include a substitution variant, an addition variant, a deletion variant, a truncated variant, an allelic variant, and the like. Examples of such a variant include, but are not limited to, a nucleotide or polypeptide having one or several substitutions, additions and/or deletions or a nucleotide or polypeptide having at least one substitution, addition and/or deletion. The term “allele” as used herein refers to a genetic variant located at a locus identical to a corresponding gene, where the two genes are distinguished from each other. Therefore, the term “allelic variant” as used herein refers to a variant which has an allelic relationship with a given gene. Such an allelic variant ordinarily has a sequence the same as or highly similar to that of the corresponding allele, and ordinarily has almost the same biological activity, though it rarely has different biological activity. The term species homolog” or “homolog” as used herein refers to one that has an amino acid or nucleotide homology with a given gene in a given species (preferably at least 60% homology, more preferably at least 80%, at least 85%, at least 90%, and at least 95% homology). A method for obtaining such a species homolog is clearly understood from the description of the present specification. The term “orthologs” (also called orthologous genes) refers to genes in different species derived from a common ancestry (due to speciation). For example, in the case of the hemoglobin gene family having multigene structure, human and mouse α-hemoglobin genes are orthologs, while the human α-hemoglobin gene and the human β-hemoglobin gene are paralogs (genes arising from gene duplication). Orthologs are useful for estimation of molecular phylogenetic trees. Usually, orthologs in different species may have a function similar to that of the original species. Therefore, orthologs of the present invention may be useful in the present invention.

As used herein, the term “conservative (or conservatively modified) variant” applies to both amino acid and nucleic acid sequences. With respect to particular nucleic acid sequences, conservatively modified variants refer to those nucleic acids which encode identical or essentially identical amino acid sequences. Because of the degeneracy of the genetic code, a large number of functionally identical nucleic acids encode any given protein. For example, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” which represent one species of conservatively modified variation. Every nucleic acid sequence herein which encodes a polypeptide also describes every possible silent variation of the nucleic acid. Those skilled in the art will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence. Preferably, such modification may be performed while avoiding substitution of cysteine which is an amino acid capable of largely affecting the higher-order structure of a polypeptide. Examples of a method for such modification of a base sequence include cleavage using a restriction enzyme or the like; ligation or the like by treatment using DNA polymerase, Klenow fragments, DNA ligase, or the like; and a site specific base substitution method using synthesized oligonucleotides (specific-site directed mutagenesis; Mark Zoller and Michael Smith, Methods in Enzymology, 100, 468-500(1983)). Modification can be performed using methods ordinarily used in the field of molecular biology. Preferably, herein, such a conservative substitution may be advantageously a substitution between portions common to a Stm1 gene and a Stm2 gene. This is because even if such a conservative substitution is performed, a Stm1 gene and a Stm2 gene can be distinguished.

In order to prepare functionally equivalent polypeptides, amino acid additions, deletions, or modifications can be performed in addition to amino acid substitutions. Amino acid substitution(s) refers to the replacement of at least one amino acid of an original peptide with different amino acids, such as the replacement of 1 to 10 amino acids, preferably 1 to 5 amino acids, and more preferably 1 to 3 amino acids with different amino acids. Amino acid addition(s) refers to the addition of at least one amino acid to an original peptide chain, such as the addition of 1 to 10 amino acids, preferably 1 to 5 amino acids, and more preferably 1 to 3 amino acids to an original peptide chain. Amino acid deletion(s) refers to the deletion of at least one amino acid, such as the deletion of 1 to 10 amino acids, preferably 1 to 5 amino acids, and more preferably 1 to 3 amino acids. Amino acid modification includes, but is not limited to, amidation, carboxylation, sulfation, halogenation, truncation, lipidation, alkylation, glycosylation, phosphorylation, hydroxylation, acylation (e.g., acetylation), and the like. Amino acids to be substituted or added may be naturally-occurring or nonnaturally-occurring amino acids, or amino acid analogs. Naturally-occurring amino acids are preferable.

As used herein, the term “peptide analogs or “peptide derivative” refers to a compound which is different from a peptide but has at least one chemical or biological function equivalent to the peptide. Therefore, a peptide analog includes one that has at least one amino acid analog or amino acid derivative addition or substitution with respect to the original peptide. A peptide analog has the above-described addition or substitution so that the function thereof is substantially the same as the function of the original peptide (e.g., a similar pKa value, a similar functional group, a similar binding manner to other molecules, a similar water-solubility, and the like). Such a peptide analog can be prepared using techniques well known in the art. Therefore, a peptide analog may be a polymer containing an amino acid analog.

Similarly, the term “polynucleotide analog” or “nucleic acid analog” refers to a compound which is different from a polynucleotide or a nucleic acid but has at least one chemical function or biological function equivalent to that of a polynucleotide or a nucleic acid. Therefore, a polynucleotide analog or a nucleic acid analog includes one that has at least one nucleotide analog or nucleotide derivative addition or substitution with respect to the original peptide.

Nucleic acid molecules as used herein includes one in which a part of the sequence of the nucleic acid is deleted or is substituted with other base(s), or an additional nucleic acid sequence is inserted, as long as a polypeptide expressed by the nucleic acid has substantially the same activity as that of the naturally-occurring polypeptide, as described above. Alternatively, an additional nucleic acid may be linked to the 5′ terminus and/or 3′ terminus of the nucleic acid. The nucleic acid molecule may include one that is hybridizable to a gene encoding a polypeptide under stringent conditions and encodes a polypeptide having substantially the same function as that of that polypeptide. Such a gene is known in the art and can be used in the present invention.

The above-described nucleic acid can be obtained by a well-known PCR method, i.e., chemical synthesis. This method may be combined with, for example, site-specific mutagenesis, hybridization, or the like.

As used herein, the term “substitution, addition or deletion” for a polypeptide or a polynucleotide refers to the substitution, addition or deletion of an amino acid or its substitute, or a nucleotide or its substitute with respect to the original polypeptide or polynucleotide. This is achieved by techniques well known in the art, including a site-specific mutagenesis technique and the like. A polypeptide or a polynucleotide may have any number (>0) of substitutions, additions, or deletions. The number can be as large as a variant having such a number of substitutions, additions or deletions maintains an intended function (e.g., the information transfer function of hormones and cytokines, etc.). For example, such a number may be one or several, and preferably within 20% or 10% of the full length, or no more than 100, no more than 50, no more than 25, or the like.

As used herein, the term “specifically expressed” in relation to a gene indicates that the gene is expressed in a specific site or for a specific period of time at a level different from (preferably higher than) that in other sites or periods of time. The term “specifically expressed” indicates that a gene may be expressed only in a given site (specific site) or may be expressed in other sites. Preferably, the term “specifically expressed” indicates that a gene is expressed only in a given site.

Molecular biological techniques, biochemical techniques, and microorganism techniques as used herein are well known in the art and commonly used, and are described in, for example, Sambrook J. et al. (1989), Molecular Cloning: A Laboratory Manual, Cold Spring Harbor and its 3rd Ed. (2001); Ausubel, F. M. (1987), Current Protocols in Molecular Biology, Greene Pub. Associates and Wiley-interscience; Ausubel, F. M. (1989), Short Protocols in Molecular Biology: A Compendium of Methods from Current Protocols in Molecular Biology, Greene Pub. Associates and Wiley-interscience; Innis, M. A. (1990), PCR Protocols: A Guide to Methods and Applications, Academic Press; Ausubel, F. M. (1992), Short Protocols in Molecular Biology: A Compendium of Methods from Current Protocols in Molecular Biology, Greene Pub. Associates; Ausubel, F. M. (1995), Short Protocols in Molecular Biology: A Compendium of Methods from Current Protocols in Molecular Biology, Greene Pub. Associates; Innis, M. A. et al. (1995), PCR Strategies, Academic Press; Ausubel, F. M. (1999), Short Protocols in Molecular Biology: A Compendium of Methods from Current Protocols in Molecular Biology, Wiley, and annual updates; Sninsky, J. J. et al. (1999), PCR Applications: Protocols for Functional Genomics, Academic Press; Special issue, Jikken Igaku [Experimental Medicine] “Idenshi Donyu & Hatsugenkaiseki Jikkenho [Experimental Method for Gene introduction & Expression Analysis]”, Yodo-sha, 1997; and the like. Relevant portions (or possibly the entirety) of each of these publications are herein incorporated by reference.

DNA synthesis techniques and nucleic acid chemistry for preparing artificially synthesized genes are described in, for example, Gait, M. J. (1985), Oligonucleotide Synthesis: A Practical Approach, IRL Press; Gait, M. J. (1990), Oligonucleotide Synthesis: A Practical Approach, IRL Press; Eckstein, F. (1991), Oligonucleotides and Analogues: A Practical Approach, IRL Press; Adams, R. L. et al. (1992), The Biochemistry of the Nucleic Acids, Chapman & Hall; Shabarova, Z. et al. (1994), Advanced Organic Chemistry of Nucleic Acids, Weinheim; Blackburn, G. M. et al. (1996), Nucleic Acids in Chemistry and Biology, Oxford University Press; Hermanson, G. T. (1996), Bioconjugate Techniques, Academic Press; and the like, related portions of which are herein incorporated by reference.

When a gene is mentioned herein, the term “vector” or “recombinant vector” refers to a vector capable of transferring a polynucleotide sequence of interest to a target cell. Such a vector is capable of self-replication or incorporation into a chromosome in a host cell (e.g., a prokaryotic cell, yeast, an animal cell, a plant cell, an insect cell, an individual animal, and an individual plant, etc.), and contains a promoter at a site suitable for transcription of a polynucleotide of the present invention. A vector suitable for cloning is referred to as “cloning vector”. Such a cloning vector ordinarily contains a multiple cloning site containing a plurality of restriction sites. At present, there are a number of vectors available for cloning genes in the art, which are designated different names by distribution sources depending on small differences (e.g., the type or sequence of a restriction enzyme for multicloning sites). For example, representative vectors are described in “Molecular Cloning (3rd edition)” by Sambrook, J. and Russell, D. W., Appendix 3 (Volume 3), Vectors and Bacterial strains. A3.2 (Cold Spring Harbor USA, 2001) (selling agencies are also described therein) and can be used as appropriate by those skilled in the art depending on the purpose.

As used herein, the term “expression vector” refers to a nucleic acid sequence comprising a structural gene and a promoter for regulating expression thereof, and in addition, various regulatory elements in a state that allows them to operate within host cells. The regulatory element may include, preferably, terminators, selectable markers such as drug-resistance genes, and enhancers.

Examples of a recombinant vector used herein include, but are not limited to, a lambda FIX vector (phage vector) for screening genome libraries, and a lambda ZAP vector (phage vector) for screening cDNA. For cloning genomic DNA, pBluescript II SK+/−, pGEM, and pCR2.1 vectors (plasmid vectors) can be mainly used. As an expression vector, a pSV2neo vector (plasmid vector) can be used. Such vectors can be used as appropriate with reference to Molecular Cloning A3.2 (supra).

As used herein, the term “terminator” refers to a sequence which is located downstream of a protein-encoding region of a gene and which is involved in the termination of transcription when DNA is transcribed into mRNA, and the addition of a poly-A sequence. It is known that a terminator contributes to the stability of mRNA, and has an influence on the amount of gene expression.

As used herein, the term “promoter” refers to a base sequence which determines the initiation site of transcription of a gene and is a DNA region which directly regulates the frequency of transcription. Transcription is started by RNA polymerase binding to a promoter. A promoter region is usually located within about 2 kbp upstream of the first exon of a putative protein coding region. Therefore, it is possible to estimate a promoter region by predicting a protein coding region in a genomic base sequence using DNA analysis software. A putative promoter region is usually located upstream of a structural gene, but depending on the structural gene, i.e., a putative promoter region may be located downstream of a structural gene. Preferably, a putative promoter region is located within about 2 kbp upstream of the translation initiation site of the first exon.

As used herein, the term “enhancer” refers to a sequence which is used so as to enhance the expression efficiency of a gene of interest. One or more enhancers may be used, or no enhancer may be used.

As used herein, the term “operatively linked” indicates that a desired sequence is located such that expression (operation) thereof is under control of a transcription and translation regulatory sequence (e.g., a promoter, an enhancer, and the like) or a translation regulatory sequence. In order for a promoter to be operatively linked to a gene, typically, the promoter is located immediately upstream of the gene. A promoter is not necessarily adjacent to a structural gene.

Any technique may be used herein for introduction of a nucleic acid molecule into cells, including, for example, transformation, transduction, transfection, and the like. Such a nucleic acid molecule introduction technique is well known in the art and commonly used, and is described in, for example, Ausubel F. A. et al., editors, (1988), Current Protocols in Molecular Biology, Wiley, New York, N.Y.; Sambrook J. et al. (1987) Molecular Cloning: A Laboratory Manual, 2nd Ed. and its 3rd Ed., Cold Spring Harbor Laboratory Press, Cold Spring Harbor, N.Y.; Special issue, Jikken Igaku [Experimental Medicine] Experimental Method for Gene introduction & Expression Analysis”, Yodo-sha, 1997; and the like. Gene introduction can be confirmed by method as described herein, such as Northern blotting analysis and Western blotting analysis, or other well-known, common techniques.

Any of the above-described methods for introducing DNA into cells can be used as an vector introduction method, including, for example, transfection, transduction, transformation, and the like (e.g., a calcium phosphate method, a liposome method, a DEAE dextran method, an electroporation method, a particle gun (gene gun) method, and the like).

As used herein, the term “transformant” refers to the whole or a part of an organism, such as a cell, which is produced by transformation. Examples of a transformant include a prokaryotic cell, yeast, an animal cell, a plant cell, an insect cell, and the like. Transformants may be referred to as transformed cells, transformed tissue, transformed hosts, or the like, depending on the subject. A cell used herein may be a transformant.

When a prokaryotic cell is used herein for genetic operations or the like, the prokaryotic cell may be of, for example, genus Escherichia, genus Serratia, genus Bacillus, genus Brevibacterium, genus Corynebacterium, genus Microbacterium, genus Pseudomonas, or the like. Specifically, the prokaryotic cell is, for example, Escherichia coli XL1-Blue, Escherichia coli XL2-Blue, Escherichia coli DH1, or the like. Such cells are described in, for example, “Molecular Cloning (3rd edition)” by Sambrook, J. and Russell, D. W., Appendix 3 (Volume 3), Vectors and Bacterial strains. A3.2 (Cold Spring Harbor USA 2001).

Examples of an animal cell as used herein include a mouse myeloma cell, a rat myeloma cell, a mouse hybridoma cell, a Chinese hamster ovary (CHO) cell, a baby hamster kidney (BHK) cell, an African green monkey kidney cell, a human leukemic cell, HBT5637 (Japanese Laid-Open Publication No. 63-299), a human colon cancer cell line, and the like. The mouse myeloma cell includes ps20, NSO, and the like. The rat myeloma cell includes YB2/0 and the like. A human embryo kidney cell includes HEK293 (ATCC:CRL-1573) and the like. The human leukemic cell includes BALL-1 and the like. The African green monkey kidney cell includes COS-1, COS-7, and the like. The human colon cancer cell line includes, but is not limited to, HCT-15, and the like, preferably, for example, Cos1, NIH3T3, and ES (R1, TMA, NR2) cells.

Any method for introduction of DNA can be used herein as a method for introduction of a recombinant vector, including, for example, a calcium chloride method, an electroporation method (Methods. Enzymol., 194, 182 (1990)), a lipofection method, a spheroplast method (Proc. Natl. Acad. Sci. USA, 84, 1929(1978)), a lithium acetate method (J. Bacteriol., 153, 163(1983)), a method described in Proc. Natl. Acad. Sci. USA, 75, 1929 (1978), and the like.

The transient expression of Cre enzyme, DNA mapping on a chromosome, and the like, which are used herein in a method for removing a genome, a gene locus, or the like, are well known in the art, as described in Kenichi Matsubara and Hiroshi Yoshikawa, editors, Saibo-Kogaku [Cell Engineering], special issue, “Experiment Protocol Series “FISH Experiment Protocol From Human Genome Analysis to Chrmosome/Gene diagnosis”, Shujun-sha (Tokyo), and the like.

Gene expression (e.g., mRNA expression, polypeptide expression) may be “detected” or “quantified” by an appropriate method, including mRNA measurement and immunological measurement method. Examples of the molecular biological measurement method include a Northern blotting method, a dot blotting method, a PCR method, and the like. Examples of the immunological measurement method include an ELISA method, an RIA method, a fluorescent antibody method, a Western blotting method, an immunohistological staining method, and the like, where a microtiter plate may be used. Examples of a quantification method include an ELISA method, an RIA method, and the like. A gene analysis method using an array (e.g., a DNA array, a protein array, etc.) may be used. The DNA array is widely reviewed in Saibo-Kogaku [Cell Engineering], special issue, “DNA Microarray and Up-to-date PCR Method”, edited by Shujun-sha. The protein array is described in detail in Nat Genet. 2002 December; 32 Suppl:526-32. Examples of a method for analyzing gene expression include, but are not limited to, an RT-PCR method, a RACE method, an SSCP method, an immunoprecipitation method, a two-hybrid system, an in vitro translation method, and the like in addition to the above-described techniques. Other analysis methods are described in, for example, “Genome Analysis Experimental Method, Yusuke Nakamura's Labo-Manual, edited by Yusuke Nakamura, Yodo-sha (2002), and the like. All of the above-described publications are herein incorporated by reference.

As used herein, the term “amount of expression” refers to the amount of a polypeptide or mRNA expressed in a subject cell. The amount of expression includes the amount of expression at the protein level of a polypeptide of the present invention evaluated by any appropriate method using an antibody of the present invention, including immunological measurement methods (e.g., an ELISA method, an RIA method, a fluorescent antibody method, a Western blotting method, an immunohistological staining method, and the like, or the amount of expression at the mRNA level of a polypeptide of the present invention evaluated by any appropriate method, including molecular biological measurement methods (e.g., a Northern blotting method, a dot blotting method, a PCR method, and the like). The term “change in the amount of expression” indicates that an increase or decrease in the amount of expression at the protein or mRNA level of a polypeptide of the present invention evaluated by an appropriate method including the above-described immunological measurement method or molecular biological measurement method.

(Polypeptide Production Method)

A transformant derived from a microorganism, an animal cell, or the like, which possesses a recombinant vector into which DNA encoding a polypeptide of the present invention is incorporated, is cultured according to an ordinary culture method. The polypeptide of the present invention is produced and accumulated. The polypeptide of the present invention is collected from the culture, thereby making it possible to produce the polypeptide of the present invention.

The transformant of the present invention can be cultured on a culture medium according to an ordinary method for use in culturing host cells. A culture medium for a transformant obtained from a prokaryote (e.g., E. coli) or a eukaryote (e.g., yeast) as a host may be either a naturally-occurring culture medium or a synthetic culture medium as long as the medium contains a carbon source, a nitrogen source, inorganic salts, and the like which an organism of the present invention can assimilate and the medium allows efficient culture of the transformant.

The carbon source includes any one that can be assimilated by the organism, such as carbohydrates (e.g., glucose, fructose, sucrose, molasses containing these, starch, starch hydrolysate, and the like), organic acids (e.g., acetic acid, propionic acid, and the like), alcohols (e.g., ethanol, propanol, and the like), and the like.

The nitrogen source includes ammonium salts of inorganic or organic acids (e.g., ammonia, ammonium chloride, ammonium sulfate, ammonium acetate, ammonium phosphate, and the like), and other nitrogen-containing substances (e.g., peptone, meat extract, yeast extract, corn steep liquor, casein hydrolysate, soybean cake, and soybean cake hydrolysate, various fermentation bacteria and digestion products thereof), and the like.

Salts of inorganic acids, such as potassium (I) phosphate, potassium (II) phosphate, magnesium phosphate, magnesium phosphate, sodium chloride, iron (I) sulfate, manganese sulfate, copper sulfate, calcium carbonate, and the like, can be used. Culture is performed under aerobic conditions for shaking culture, deep aeration agitation culture, or the like.

Culture temperature is preferably 15 to 40° C., culture time is ordinarily 5 hours to 7 days. The pH of culture medium is maintained at 3.0 to 9.0. The adjustment of pH is carried out using inorganic or organic acid, alkali solution, urea, calcium carbonate, ammonia, or the like. An antibiotic, such as ampicillin, tetracycline, or the like, may be optionally added to culture medium during cultivation.

When culturing a microorganism which has been transformed using an expression vector containing an inducible promoter, culture medium may be optionally supplemented with an inducer. For example, when a microorganism, which has been transformed using an expression vector containing a lac promoter, is cultured, isopropyl-β-D-thiogalactopyranoside or the like may be added to the culture medium. When a microorganism, which has been transformed using an expression vector containing a trp promoter, is cultured, indole acrylic acid or the like may be added to culture medium. A cell or an organ into which a gene has been introduced can be cultured in a large volume using a jar fermenter. Examples of a medium for culture include, but are not limited to, commonly used Murashige and Skoog (MS) medium, White medium, or these media supplemented with plant hormones, such as auxin and cytokinins.

For example, when an animal cell is used, a culture medium of the present invention for culturing the cell includes a commonly used RPMI1640 culture medium (The Journal of the American Medical Association, 199, 519 (1967)), Eagle's MEM culture medium (Science, 122, 501(1952)), DMEM culture medium (Virology, 8, 396 (1959)), 199 culture medium (Proceedings of the Society for the Biological Medicine, 73, 1 (1950)) or these culture media supplemented with fetal bovine serum or the like.

Culture is normally carried out for 1 to 7 days under conditions such as pH 6 to 8, 25 to 40° C., 5% CO₂. An antibiotic, such as kanamycin, penicillin, streptomycin, or the like may be optionally added to culture medium during cultivation.

A polypeptide of the present invention can be isolated or purified from a culture of a transformant, which has been transformed with a nucleic acid sequence encoding the polypeptide, using an ordinary method for isolating or purifying enzymes, which are well known and commonly used in the art. For example, when a polypeptide of the present invention is secreted outside a transformant for producing the polypeptide, the culture is subjected to centrifugation or the like to obtain a soluble fraction. A purified specimen can be obtained from the soluble fraction by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Kasei Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.).

When a polypeptide of the present invention is accumulated in a dissolved form within a transformant cell for producing the polypeptide, the culture is subjected to centrifugation to collect cells in the culture. The cells are washed, followed by pulverization of the cells using a ultrasonic pulverizer, a French press, MANTON GAULIN homogenizer, Dinomil, or the like, to obtain a cell-free extract solution. A purified specimen can be obtained from a supernatant obtained by centrifuging the cell-free extract solution or by a technique, such as solvent extraction, salting-out/desalting with ammonium sulfate or the like, precipitation with organic solvent, anion exchange chromatography with a resin (e.g., diethylaminoethyl (DEAE)-Sepharose, DIAION HPA-75 (Mitsubishi Kasei Corporation), etc.), cation exchange chromatography with a resin (e.g., S-Sepharose FF (Pharmacia), etc.), hydrophobic chromatography with a resin (e.g., buthylsepharose, phenylsepharose, etc.), gel filtration with a molecular sieve, affinity chromatography, chromatofocusing, electrophoresis (e.g., isoelectric focusing electrophoresis, etc.).

When the polypeptide of the present invention has been expressed and formed insoluble bodies within cells, the cells are harvested, pulverized, and centrifuged. From the resulting precipitate fraction, the polypeptide of the present invention is collected using a commonly used method. The insoluble polypeptide is solubilized using a polypeptide denaturant. The resulting solubilized solution is diluted or dialyzed into a denaturant-free solution or a dilute solution, where the concentration of the polypeptide denaturant is too low to denature the polypeptide. The polypeptide of the present invention is allowed to form a normal three-dimensional structure, and the purified specimen is obtained by isolation and purification as described above.

Purification can be carried out in accordance with a commonly used protein purification method (J. Evan. Sadler et al.: Methods in Enzymology, 83, 458). Alternatively, the polypeptide of the present invention can be fused with other proteins to produce a fusion protein, and the fusion protein can be purified using affinity chromatography using a substance having affinity to the fusion protein (Akio Yamakawa, Experimental Medicine, 13, 469-474 (1995)). For example, in accordance with a method described in Lowe et al., Proc. Natl. Acad. Sci., USA, 86, 8227-8231 (1989)., Genes Develop., 4, 1288(1990)), a fusion protein of the polypeptide of the present invention with protein A is produced, followed by purification with affinity chromatography using immunoglobulin G.

A fusion protein of the polypeptide of the present invention with a FLAG peptide is produced, followed by purification with affinity chromatography using anti-FLAG antibodies (Proc. Natl. Acad. Sci., USA, 86, 8227(1989), Genes Develop., 4, 1288 (1990)).

The polypeptide of the present invention can be purified with affinity chromatography using antibodies which bind to the polypeptide. The polypeptide of the present invention can be produced using an in vitro transcription/translation system in accordance with a known method (J. Biomolecular NMR, 6, 129-134; Science, 242, 1162-1164; J. Biochem., 110, 166-168 (1991)).

Based on the amino acid information of a polypeptide as obtained above, the polypeptide can also be produced by a chemical synthesis method, such as the Fmoc method (fluorenylmethyloxycarbonyl method), the tBoc method (t-buthyloxycarbonyl method), or the like. The peptide can be chemically synthesized using a peptide synthesizer (manufactured by Advanced ChemTech, Applied Biosystems, Pharmacia Biotech, Protein Technology instrument, Synthecell-Vega, PerSeptive, Shimazu, or the like).

The structure of the purified polypeptide of the present invention can be carried out by methods commonly used in protein chemistry (see, for example, Hisashi Hirano. “Protein Structure Analysis for Gene Cloning”, published by Tokyo Kagaku Dojin, 1993). The physiological activity of a polypeptide of the present invention can be measured in accordance with a known measurement method.

(Method for Producing Mutant Polypeptide)

Amino acid deletion, substitution or addition of the polypeptide of the present invention can be carried out by a site-specific mutagenesis method which is a well known technique. One or several amino acid deletions, substitutions or additions can be carried out in accordance with methods described in Molecular Cloning, A Laboratory Manual, Second Edition, Cold Spring Harbor Laboratory Press (1989); Current Protocols in Molecular Biology, Supplement 1 to 38, John Wiley & Sons (1987-1997); Nucleic Acids Research, 10, 6487 (1982); Proc. Natl. Acad. Sci., USA, 79, 6409 (1982); Gene, 34, 315 (1985); Nucleic Acids Research, 13, 4431 (1985); Proc. Natl. Acad. Sci. USA, 82, 488 (1985); Proc. Natl. Acad. Sci., USA, 81, 5662 (1984); Science, 224, 1431 (1984); PCT WO85/00817(1985); Nature, 316, 601 (1985); and the like.

(Immunochemistry)

Preparation of antibodies which recognize the polypeptide of the present invention are also well known in the art. For example, preparation of polyclonal antibodies can be carried out by administering a purified specimen of the whole or a partial fragment of an obtained polypeptide or a peptide having a part of the amino acid sequence of the protein of the present invention, as an antigen, to an animal.

To produce antibodies, a rabbit, a goat, a rat, a mouse, a hamster, or the like can be used as an animal to which an antigen is administered. The dose of the antigen is preferably 50 to 100 μg per animal. When a peptide is used as an antigen, the peptide is preferably coupled via covalent bond to a carrier protein, such as keyhole limpet haemocyanin, bovine thyroglobulin, or the like. A peptide used as an antigen can be synthesized using a peptide synthesizer. The antigen is administered every 1 to 2 weeks after a first administration a total 3 to 10 times. 3 to 7 days after each administration, blood is collected from the venous plexus of eye grounds, and whether or not the serum reacts with the antigen which has been used for immunization is determined by an enzyme immunoassay (Enzyme immunoassay (ELISA): published by Igaku-syoin 1976; Antibodies—A Laboratory Manual, Cold Spring Harbor Laboratory (1988); and the like).

Serum is obtained from a non-human mammal whose serum exhibits a sufficient antibody titer to an antigen. From the serum, polyclonal antibodies can be isolated and purified using well known techniques. Production of monoclonal antibodies is also well known in the art. In order to prepare antibody secreting cells, a rat whose serum exhibits a sufficient antibody titer for fragments of a polypeptide of the present invention which has been used for immunization, is used as a source for antibody secreting cells, which are fused with myeloma cells to prepare hybridomas. Thereafter, a hybridoma specifically reacting with the fragments of the polypeptide of the present invention is selected using enzyme immunoassays. A monoclonal antibody secreted by the thus obtained hybridoma can be used for various purposes.

Such an antibody can be used for an immunological method of detecting the polypeptide of the present invention, for example. Examples of an immunological method of detecting the polypeptide of the present invention using the antibody of the present invention include an ELISA method using microtiter plates, a fluorescent antibody method, a Western blotting method, an immunohistological method, and the like.

Further, the antibody of the present invention can be used for immunological methods for quantifying the polypeptide of the present invention polypeptide. Examples of the immunological methods for quantifying the polypeptide of the present invention include a sandwich ELISA method using two monoclonal antibodies for different epitopes of the polypeptide of the present invention, which react with the polypeptide of the present invention; a radioimmunoassay using the polypeptide of the present invention labeled with a radioactive isotope, such as ¹²⁶i or the like, and antibodies which recognize the polypeptide of the present invention; and the like.

Methods for quantifying mRNA for the polypeptide of the present invention polypeptide are well known in the art. For example, the above-described oligonucleotides prepared from the polynucleotide or DNA of the present invention can be used to quantify the amount of expression of DNA encoding the polypeptide of the present invention based on the mRNA level using Northern hybridization or PCR. Such a technique is well known in the art and is described in literature described herein.

The polynucleotides may be obtained, and the nucleotide sequence of the polynucleotides determined, by any method known in the art. For example, if the nucleotide sequence of an antibody is known, a polynucleotide encoding the antibody may be assembled from chemically synthesized oligonucleotides (e.g., as described in Kutmeier et al., BioTechniques, 17: 242 (1994)), which, briefly, involves the synthesis of overlapping oligonucleotides containing portions of the sequence encoding the antibody, annealing and ligation of those oligonucleotides, and then amplification of the ligated oligonucleotides by PCR.

Alternatively, a polynucleotide encoding an antibody can be produced from a nucleic acid from a suitable source. If a clone containing a nucleic acid encoding a particular antibody is not available, but the sequence of the antibody molecule is known, a nucleic acid encoding the immunoglobulin may be obtained from a suitable source (e.g., an antibody cDNA library, or a cDNA library generated from any tissue or cells expressing the antibody (e.g., hybridoma cells selected to express an antibody of the present invention), or nucleic acids (preferably poly-A+RNA) isolated therefrom) by PCR amplification using synthetic primers hybridizable to the 3′ and 5′ ends of the sequence or by cloning using an oligonucleotide probe specific for the particular gene sequence to identify, for example, a cDNA clone from a cDNA library that encodes the antibody. Amplified nucleic acids produced by PCR may be cloned into replicable cloning vectors using any method well known in the art.

Once the nucleotide sequence and corresponding amino acid sequence of an antibody is determined, the nucleotide sequence of the antibody may be manipulated using methods well known in the art for the manipulation of nucleotide sequences (e.g., recombinant DNA techniques, site directed mutagenesis, PCR, etc. (see, for example, the techniques described in Sambrook et al., 1990, Molecular Cloning, A Laboratory Manual, 2nd Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to produce antibodies having a different amino acid sequence, for example, to create amino acid substitutions, deletions, and/or insertions.

In a specific embodiment, the amino acid sequence of heavy and/or light chain variable domains may be inspected to identify the sequences of the complementarity determining regions (CDRs) by methods that are well know in the art (e.g., by comparison to known amino acid sequences of other heavy and light chain variable regions to determine the regions of sequence hypervariability). Using routine recombinant DNA techniques, one or more of the CDRs may be inserted within framework regions (e.g., into human framework regions to humanize a non-human antibody) as described above. The framework regions may be naturally occurring or consensus framework regions, and preferably human framework regions (see, e.g., Chothia et al., J. Mol. Biol. 278: 457-479 (1998) for a listing of human framework regions). Preferably, the polynucleotide generated by the combination of the framework regions and CDRs encodes an antibody that specifically binds a polypeptide of the present invention. Preferably, as discussed above, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314: 452-454) by splicing genes from a mouse antibody molecule of appropriate antigen specificity together with genes from a human antibody molecule of appropriate biological activity can be used. As described above, a chimeric antibody is a molecule in which different portions are derived from different animal species. Such a molecule has a variable region derived from a murine mAb and a human immunoglobulin constant region (e.g., humanized antibodies).

Known techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

(Methods of Producing Antibodies)

The antibodies of the present invention can be produced by any method known in the art for the synthesis of antibodies, by chemical synthesis, or preferably, by recombinant expression techniques.

Recombinant expression of an antibody of the present invention, or fragment, derivative or analog thereof (e.g., a heavy or light chain of an antibody of the present invention) requires construction of an expression vector containing a polynucleotide that encodes the antibody. Once a polynucleotide encoding an antibody molecule or a heavy or light chain of an antibody, or portion thereof (preferably containing the heavy or light chain variable domain), of the present invention has been obtained, a vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art may be used to construct expression vectors containing antibody coding sequences and appropriate transcriptional and translational control signals. These methods include, for example, in vitro recombinant DNA techniques, synthetic techniques, and in vivo genetic recombination. The present invention, thus, provides replicable vectors comprising a nucleotide sequence encoding an antibody molecule of the present invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the present invention. Thus, the present invention includes host cells containing a polynucleotide encoding an antibody of the present invention, or a heavy or light chain thereof, operably linked to a heterologous promoter. In preferred embodiments for the expression of double-chained antibodies, vectors encoding both the heavy and light chains may be co-expressed in the host cell for expression of the entire immunoglobulin molecule, as detailed below.

(Screening)

As used herein, the term “screening” refers to selection of a target, such as an organism, a substance, or the like, a given specific property of interest from a population containing a number of elements using a specific operation/evaluation method. For screening, an agent (e.g., an antibody), a polypeptide or a nucleic acid molecule of the present invention can be used.

As used herein, screening by utilizing an immunological reaction is also referred to as “immunophenotyping”. In this case, an antibody or a single chain antibody of the present invention may be used for immunophenotyping a cell line and a biological sample. A transcription or translation product of a gene of the present invention may be useful as a cell specific marker, or more particularly, a cell marker which is distinctively expressed in various stages in differentiation and/or maturation of a specific cell type. A monoclonal antibody directed to a specific epitope, or a combination of epitopes allows for screening of a cell population expressing a marker. Various techniques employ monoclonal antibodies to screen for a cell population expressing a marker. Examples of such techniques include, but are not limited to, magnetic separation using magnetic beads coated with antibodies, “panning” using antibodies attached to a solid matrix (i.e., a plate), flow cytometry, and the like (e.g., U.S. Pat. No. 5,985,660; and Morrison et al., Cell, 96:737-49(1999)).

These techniques may be used to screen cell populations containing undifferentiated cells, which can grow and/or differentiate as seen in human umbilical cord blood or which are treated and modified into an undifferentiated state (e.g., embryonic stem cells, tissue stem cells, etc.).

(Gene Therapy)

In an embodiment of the present invention, a nucleic acid comprising a sequence encoding an antibody or a functional derivative thereof is administered for the purpose of gene therapy for treatment, inhibition, or prophylaxis of a disease or a disorder associated with abnormal expression and/or activity of a polypeptide of the present invention. Gene therapy means that subjects are treated by administering an expressed or expressible nucleic acid thereto. In this embodiment of the present invention, a protein encoded by a nucleic acid is produced and the protein mediates a therapeutic effect.

Any technique available in the art for gene therapy may be employed in the present invention. Illustrative techniques are described as follows.

Gene therapy techniques are generally reviewed in, for example, Goldspiel et al., Clinical Pharmacy 12: 488-505(1993); Wu and Wu, Biotherapy 3: 87-95(1991); Tolstoshev, Ann. Rev. Pharmacol. Toxicol., 32: 573-596(1993); Mulligan, Science 260: 926-932(1993); and Morgan and Anderson, Ann. Rev. Biochem., 62: 191-217(1993); May, TIBTECH 11(5): 155-215(1993). Recombinant DNA techniques generally known, which are generally used in gene therapy, are described in, for example, Ausubel et al. (ed.), Current Protocols in Molecular Biology, John Wiley & Sons, NY (1993); and Kriegler, Gene Transfer and Expression, A Laboratory Manual, Stockton Press, NY (1990).

(Demonstration of Therapeutic Activity or Prophylactic Activity)

The compounds or pharmaceutical compositions of the present invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art (including, but not limited to, cell lysis assays). In accordance with the present invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

(Therapeutic/Prophylactic Administration and Composition)

The present invention provides methods of treatment, inhibition and prophylaxis by administration to a subject of an effective amount of a compound or pharmaceutical composition of the present invention. In a preferred aspect, the compound is substantially purified (e.g., substantially free from substances that limit its effect or produce undesired side-effects). Preferable examples of a subject include, but are not limited to, animals, such as cattle, pig, horse, chicken, cat, dog, and the like, more preferably mammal, and most preferably human.

When a nucleic acid molecule or polypeptide of the present invention is used as a medicament, the medicament may further comprise a pharmaceutically acceptable carrier. Any pharmaceutically acceptable carrier known in the art may be used in the medicament of the present invention.

Examples of a pharmaceutical acceptable carrier or a suitable formulation material include, but are not limited to, antioxidants, preservatives, colorants, flavoring agents, diluents, emulsifiers, suspending agents, solvents, fillers, bulky agents, buffers, delivery vehicles, and/or pharmaceutical adjuvants. Represetatively, a medicament of the present invention is administered in the form of a composition comprising an isolated pluripotent stem cell, or a variant or derivative thereof, with at least one physiologically acceptable carrier, exipient or diluent. For example, an appropriate vehicle may be injection solution, physiological solution, or artificial cerebrospinal fluid, which can be supplemented with other substances which are commonly used for compositions for parenteral delivery.

Acceptable carriers, excipients or stabilizers used herein preferably are nontoxic to recipients and are preferably inert at the dosages and concentrations employed, and preferably include phosphate, citrate, or other organic acids; ascorbic acid, α-tocopherol; low molecular weight polypeptides; proteins (e.g., serum albumin, gelatin, or immunoglobulins); hydrophilic polymers (e.g., polyvinylpyrrolidone); amino acids (e.g., glycine, glutamine, asparagine, arginine or lysine); monosaccharides, disaccharides, and other carbohydrates (glucose, mannose, or dextrins); chelating agents (e.g., EDTA); sugar alcohols (e.g., mannitol or sorbitol); salt-forming counterions (e.g., sodium); and/or nonionic surfactants (e.g., Tween, pluronics or polyethylene glycol (PEG)).

Examples of appropriate carriers include neutral buffered saline or saline mixed with serum albumin. Preferably, the product is formulated as a lyophilizate using appropriate excipients (e.g., sucrose). Other standard carriers, diluents, and excipients may be included as desired. Other exemplary compositions comprise Tris buffer of about pH 7.0-8.5, or acetate buffer of about pH 4.0-5.5, which may further include sorbitol or a suitable substitute therefor.

The medicament of the present invention may be administered orally or parenterally. Alternatively, the medicament of the present invention may be administered intravenously or subcutaneously. When systemically administered, the medicament for use in the present invention may be in the form of a pyrogen-free, pharmaceutically acceptable aqueous solution. The preparation of such pharmaceutically acceptable compositions, with due regard to pH, isotonicity, stability and the like, is within the skill of the art. Administration methods may be herein oral, parenteral administration (e.g., intravenous, intramuscular, subcutaneous, intradermal, to mucosa, intrarectal, vaginal, topical to an affected site, to the skin, etc.). A prescription for such administration may be provided in any formulation form. Such a formulation form includes liquid formulations, injections, sustained preparations, and the like.

The medicament of the present invention may be prepared for storage by mixing a sugar chain composition having the desired degree of purity with optional physiologically acceptable carriers, excipients, or stabilizers (Japanese Pharmacopeia ver. 14, or a supplement thereto or the latest version; Remington's Pharmaceutical Sciences, 18th Edition, A. R. Gennaro, ed., Mack Publishing Company, 1990; and the like), in the form of lyophilized cake or aqueous solutions.

The amount of the composition of the present invention used in the treatment method of the present invention can be easily determined by those skilled in the art with reference to the purpose of use, a target disease (type, severity, and the like), the patient's age, weight, sex, and case history, the form or type of the cell, and the like. The frequency of the treatment method of the present invention applied to a subject (or patient) is also determined by those skilled in the art with respect to the purpose of use, target disease (type, severity, and the like), the patient's age, weight, sex, and case history, the progression of the therapy, and the like. Examples of the frequency include once per day to several months (e.g., once per week to once per month). Preferably, administration is performed once per week to month with reference to the progression.

(Reprogramming)

As used herein, the term “reprogramming” means that a cell (e.g., a somatic cell) is caused to be in the undifferentiated state so that the cell increases or acquires pluripotency. Therefore, reprogramming activity may be measured as follows, for example. A differentiated cell (e.g., a somatic cell, etc.) is exposed to a predetermined amount of a certain agent for a predetermined period of time (e.g., several hours, etc.). Thereafter, the pluripotency of the cell is measured and compared with the pluripotency of the cell before exposure. By determining whether or not a significant difference is found, the reprogramming activity is determined. There are various reprogrammed levels, which correspond to the pluripotency levels of a reprogrammed cell. Therefore, when a reprogramming agent derived from a totipotent stem cell is used, reprogramming may correspond to imparting totipotency. Therefore, herein, a reprogram state and an undifferentiated state have substantially one-to-one correspondence.

As used herein, the term “reprogramming agent” refers to an agent which acts on cells to cause the cells to be in the undifferentiated state. Embryonic stem cells cannot reprogram imprints in the nuclei of somatic cells, and can reprogram the epigenetic state of the nuclei of somatic cells so that germ cells can be developed. Therefore, it is clear that embryonic stem cells have an agent capable of reprogramming. There is also a possibility that stem cells other than embryonic stem cells possess an agent capable of reprogramming somatic cells. Such a reprogramming agent is also encompassed by the present invention. Examples of an embryonic stem cell-derived component which is applied to somatic cells include, but are not limited to, components contained in embryonic stem cells, including cytoplasmic components, nuclear components, individual RNAs and proteins, and the like. When cytoplasmic or nuclear components including miscellaneous molecules are applied, the components may be fractioned to some degree with a commonly used technique (e.g., chromatography, etc.), and each fraction may be applied to somatic cells. If a specific fraction is revealed to contain a reprogramming agent, the fraction can be further purified so that a single molecule is eventually specified and such a molecule can be used. Alternatively, a fraction containing a reprogramming agent can be used without any purification to reprogram somatic cells. It may be considered that a single molecule achieves reprogramming. Alternatively, it may be considered that a plurality of molecules interact one another to alter somatic cells into the undifferentiated state. Therefore, the “reprogramming agent” of the present invention includes an agent consisting of a single molecule, an agent consisting of a plurality of molecules, and a composition comprising the single molecule or the plurality of molecules.

A reprogramming agent of the present invention can be screened for as follows. Components derived from embryonic stem cells are caused to act on somatic cells by means of contact, injection, or the like. The action is detected based on the expression of a Stm gene-GFP marker gene of the present invention, the activation of the X chromosome, or the like, as an indicator for reprogramming. A component having reprogramming activity is selected.

A “reprogramming agent contained in an embryonic stem cell” of the present invention can be obtained by a screening method as described above. The reprogramming agent may be an enzyme for methylation of histone H3-Lys4 or an agent which is involved in the methylation. There is a possibility that such a component is contained in cells (e.g., tissue stem cells, etc.) other than embryonic stem cells. However, once a reprogramming agent is identified from an embryonic stem cell by the above-described method, such are programming agent can be obtained or produced from other materials based on the identified reprogramming agent. For example, if a reprogramming agent obtained by the above-described method is RNA, the RNA can be sequenced and RNA having the same sequence can be synthesized using a well-known technique. Alternatively, if a reprogramming agent is a protein, antibodies for the protein are produced and the ability of the antibodies to the protein can be utilized to obtain the reprogramming agent from materials which contain the agent. Alternatively, the amino acid sequence of the protein is partially determined; a probe hybridizable to a gene encoding the partial amino acid sequence is produced; and cDNA and genomic DNA encoding the protein can be obtained by a hybridization technique. Such a gene can be amplified by PCR, though a primer needs to be prepared. A gene encoding a reprogramming agent obtained by any of the above-described methods can be used to produce the reprogramming agent by a well-known gene recombinant technique. Therefore, a “reprogramming agent contained in an embryonic stem cell” of the present invention is not necessarily obtained from embryonic stem cells and can be obtained from cells having pluripotency (e.g., tissue stem cells, etc.). Therefore, the reprogramming agent includes all agents capable of reprogramming a somatic cell.

A reprogramming agent may be obtained by the following screening method. Embryonic stem cell-derived components are caused to act on an appropriate somatic cell. A component having an activity to reprogram the somatic cell is selected by detecting the activity. Illustrative examples of a somatic cell used herein include, but are not limited to, lymphocytes, spleen cells, testis-derived cells, and the like. Any somatic cells can be used, which have normal chromosomes, can be stably grown, and can be altered by action of a reprogramming agent into an undifferentiated cell having pluripotency. Particularly, it is preferable that a somatic cell used for screening is derived from the same species as that of an embryonic stem cell from which components are collected (e.g., a human-derived somatic cell when an embryonic stem cell is derived from a human). Previously established cell lines can be used.

In a method for producing a cell, a tissue, or an organ from a cell of the present invention, the cell is differentiated by a method which is not particularly limited as long as the cell is differentiated into a cell, a tissue or an organ, while the karyotype of the cell is substantially retained. For example, by introducing a cell into a blastocyst, subcutaneously injecting a cell into an animal (e.g., a mouse, etc.) to form a teratoma, or the like, the cell can be differentiated into a cell, a tissue, and an organ. A desired cell, tissue, or organ can be isolated from the differentiated blastocyst or teratoma. A desired cell, tissue, or organ may be induced in vitro from a cell by adding a cell growth factor, a growth factor, or the like which is required for obtaining a cell of the type of interest. To date there have been reports for induction of blood vessel, neuron, muscle cell, hematopoietic cell, skin, bone, liver, pancreas, or the like from embryonic stem cells. These techniques can be applied when a cell, tissue, or organ corresponding to an implantation recipient is produced from a pluripotent stem cell according to the present invention (e.g., Kaufman, D. S., Hanson, E. T., Lewis, R. L., Auerbach, R., and Thomson, J. A. (2001), Proc. Natl. Acad. Sci. USA., 98, 10716-21; Boheler, K. R., Czyz, J., Tweedie, D., Yang, H. T., Anisimov, S. V., and Wobus, A. M. (2002), Circ. Res., 91, 189-201).

When a stem cell (e.g., an embryonic stem cell, etc.) is used in a method for producing a cell, a tissue, or an organ from a cell according to the present invention, the stem cell can be established from an appropriate individual stem cell (e.g., a neural stem cell, an embryonic stem cell, etc.), or previously established stem cells (e.g., neural stem cells, embryonic stem cells, etc.) derived from various organisms are preferably utilized. For example, examples of such a stem cell include, but are not limited to, stem cells (e.g., embryonic stem cells, etc.) of mouse, hamster, pig, sheep, bovine, mink, rabbit, primate (e.g., rhesus monkey, marmoset, human, etc.), and the like. Preferably, stem cells (e.g., embryonic stem cells, etc.) derived from the sample species as that of somatic cells of interest are employed.

(Description of Stm Genes)

Embryonic stem (ES) cells derived from early embryos and embryonic germ (EG) cells derived primordial germ cells were compared in mRNA to identify a gene which was highly expressed in both cells. The base sequence of cDNA of the novel gene was determined. The structure of the gene was determined in the mouse genome. According to the result of Southern hybridization analysis, it was inferred that mouse has 4 homologous genes. The base sequence of cDNA of at least one of the homologous genes has been clarified by database search using the base sequence of cDNA. In addition, according to the result of the search on a human database, it was inferred that the four homologous genes were present on the human genome.

For analysis of the expression pattern of Stm genes, total RNA were collected from early embryos and germ cells, followed by RT-PCR analysis. Whereas the Stm gene was not expressed in 12.5-day-old embryos, the expression was observed in female and male gonads. In addition, whereas the expression was not detected in unfertilized eggs, the expression was detected from blastocysts to 7.5-day-old embryos. The expression was suppressed in embryos on more subsequent developmental stages. These results show that the Stm gene is expressed specifically in undifferentiated cells. Comparing with another undifferentiation-specific expression gene Oct3/4, it was shown that the Stm gene has a different expression pattern at least in unfertilized eggs. RT-PCR and Northern hybridization revealed a high level of expression in embryonic stem cells. For the purpose of determining an expression site of the Stm gene in early embryos, an attempt is being made to introduce a reporter gene under the control of the Stm gene.

The Stm gene is applied to the following clinical applications, for example. A pluripotent cell, such as an embryonic stem cell, a tissue stem cell, or the like, is differentiated into a specific tissue cell, which is in turn implanted into a site having an impaired function. In this case, the implanted cell substitutes for the impaired function. Such regenerative medicine has attracted attention as a near-future therapy. A plurality of marker molecules are required for confirming that the undifferentiated state of embryonic stem cells and other pluripotent stem cells is maintained. The Stm gene is optimally suitable as a marker gene for undifferentiated cells. A great deal of attention has been focused on the elucidation of a mechanism and an agent for producing pluripotent cells, which can be applied to regenerative medicine, by reprogramming somatic cells of individuals. To reveal the mechanism for reprogramming the nucleus of a somatic cell, it is necessary to use a plurality of undifferentiated state-specific markers to know to what degree the somatic cell is reprogrammed. The Stm gene has a potential to play an important role as a marker for reprogramming.

The present invention revealed that the Stm gene is different from Oct3/4 in a number of points, though the Stm gene has a similar pattern of gene expression pattern. In addition, it is expected that the Stm gene has a homeobox and functions as a transcription agent. Alternatively, attention has also been focused on the association with the size of a telomere and the association with β galactosidase activity as a cell aging marker. In fact, the present inventors provided data suggesting the localization of the Stm gene in nuclei, which certainly demonstrates that the Stm gene plays an important role in maintenance of an undifferentiated state. Therefore, a STM protein may be useful as a novel drug for rejuvenating cells or as a tool for screening for such a drug.

(Description of Preferred Embodiments)

Hereinafter, preferred embodiments of the present invention will be described. The following embodiments are provided for a better understanding of the present invention and the scope of the present invention should not be limited to the following description. It will be clearly appreciated by those skilled in the art that variations and modifications can be made without departing from the scope of the present invention with reference to the specification.

(Stm Gene in Nucleic Acid Form)

Therefore, according to one aspect, the present invention relates to a Stm gene. Such a Stm gene may be a nucleic acid molecule, comprising:

(a) a polynucleotide having a base sequence set forth in SEQ ID NO. 1, 3, 5 or 29, or a fragment thereof;

(b) a polynucleotide encoding a polypeptide consisting of an amino acid sequence set forth in SEQ ID NO. 2, 4, 6 or 30, or a fragment thereof;

(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;

(d) a polynucleotide, which is a spliced mutant or alleic mutant of a base sequence set forth in SEQ ID NO. 1, 3, 5 or 29, or a fragment thereof;

(e) a polynucleotide encoding a species homolog of a polypeptide consisting of an amino acid sequence set forth in SEQ ID NO. 2, 4, 6 or 30, or a fragment thereof;

(f) a polynucleotide hybridizable to any one of the polynucleotides of (a) to (e) under stringent conditions and encoding a polypeptide having biological activity; or

(g) a polynucleotide consisting of a base sequence having at least 70% identity to any one of the polynucleotides (a) to (e) or a complementary sequence thereof, and encoding a polypeptide having biological activity.

In one preferred embodiment, the number of substitutions, additions, and deletions in (c) is preferably limited to, for example, 50 or less, 40 or less, 30 or less, 20 or less, 15 or less, 10 or less, 9 or less, 8 or less, 7 or less, 6 or less, 5 or less, 4 or less, 3 or less, or 2 or less. The lesser number of substitutions, additions, and deletions is more preferable. However, such a number may be great as long as the Stm gene holds biological activity (preferably, the product of the Stm gene is similar to the product of the Stm1 gene or the Stm gene has substantially the same activity as that of the Stm1 gene).

In another preferred embodiment, the above-described variant polypeptide has biological activity, such as, for example, interaction with antibodies specific to a polypeptide consisting of an amino acid sequence set forth in SEQ ID NO. 2, 4, 6 or 30 or a fragment thereof, maintenance of an undifferentiated state, and the like. The present invention is not limited to this. Preferably, such biological activity includes maintenance of an undifferentiated state. Stm is considered to play an important role in the maintenance of an undifferentiated state of cells. Specifically, since the Stm gene has a homeodomain, it is inferred that the Stm gene suppresses expression of a downstream gene which, for example, induces differentiation of a tissue cell. It is considered that such activity can be measured by gene deletion experiments, RNAi experiments, experiments of inhibiting the function of a protein using antibodies, or the like.

In another preferred embodiment, the alleic mutant in (d) preferably has at least 90% homology to a nucleic acid sequence set forth in SEQ ID NO. 1, 3, 5 or 29 in the same variety or strain, or the like, for example, such an alleic mutant preferably has at least 99% homology, and even more preferably at least 99.7%. Particularly, the alleic mutant preferably maintains the difference between the Stm1 gene and the Stm2 gene.

The above-described species homologs can be identified by searching a gene sequence database of the species, if any, using the Stm gene of the present invention as a query sequence. Alternatively, the species homologs can be identified by screening a gene library of the species using the whole or a part of the Stm gene of the present invention as a probe or a primer. Such identifying methods are well known in the art and are also described in documents mentioned herein. The species homolog preferably has at least about 30% homology to, for example, a nucleic acid sequence set forth in SEQ ID NO. 1, 3, 5 or 29. The species homolog preferably has at least about 50% homology to a nucleic acid sequence set forth in SEQ ID NO. 1, 3, 5 or 29.

In a preferred embodiment, the identity to any one of the above-described polynucleotides (a) to (e) or a complementary sequence thereof may be at least about 80%, more preferably at least about 90%, even more preferably at least about 98%, and most preferably at least about 99%.

In a preferred embodiment, a nucleic acid molecule of the present invention may have at least 8 contiguous nucleotides. A nucleic acid molecule of the present invention may have appropriate nucleotides in length which varies depending on the purpose of the present invention. More preferably, a nucleic acid molecule of the present invention may have at least 10 contiguous nucleotides in length, more preferably at least 15 contiguous nucleotides in length, and even more preferably at least 20 contiguous nucleotides in length. The lower limit of these nucleotide lengths may include values specifically described herein and values therebetween (e.g., 9, 11, 12, 13, 14, 16, etc.) or values more than those values (e.g., 21, 22, . . . , 30, etc.). The length of a nucleic acid molecule of the present invention may have an upper limit which is the full length of a sequence set forth in SEQ ID NO. 1, 3, 5 or 29 or more than the full length as long as it can be used in an application of interest (e.g., a marker). Alternatively, when used as a primer, a nucleic acid molecule of the present invention may typically have at least about 8 nucleotides in length, and preferably about 10 nucleotides in length. When used as a probe, a nucleic acid molecule of the present invention may typically have at least about 15 nucleotides in length, and preferably about 17 nucleotides in length.

In a more preferred embodiment, the present invention may provide a polynucleotide encoding (a) a polypeptide having a base sequence set forth in SEQ ID NO. 1, 3, 5 or 29 or a fragment thereof; or (b) a polypeptide consisting of an amino acid sequence of SEQ ID NO. 2, 4, 6 or 30 or a fragment thereof.

In a certain preferred embodiment, a nucleic acid molecule of the present invention comprises:

(a) a polynucleotide having a base sequence of positions 1037 to 1607 or 244 to 1126 set forth in SEQ ID NO. 3 or a base sequence in corresponding positions, or a fragment thereof;

(b) a polynucleotide hybridizable to the polynucleotide of (a) under stringent conditions, and encoding a polypeptide biological activity; or

(c) a polynucleotide consisting of a base sequence having at least 70% identity to any one of the polynucleotides of (a) to (b) or a complementary sequence thereof, and encoding a polypeptide having biological activity.

In this case, a sequence capable of being used as a Stm marker is, for example, a region between positions 1037-1056 (F1 primer) and positions 1607-1587 (R1 primer) in SEQ ID NO. 3, a region between positions 244-253(F2 primer) and positions 1126-1107 (R2 primer) in SEQ ID NO. 3, and positions corresponding to genes corresponding to these regions (e.g., a gene encoded by a sequence set forth in SEQ ID NO. 1 or 5, or SEQ ID NO. 7 or 9).

In a more preferred embodiment, the identity to any one of the above-described polynucleotides (a) to (b) or a complementary sequence thereof may be at least about 80%, more preferably at least about 90%, even more preferably at least about 98%, and most preferably at least about 99%.

In a preferred embodiment, a nucleic acid molecule encoding the Stm gene of the present invention or a fragment or variant thereof may have at least 8 contiguous nucleotides in length. A nucleic acid molecule of the present invention has an appropriate nucleotide length which varies depending on the purpose of the present invention. More preferably, a nucleic acid molecule of the present invention may have at least 10 contiguous nucleotides in length, preferably at least 15 contiguous nucleotides in length, and more preferably at least 20 contiguous nucleotides in length. The lower limit of these nucleotide lengths may include values specifically described herein and values therebetween (e.g., 9, 11, 12, 13, 14, 16, etc.) or values more than those values (e.g., 21, 22, . . . , 30, etc.). The length of a nucleic acid molecule of the present invention may have an upper limit which is the full length of a sequence set forth in SEQ ID NO. 1, 3, 5 or 29 or more than the full length as long as it can be used in an application of interest (e.g., interaction with an antisense, RNAi, a marker, a primer, a probe, or a predetermined agent). Alternatively, when used as a primer, a nucleic acid molecule of the present invention may typically have at least about 8 nucleotides in length, and preferably about 10 nucleotides in length. When used as a probe, a nucleic acid molecule of the present invention may typically have at least about 15 nucleotides in length, and preferably about 17 nucleotides in length.

In another preferred embodiment, a nucleic acid molecule of the present invention has a sequence different from a sequence set forth in SEQ ID NO. 7 or 9 or a corresponding sequence in a corresponding nucleic acid sequence of Stm2 in at least one position in SEQ ID NO. 1, 3, 5 or 29. Such a position can be easily determined based on the alignment of at least 2 sequences of interest and the expression of the gene. Such a sequence may be specific only to the Stm1 gene, and therefore, is useful in distinguishing Stm1 from Stm2. Alternatively, the Stm1 gene and the Stm2 gene are definitely distinguished from each other in the presence or absence of the expression. Therefore, Stm1 and Stm2 can be distinguished from each other by observing the expression within cells. In a preferred embodiment, the portion having a different sequence may be digested with a restriction enzyme. Such a restriction enzyme can be easily determined by those skilled in the art if such a sequence is given. For example, in the present invention, when SEQ ID NOs. 3 and 9 are compared, at least 2 restriction enzymes, BsaMI which recognizes GAATGC and NlaIII which CATG, can be used.

(Stm Gene in Polypeptide Form)

In another aspect, the present invention relates to a product of the Stm gene (herein also referred to as a Stm gene product or a Stm polypeptide).

In a preferred embodiment, a polypeptide of the present invention comprises:

(a) a polypeptide consisting of an amino acid sequence set forth in SEQ ID NO. 2, 4, 6 or 30, or a fragment thereof;

(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;

(c) a polypeptide encoded by a spliced mutant or alleic mutant of a base sequence set forth in SEQ ID NO. 1, 3, 5 or 29;

(d) a polypeptide being a species homolog of an amino acid sequence set forth in SEQ ID NO. 2, 4, 6 or 30; or

(e) a polypeptide having at least 70% identity to any one of the polypeptides of (a) to (d) and having biological activity.

In one preferred embodiment, the number of substitutions, additions, and deletions in (b) is preferably limited to, for example, 50 or less, 40 or less, 30 or less, 20 or less, 15 or less, 10 or less, 9 or less, 8 or less, 7 or less, 6 or less, 5 or less, 4 or less, 3 or less, or 2 or less. The lesser number of substitutions, additions, and deletions is more preferable. However, such a number may be great as long as the Stm gene holds biological activity (preferably, the product of the Stm gene product is similar to the product of the Stm1 gene or the Stm gene has substantially the same activity as that of the Stm1 gene).

In another preferred embodiment, the alleic mutant of (c) preferably has at least about 90% homology to an amino acid sequence set forth in SEQ ID NO. 2, 4, 6 or 30. Preferably, the alleic mutant of (c) has at least about 99% homology to an amino acid sequence set forth in SEQ ID NO. 2, 4, 6 or 30.

In another preferred embodiment, the species homolog can be identified as described above. The species homolog preferably has at least about 30% homology to an amino acid sequence set forth in SEQ ID NO. 2, 4, 6 or 30. The species homology preferably has at least about 50% homology to a nucleic acid sequence set forth in SEQ ID NO. 1, 3, 5 or 29.

In another preferred embodiment, the biological activity of the variant polypeptide in (e) includes, for example, interaction with antibodies specific to a polypeptide consisting of an amino acid sequence set forth in SEQ ID NO. 2, 4, 6 or 30 or a fragment thereof, maintenance of an undifferentiated state, and the like. The present invention is not limited to this. Preferably, such biological activity includes maintenance of an undifferentiated state. Stm is considered to play an important role in the maintenance of an undifferentiated state of cells. Specifically, since the Stm gene has a homeodomain, it is inferred that the Stm gene suppresses expression of a downstream gene which, for example, induces differentiation of a tissue cell. It is considered that such activity can be measured by gene deletion experiments, RNAi experiments, experiments of inhibiting the function of a protein using antibodies, or the like.

In a preferred embodiment, the identity to any one of the polypeptides of (a) to (d) may be at least about 80%, more preferably at least about 90%, even more preferably at least about 98%, and most preferably at least about 99%.

A polypeptide of the present invention typically has at least 3 contiguous amino acid sequences. A polypeptide of the present invention has an amino acid length which may be any short length, and preferably, a longer length. Therefore, the amino acid length of a polypeptide of the present invention is preferably at least 4 amino acids in length, more preferably at least 5 amino acids in length, at least 6 amino acids in length, at least 7 amino acids in length, at least 8 amino acids in length, at least 9 amino acids in length, and at least 10 amino acids in length, even more preferably at least 15 amino acids in length, and still even more preferably at least 20 amino acids in length. The lower limit of these amino acid lengths may include values specifically described herein and values therebetween (e.g., 9, 11, 12, 13, 14, 16, etc.) or values more than those values (e.g., 21, 22, . . . , 30, etc.). The length of a polypeptide of the present invention may have an upper limit which is the full length of a sequence set forth in SEQ ID NO. 2, 4, 6 or 30 or more than the full length as long as it can be used in an application of interest (e.g., an immunogen, a marker, etc.).

In a preferred embodiment, a polypeptide of the present invention comprises:

(a) a polypeptide consisting of an amino acid sequence of positions 157 to 218 (homeodomain), positions 261 to 301 (W-rich region), or positions 399 to 455 (B2 repeat sequence region) set forth in SEQ ID NO. 4 or an amino acid sequence in corresponding positions, or a fragment thereof;

(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;

(c) a polypeptide having at least 70% identity to any one of the polypeptides of (a) to (b) and having biological activity.

A characteristic domain of the polypeptide, which can be used as such a Stm marker, includes regions encoded by the following positions of cDNA (SEQ ID NO. 3) corresponding to the polypeptide):

| Characteristic | |
|---|---|
| domain | cDNA position |
| Homeodomain | cDNA position: 469 bp-654 bp |
| W-rich region | cDNA position: 781 bp-903 bp |
| B2 repeated | cDNA position: 1195 bp-1365 bp |
| sequence region | |
| Octamer-bound | cDNA position: 1789 bp-1796 bp |
| sequence | (ACTAGCAT) |

and positions corresponding to genes corresponding thereto (e.g., a gene encoded by SEQ ID NO. 1 or 5, or SEQ ID NO. 7 or 9). The above-described region includes a polypeptide consisting of an amino acid sequence corresponding to positions 157 to 218 (homeodomain), positions 261 to 301 (W-rich region) or positions 399 to 455 (B2 repeat sequence region) in an amino acid sequence set forth in SEQ ID NO. 4, or a fragment thereof, or a position corresponding to the polypeptide (e.g., a gene encoded by a sequence set forth in SEQ ID NO. 2 or 6, or SEQ ID NO. 8 or 10). The present invention is not limited to this.

In a more preferred embodiment, the identity to any one of the polypeptides of (a) to (b) may be at least about 80%, more preferably at least about 90%, even more preferably at least about 98%, and most preferably at least about 99%.

In another preferred embodiment, a polypeptide of the present invention has a sequence, which is different from a sequence corresponding to SEQ ID NO. 8 or 10 or an amino acid sequence of Stm2 corresponding thereto, in at least one position in SEQ ID NO. 2, 4, 6 or 30. Such a position can be easily determined by aligning at least 2 sequences of interest. Such a sequence may be specific only to the Stm1 polypeptide, and therefore, is useful when distinguishing Stm1 from Stm2 is required. In a preferred embodiment, such a portion having a different sequence may be digested with peptidase or protease. Such an enzyme can be determined by those skilled in the art based on sequence information using a method well known in the art.

(Agent for Stm Gene in Nucleic Acid Form)

In one aspect, the present invention provides a composition comprising an agent capable of interacting specifically with a nucleic acid molecule encoding a Stm gene. Therefore, the present invention provides an agent specific to a nucleic acid molecule encoding any Stm gene described herein, or a variant or fragment thereof. An effective amount of the composition for diagnosis, prophylaxis, treatment or prognosis can be determined by those skilled in the art using techniques well known in the art with reference to various parameters, such as the purpose of use, a target disease (type, severity, and the like), the patient's age, weight, sex, and case history, the form or type of the cell, and the like. In the present invention, it was revealed that the expression of the Stm1 gene corresponds to an undifferentiated state (particularly pluripotency, and more specifically totipotency). Therefore, the present invention can be efficiently used to identify such a state and property. Particularly, the present invention is considered to provide a higher level of affinity to pluripotency or totipotency and a higher detection rate thereof than that of conventional Oct3/4. Such an effect was not conventionally known. Therefore, the agent of the present invention provides a more excellent effect or a different characteristic effect than conventional techniques.

In a preferred embodiment, an agent of the present invention may be an agent selected from the group consisting of nucleic acid molecules, polypeptides, lipids, sugar chains, low molecular weight organic molecules, and composite molecules thereof. It may be understood that such an agent may be any agent which is bound specifically to a nucleic acid molecule of the present invention.

In a preferred embodiment, an agent of the present invention is a nucleic acid molecule. When an agent of the present invention is a nucleic acid molecule, such a nucleic acid molecule may have at least 8 contiguous nucleotides in length, and preferably may be bound specifically to a nucleic acid sequence of Stm (e.g., SEQ ID NO. 1, 3, 5 or 29). A nucleic acid molecule of the present invention may have an appropriate nucleotide length which varies depending on the purpose of the application. More preferably, a nucleic acid molecule of the present invention may have at least 10 contiguous nucleotides in length, preferably at least 15 contiguous nucleotides in length, and more preferably at least 20 contiguous nucleotides in length. The lower limit of these nucleotide lengths may include values specifically described herein and values therebetween (e.g., 9, 11, 12, 13, 14, 16, etc.) or values more than those values (e.g., 21, 22, . . . , 30, etc.). The length of a nucleic acid molecule of the present invention may have an upper limit which is the full length of a sequence set forth in SEQ ID NO. 1, 3, 5 or 29 or more than the full length as long as it can be used in an application of interest (e.g., an antisense, RNAi, a marker, a primer, a probe, or a predetermined agent). Alternatively, when used as a primer, a nucleic acid molecule of the present invention may typically have at least about 8 nucleotides in length, and preferably about 10 nucleotides in length. When used as a probe, a nucleic acid molecule of the present invention may typically have at least about 15 nucleotides in length, and preferably about 17 nucleotides in length.

Therefore, in one illustrative embodiment, an agent of the present invention may be a nucleic acid molecule having a sequence complementary to a nucleic acid sequence of a polynucleotide encoding a Stm gene or a sequence having at least 70% identity thereto.

In another illustrative embodiment, an agent of the present invention may be a nucleic acid molecule hybridizable to a nucleic acid sequence of any one of the following Stm genes under stringent conditions: (a) a polynucleotide having a base sequence set forth in SEQ ID NO. 1, 3, 5 or 29, or a complementary or fragment thereof; (b) a polynucleotide consisting of an amino acid sequence set forth in SEQ ID NO. 2, 4, 6 or 30, or a polypeptide or encoding a fragment thereof; (c) a polynucleotide encoding a variant polypeptide having an amino acid sequence set forth in SEQ ID NO. 2, 4, 6 or 30, wherein at least one amino acid in the sequence has a mutation selected from the group consisting of substitution, addition, and deletion, or a fragment thereof, wherein the variant polypeptide has biological activity; (d) a polynucleotide hybridizable to any one of the polynucleotides (a) to (c) under stringent conditions and encoding a polypeptide having biological activity; or (e) a polynucleotide consisting of a base sequence having at least 70% identity to any one of the polynucleotides (a) to (c) or a complementary sequence thereof, and encoding a polypeptide having biological activity. Stringency may be high, moderate, or low, which can be determined by those skilled in the art as appropriate.

Alternatively, an agent of the present invention is preferably an agent specific to a nucleic acid molecule comprising a sequence set forth in SEQ ID NO. 1, 3, 5 or 29 or a complementary sequence thereof. Such a sequence can be used to identify the expression of the Stm1 gene present in a tissue which is in an undifferentiated state or has pluripotency (particularly totipotency). Therefore, such a sequence is useful in investigating the level of an undifferentiated state of a certain tissue or individual.

(An Agent for Stm Gene in Polypeptide Form)

In another aspect, the present invention relates to an agent capable of binding specifically to a polypeptide of the present invention and a composition comprising the same. Examples of such an agent include, but are not limited to, polypeptides (e.g., antibodies, single chain antibodies, etc.), polynucleotides, sugar chains, lipids, and composite molecules thereof, and the like. It may be understood that such an agent may be any one capable of binding specifically to a polypeptide of the present invention. More preferably, an agent of the present invention is an antibody or a derivative thereof (e.g., a single chain antibody, etc.). Therefore, an agent of the present invention can be used as a probe and/or an inhibitor. In a preferred embodiment, an agent of the present invention may be advantageously labeled or may be capable of binding to a label. When labeled, an agent of the present invention can be used to determine various conditions directly and/or easily. Such a label may be any one which can be distinguishably labeled, including, for example, but being not limited to, fluorescence, phosphorescence, chemiluminescence, radiation, enzyme-substrate reaction, antigen-antibody reaction, and the like. Alternatively, when the agent interacts with an antibody or the like via an immunological reaction, a biotin-streptavidin system, which is often used for immunological reactions, may be used.

In a preferred embodiment, an agent of the present invention may be an antibody. Such an antibody may be, for example, a monoclonal antibody, a polyclonal antibody, a humanized antibody thereof, a chimeric antibody, and an anti-idiotype antibody, and a fragment thereof (e.g., F(ab′)2 and Fab fragments, etc.), and other conjugates recombinantly produced. Such an antibody can be used as a tool for determination of expression of a gene of the present invention, and therefore, can be used for screening.

(Nucleic Acid Molecules of the Present Invention in Gene Engineered Form)

In another aspect, the present invention relates to an expression cassette and a vector comprising a nucleic acid molecule of the present invention. An expression cassette and a vector of the present invention preferably comprise a control sequence, which is operably linked to a nucleic acid molecule of the present invention. By comprising a control sequence, it becomes easy to control the expression of a nucleic acid molecule of the present invention. Examples of such a control sequence include, but are not limited to, a promoter sequence, an enhancer sequence, a terminator sequence, an intron sequence, and the like. Preferably, such a control sequence can induce the expression of a nucleic acid molecule of the present invention.

In a more preferred embodiment, an expression cassette and a vector of the present invention may further comprise a sequence encoding a selectable marker. Examples of such a selectable marker include, but are not limited to, an exogenous gene, a cellular gene, an antibiotic-resistant gene, and the like. Examples of an antibiotic-resistant gene include, but are not limited to, a neomycin-resistant gene, a hygromycin-resistant gene, and the like. Examples of a cellular gene include, but are not limited to, a gene encoding a cytokine (e.g., a growth factor, etc.), a gene encoding a growth factor receptor, a gene encoding signal transduction molecule, a gene encoding a transcription factor, and the like. In another preferred embodiment, a selectable marker may be an immortalizing gene (e.g., bcl-2, etc.). Alternatively, a selectable marker may be hypoxanthine phosphoribosyl transferase (HPRT), a gene encoding a toxic product, a toxic gene product combined with a suicide substrate which is active depending on conditions (e.g., herpes simplex virus thymidine kinase (HSV-TK) combined with ganciclovir, etc.), and a herpes simplex virus thymidine kinase (HSV-TK) gene.

(Cell Form)

In another aspect, the present invention relates to a cell comprising a nucleic acid sequence encoding a Stm gene (e.g., a nucleic acid molecule of the present invention, etc.). A method for introducing a nucleic acid molecule of the present invention into cells is well known in the art, and is described above in detail. Alternatively, such a cell can be identified by screening cells contained in a sample for a cell having such a nucleic acid molecule. A cell containing a nucleic acid molecule of the present invention may be preferably in an undifferentiated state. A cell in which a nucleic acid molecule of the present invention is expressed is typically in an undifferentiated state. Therefore, it is possible to control the undifferentiated state of a cell into which such a nucleic acid molecule has been introduced in a manner which allows the molecule to be controllably expressed. Alternatively, it is possible to use such a cell to produce a large amount of a nucleic acid molecule of the present invention. Such a production method is well known in the art, and is described in the documents mentioned herein.

(Tissue Form)

In another aspect, the present invention relates to a tissue comprising a nucleic acid sequence encoding a Stm gene. Preferably, such a nucleic acid sequence is operably linked to a control sequence. Such a tissue may be an animal tissue or a tissue derived from other organisms (e.g., plants, etc.). Alternatively, such a tissue can be used to produce a large amount of a nucleic acid molecule of the present invention. Such a production method is well known in the art, and is described in the documents mentioned herein.

(Organism Form)

In another aspect, the present invention relates to an organism (e.g., an animal, etc.) comprising a nucleic acid sequence encoding a Stm gene. Preferably, such a nucleic acid sequence is operably linked to a control sequence. Such an organism may be an animal or other organisms (e.g., plants, etc.). Alternatively, such an animal can be used to produce a large amount of a nucleic acid molecule of the present invention. Such a production method is well known in the art, and is described in the documents mentioned herein. If a Stm gene suppresses the expression of a gene specific to differentiated cells, there is a possibility that induction of differentiation in a certain direction can be suppressed. In other words, such a Stm gene has a function to determine the direction of differentiation and is considered to be applicable to regenerative medicine.

(Concentrated Composition Form)

In another aspect, the present invention relates to a composition, in which cells containing a nucleic acid molecule of the present invention (e.g., a nucleic acid molecule encoding the Stm1 gene, etc.) are concentrated. Such a cell is typically in an undifferentiated state when a Stm gene is expressed. A composition having the concentrated cells can be said to contain a greater number of undifferentiated cells (e.g., a pluripotent stem cell, an embryonic stem cell, etc.) than conventional compositions. A method for concentrating cells containing such a nucleic acid molecule is well known in the art, including, for example, a method using immunophenotyping (e.g., magnetic separation using magnetic beads coated with antibodies, panning, flow cytometry, etc.). The present invention is not limited to this.

In another aspect, the present invention relates to a nucleic acid molecule comprising a sequence of a promoter portion of a Stm gene. Such a promoter portion may be a region (200 bp in length) between 1300 bp to 1500 bp upstream of a transcription start site (ATG) of a sequence set forth in SEQ ID NO. 1, 3, 5 or 29. This region has a sequence to which Sp1 and AP-2 can bind and is expected to play an important role in controlling gene expression. In the case of a Stm gene, expression is observed in transgenes including 2.5 kb 5′ upstream of a transcription start site and 3.9 kb 3′ downstream of a poly-A sequence in the genomic base sequence of Stm1. Therefore, it is inferred that a Stm gene contains a base sequence (promoter region) necessary and sufficient for transcription at least in these regions. The accurate positions of these regions can be easily determined by those skilled in the art using well-known and commonly used techniques. Therefore, accurate positions identified by such a technique are also encompassed by the present invention.

(Promoter Form)

In another aspect, the present invention relates to a vector comprising a nucleic acid sequence of a promoter portion of a Stm gene (herein referred to as a Stm gene promoter). In a vector of the present invention, preferably, an exogenous gene (e.g., a marker gene, etc.) is operably linked to a nucleic acid sequence of a promoter portion of a Stm gene. By transforming cells with a vector having such a structure, an undifferentiated state of cells can be observed as the expression of the above-described exogenous gene. Therefore, a gene encoding a fluorescent material (e.g., a green fluorescence gene, etc.) can be used as a marker gene to observe cells in an undifferentiated state. Such an exogenous gene is preferably non-toxic to the cell. More preferably, when implantation is intended, such an exogenous gene may be advantageously non-toxic to a host for implantation.

In a more preferred embodiment, a vector comprising a Stm gene promoter of the present invention may further comprise a sequence encoding a selectable marker. Examples of such a selectable marker include, but are not limited to, an exogenous gene, a cellular gene, an antibiotic-resistant gene, and the like. Examples of an antibiotic-resistant gene include, but are not limited to, a neomycin-resistant gene, a hygromycin-resistant gene, and the like. Examples of a cellular gene include, but are not limited to, a gene encoding a cytokine (e.g., a growth factor, etc.), a gene encoding a growth factor receptor, a gene encoding signal transduction molecule, a gene encoding a transcription factor, and the like. In another preferred embodiment, a selectable marker may be an immortalizing gene (e.g., bcl-2, etc.). Alternatively, a selectable marker may be HPRT, a gene encoding a toxic product, a toxic gene product combined with a suicide substrate which is active depending on conditions, and a herpes simplex virus thymidine kinase (HSV-TK) gene.

(Cells Having a Promoter)

In another aspect, the present invention relates to a cell containing a Stm gene promoter. A method for introducing a nucleic acid sequence encoding a Stm gene promoter of the present invention into cells is well known in the art, and is described in detail herein above. Alternatively, such a cell can be identified by screening cells contained in a sample for a cell containing a Stm gene promoter. When an exogenous gene (e.g., a marker gene, etc.) is operably linked to a nucleic acid sequence of a promoter portion of a Stm gene, the undifferentiated state of cells can be determined by observing the expression of the exogenous gene.

(Tissue Having a Promoter)

In another aspect, the present invention relates to a tissue containing a nucleic acid sequence encoding a Stm gene promoter. Preferably, such a nucleic acid molecule is operably linked to an exogenous gene (e.g., a marker gene, etc.). Such a tissue may be an animal tissue or a tissue of other organisms (e.g., plants, etc.).

(Organisms Having a Promoter)

In another aspect, the present invention relates to an organism (e.g., an animal, etc.) comprising a nucleic acid sequence encoding a Stm gene. Preferably, such a nucleic acid sequence is operably linked to a control sequence. Such an organism may be an animal or other organisms (e.g., plants, etc.). If a Stm gene suppresses the expression of a gene specific to differentiated cells, there is a possibility that induction of differentiation in a certain direction can be suppressed. In other words, such a Stm gene has a function to determine the direction of differentiation and is considered to be applicable to regenerative medicine.

(Composition Containing Concentrated Cell Having a Promoter)

In another aspect, the present invention relates to a composition, in which cells containing a nucleic acid molecule encoding the Stm1 gene are concentrated. Such a cell is typically in an undifferentiated state when a gene whose expression is induced by a Stm promoter is expressed. A composition having such concentrated cells can be said to contain a greater number of undifferentiated cells (e.g., a pluripotent stem cell, an embryonic stem cell, etc.) than conventional compositions. Methods for concentrating cells containing such a nucleic acid molecule are well known in the art, including, for example, methods using immunophenotyping (e.g., magnetic separation using magnetic beads coated with antibodies, panning, flow cytometry, etc.). The present invention is not limited to this.

(Undifferentiated State Determination Composition)

In another aspect, the present invention relates to a composition for determining an undifferentiated state of cells. The composition comprises an agent which reacts specifically with a Stm gene or a Stm gene product. Such an agent includes, but is not limited to, an agent which interacts specifically with a Stm gene (e.g., a nucleic acid molecule having a complementary sequence, a polypeptide (e.g., a transcription agent, etc.)), an antibody for a Stm gene product, a single chain antibody, and the like. A Stm gene or a Stm gene product used herein may be a nucleic acid molecule or a polypeptide having a sequence as described herein above. The present invention is not limited to this. Those skilled in the art can alter such a nucleic acid molecule and polypeptide using techniques well known in the art. Such alterations can be modified as appropriate by those skilled in the art depending on the purpose of the application.

A subject to be determined by a composition for determining an undifferentiated state of cells of the present invention preferably includes stem cells. The Stm gene of the present invention was revealed to be expressed in stem cells (e.g., neural stem cells, etc.) more universally than conventional agents (e.g., Oct3/4, etc.). In addition, the Stm gene of present invention is not expressed in cells other than stem cells (e.g., unfertilized egg cells, etc.). Such a property is not achieved by conventional agents, such as Oct3/4 and the like. Therefore, such a composition of the present invention can advantageously determine the presence or absence of stem cells more universally than systems using conventional agents. Such an advantage is a significant effect which is difficult for conventional agents (e.g., Oct3/4, etc.) to achieve. In addition, it can be said that the control of expression of downstream genes can be determined with more accuracy if the relationship between Oct3/4 and Stm is taken into account.

In a preferred embodiment, a stem cell may be selected from the group consisting of an embryonic stem cell, a pluripotent stem cell, a unipotent stem cell, and a tissue stem cell. A composition of the present invention has a novel advantage of being used for determination of general pluripotent stem cells, particularly including tissue stem cells. This is because conventional markers cannot distinguish totipotent stem cells (e.g., embryonic stem cells, fertilized egg cells, etc.) from tissue stem cells which are differentiated to some degree. Examples of a stem cell intended herein include, but are not limited to, fertilized egg cells, embryonic stem cells, neural stem cells, retinal stem cells, follicular stem cells, pancreatic (common) stem cells, hepatic stem cells, hematopoietic stem cells, mesenchymal stem cells, gonadal stem cells, epidermic stem cells, mesenchymal tissue stem cells, embryonic stem cells, embryonic germ cells, and the like. Preferably, such stem cells include, but are not limited to, neural stem cells, hematopoietic stem cells, epidermic stem cells, mesenchymal tissue stem cells, and the like. Although not wishing to be bound by theory, an effect of the present invention is that totipotent cells can be determined with more accuracy. Therefore, the present invention may be used to determine a reprogrammed state. Although not wishing to be bound by theory, it is considered that the Stm1 gene of the present invention is reactivated (expressed) at an earlier stage when a somatic cell is reprogrammed. For example, it is considered that a tissue stem cell Stm1(+)/Oct3/4(−) is reprogrammed into Stm1(+)/Oct3/4(+).

A cell targeted by a composition of the present invention may be either a genetically modified cell or a non-genetically modified cell (i.e., a naturally-occurring cell, etc.). Methods for genetic modification are well known in the art, and is described in detail in documents mentioned herein. Those skilled in the art can genetically modify cells as appropriate using such well known and commonly used techniques. Therefore, such a cell may be a differentiated cell which is genetically engineered to be in an undifferentiated state (i.e., pluripotency is imparted).

(Method for Determining Undifferentiated State)

In another aspect, the present invention provides a method for determining an undifferentiated state of cells. The method comprises the steps of: (I) providing a cell to be determined; (II) contacting an agent capable of reacting specifically with a Stm gene or a Stm gene product with the cell; and (III) determining whether or not the Stm gene is expressed in the cell. The cell provided may be any cell which is desired to be determined. Such a cell may be provided in any form, and preferably in a form appropriate for assay. For example, the cell may be provided in an appropriate medium or buffered solution. The present invention is not limited to this. The agent capable of reacting specifically with a Stm gene or a Stm gene product may be in any form as long as it can react with a Stm gene or a Stm gene product. Such a Stm gene or Stm gene product is preferably derived from the same species as that of a cell to be determined. If a Stm gene or a Stm gene product is derived from the same species as that of a cell to be determined, the presence or absence of the Stm gene or Stm gene product in the cell can be determined with substantially one-to-one correspondence. Note that the species from which the above-described agent is derived may be different from that of a Stm gene or a Stm gene product to be determined as long as the Stm gene or the Stm gene product can be determined. This is because cross reactions often occur between different species. The above-described Stm gene or Stm gene product may be, but is not limited to, a nucleic acid molecule or polypeptide as described herein. Those skilled in the art can alter such a nucleic acid molecule and polypeptide using techniques well known in the art. Such alterations can be modified as appropriate by those skilled in the art depending on the purpose of the application.

A method for determining an undifferentiated state of cells of the present invention preferably further comprises determining whether or not other stem cell markers are expressed. By determining expression of other stem cell markers, an undifferentiated state can be determined with higher accuracy. Examples of such other stem cell markers include, but are not limited to, Oct3/4, UTF1, Sox1, Rex1, and the like. A Stm gene used in the present invention preferably includes the Stm1 gene. This is because Stm1 has been demonstrated to be definitely associated with stem cells.

(Method for Preparing an Undifferentiated Cell)

In another aspect, the present invention relates to a method for preparing cells in an undifferentiated state. The preparation method comprises the steps of: (I) providing a sample known or suspected of containing cells in an undifferentiated state; (II) contacting an agent capable of reacting specifically with a Stm gene or a Stm gene product with the sample; (III) detecting a specific reaction between the agent and the Stm gene or Stm gene product to determine whether or not the Stm gene is expressed in cells of the sample; and (IV) isolating or concentrating the cells in which the Stm gene is expressed. The above-described sample may be any one which is known or suspected of containing cells in an undifferentiated state. A cell used herein may be any cell, preferably including cells derived from mammalian animals (e.g., monotremata, marsupialia, edentate, dermoptera, chiroptera, carnivore, insectivore, proboscidea, perissodactyla, artiodactyla, tubulidentata, pholidota, sirenia, cetacean, primates, rodentia, lagomorpha, etc.), and more preferably a cell derived from human. The step of preparing a sample can be performed by using techniques well known in the art. Such techniques are described in detail in the documents mentioned herein. For example, cells are removed from an animal, and thereafter, the cells are placed in an appropriate medium or buffered solution or the like. The present invention is not limited to this. The step of contacting an agent of the present invention with a sample can be performed by using techniques well known in the art. Examples of such a technique includes, but is not limited to, adding a solution containing an agent of the present invention into a sample. The step of detecting a specific reaction between an agent of the present invention and a Stm gene or a Stm gene product can be performed by using techniques well known in the art. For convenience of detection, the agent is preferably labeled. Such label may be any label, including, but being not limited to, a fluorescent label, a chemiluminescent label, a radiolabel, and the like. Alternatively, when the agent interacts with an antibody or the like via an immunological reaction, a system often used for an immunological reaction, such as a biotin-streptavidin system or the like, may be used. Gene expression can be correlated with the presence or absence of such a specific reaction. For example, the known expression level of a cell is correlated with the strength of a specific reaction to prepare a standard curve. By utilizing such a standard curve, gene expression can be qualitatively or quantitatively determined from a specific reaction.

A Stm gene or a Stm gene product used in a method for preparing cells in an undifferentiated state may be, but is not limited to, a nucleic acid molecule or a polypeptide as described herein. Those skilled in the art can alter such a nucleic acid molecule and polypeptide using techniques well known in the art. Such alterations can be modified as appropriate by those skilled in the art depending on the purpose of the application.

(Method for Preparing Undifferentiated Cells)

In another aspect, the present invention relates to another method for preparing cells in an undifferentiated state. The method comprises the steps of: (I) providing cells; and (II) inducing expression of a Stm gene in the cell. A cell used herein may be any cell, preferably including cells derived from mammalian animals (e.g., monotremata, marsupialia, edentate, dermoptera, chiroptera, carnivore, insectivore, proboscidea, perissodactyla, artiodactyla, tubulidentata, pholidota, sirenia, cetacean, primates, rodentia, lagomorpha, etc.), and more preferably a cell derived from human. A step of introducing a gene into cells can be performed by a technique well known in the art. Any technique can be used as long as it can introduce a gene of interest (e.g., a Stm gene, etc.) into cells. Examples of such a technique include, but are not limited to, transfection, transduction, transformation, and the like (e.g., a calcium phosphate method, a liposome method, a DEAE dextran method, an electroporation method, a particle gun (gene gun) method, etc.). A Stm gene is preferably introduced with a vector into cells. Such a vector may be any vector, preferably including pGEM, pBluescript KS+/, and the like.

A Stm gene or a Stm gene product used in a method for preparing cells in an undifferentiated state, which is characterized by the step of inducing the expression of a Stm gene, may be, but is not limited to, a nucleic acid molecule or a polypeptide as described herein. Those skilled in the art can alter such a nucleic acid molecule and polypeptide using techniques well known in the art. Such alterations can be modified as appropriate by those skilled in the art depending on the purpose of the application.

(Method for Concentrating Undifferentiated Cells)

In another aspect, the present invention provides a method for isolating and/or growing and/or concentrating cells in an undifferentiated state. The method comprises the steps of: (I) providing cells; (II) introducing a Stm gene or a Stm gene promoter into the cell; and (III) selecting the cell in which the Stm gene or the Stm gene promoter is expressed. A cell used herein may be any cell, preferably including cells derived from mammalian animals (e.g., monotremata, marsupialia, edentate, dermoptera, chiroptera, carnivore, insectivore, proboscidea, perissodactyla, artiodactyla, tubulidentata, pholidota, sirenia, cetacean, primates, rodentia, lagomorpha, etc.), and more preferably a cell derived from human. A step of introducing a gene into cells can be performed by techniques well known in the art. Any technique can be used as long as it can introduce a gene of interest (e.g., a Stm gene, etc.) into cells. Examples of such a technique include, but are not limited to, transfection, transduction, transformation, and the like (e.g., a calcium phosphate method, a liposome method, a DEAE dextran method, an electroporation method, a particle gun (gene gun) method, etc.). A Stm gene is preferably introduced with a vector into cells. Such a vector may be any vector, preferably including pGEM, pBluescript KS+/, and the like.

A Stm gene or Stm gene product used in the method of the present invention for isolating and/or growing and/or concentrating cells in an undifferentiated state, may be, but is not limited to, a nucleic acid molecule or a polypeptide as described herein. Those skilled in the art can alter such a nucleic acid molecule and polypeptide using techniques well known in the art. Such alterations can be modified as appropriate by those skilled in the art depending on the purpose of the application.

(Kit for Determining an Undifferentiated State)

In another aspect, the present invention provides a kit for determining a differentiated state of a cell. The kit comprises (a) an agent capable of reacting specifically with a Stm gene or a Stm gene product; and (b) means for determining whether or not the Stm gene is expressed in the cell. Any agent as described herein can be used as an agent capable of reacting specifically with a Stm gene or a Stm gene product. Examples of such an agent include, but are not limited to, an antibody, a nucleic acid molecule, and the like. Therefore, examples of such an agent include, but are not limited to, an agent capable of interacting specifically with a Stm gene or a Stm gene product (e.g., a nucleic acid molecule having a complementary sequence, a polypeptide such as a transcription agent or the like, etc.), an antibody or a single chain antibody against a Stm gene product, and the like. Means for determining gene expression can be performed using techniques well known in the art. Examples of such determination means include, but are not limited to, dot blot analysis, Northern blot analysis and the like (analysis on mRNA as a gene product), Western blot analysis, ELISA, and the like (analysis on a polypeptide as a gene product), and the like. For such analysis, for example, a microtiter plate, a microarray, and the like can be used.

A Stm gene or a Stm gene product used in a kit for determining a differentiated state of a cell of the present invention may be a nucleic acid molecule or a polypeptide as described herein. Such a nucleic acid molecule and polypeptide can be modified by those skilled in the art using techniques well known in the art. Such alterations can be modified as appropriate by those skilled in the art depending on the purpose of the application.

In a preferred embodiment, a kit for determining a differentiated state of a cell of the present invention further comprises means for determining whether or not another stem cell marker is expressed. Such means for determining expression of another stem cell marker may be based on the same principle of means for determining expression of a Stm gene of the present invention or based on other different principles. Preferably, a result presented by such means for determining expression of another stem cell marker is preferably represented in a manner which distinguishes it from a result presented by means for determining expression of a Stm gene of the present invention (e.g., a different color, different fluorescence, etc.). Examples of such another stem cell marker include, but are not limited to, Oct3/4, UTF1, Sox1, Rex1, and the like.

In a preferred embodiment, a Stm gene used in a kit of the present invention includes a Stm1 gene.

(Kit for Preparing Undifferentiated Cell)

In another aspect, the present invention provides a kit for preparing a cell in an undifferentiated state. The kit comprises (I) an agent capable of reacting specifically with a Stm gene or a Stm gene product; (II) means for determining whether or not the Stm gene is expressed in a cell in a sample; and (III) means for isolating or concentrating a cell in which the Stm gene is expressed.

An agent capable of reacting specifically with a Stm gene or a Stm gene product used in a kit for preparing a cell in an undifferentiated state of the present invention may be, in principle, the same as that used in a kit for determining a differentiated state of a cell of the present invention. Preferably, an agent appropriate for isolation or concentration of a cell may be used. For example, a cell sorter may be used in a cell sorting kit using anti-Stm1 antibodies or purification may be performed using beads having attached anti-Stm1 antibodies.

Means for determining whether or not a Stm gene is expressed in a cell in a sample may be, in principle, the same as that used in a kit for determining a differentiated state of a cell of the present invention. Preferably, a kit for preparing a cell in an undifferentiated state may further comprise means for determining whether or not another stem cell marker is expressed. Such means for determining whether or not another stem cell marker is expressed may be, in principle, the same as that used in a kit for determining a differentiated state of a cell of the present invention.

Any means for isolating or concentrating a cell, which is used in the art, can be used as means for isolating or concentrating a cell, in which a Stm gene is expressed, and used in a kit for preparing a cell in an undifferentiated state of the present invention. Examples of such isolation or concentration means include, but are not limited to, magnetic separation, panning, flow cytometry, FACS, affinity chromatography, and the like.

(Kit for Preparing Undifferentiated Cell)

In another aspect, the present invention provides a kit for preparing a cell in an undifferentiated state. The kit comprises (I) means for inducing expression of a Stm gene in a cell. Such means for inducing expression of a Stm gene may be a technique well known in the art. As described herein, an antibody and cell sorting, which use a polypeptide of the present invention, can be used in accordance with the description of the present specification. Therefore, in this case, they are provided in the form of a kit, or in the form of sale of an antibody or a kit of an antibody with optimal buffer. Surface antigens can be purified using beads having attached antibodies, though Stm1 is localized in nuclei. If such a point is taken into consideration, the above-described kit can be easily implemented. A Stm gene used herein may be a nucleic acid molecule or a polypeptide as described herein. The present invention is not limited to this. Those skilled in the art can modify such a nucleic acid molecule and polypeptide using techniques well known in the art. Such alterations can be modified as appropriate by those skilled in the art depending on the purpose of the application.

In another aspect, a kit for preparing a cell in an undifferentiated state of the present invention comprises (I) a vector containing a Stm gene operably linked to a control sequence. Such a control sequence may be a promoter, an enhancer, a terminator, or the like well known in the art. A Stm gene contained in a vector may be, but is not limited to, a nucleic acid molecule or polypeptide as described herein. Those skilled in the art can modify such a nucleic acid molecule and polypeptide using techniques well known in the art. Such alterations can be modified as appropriate by those skilled in the art depending on the purpose of the application.

All scientific publications, patents, patent applications and the like cited herein are incorporated by reference in their entireties as if set forth fully herein.

The present invention has heretofore been described by way of preferred embodiments for a better understanding of the present invention. Hereinafter, the present invention will be described by way of examples. Examples described below are provided only for illustrative purposes and are not intended to limit the present invention. Accordingly, the scope of the present invention is not limited by embodiments and examples specified herein except as by the appended claims.

### EXAMPLES

In the examples below, animals were cared in accordance with rules defined by Kyoto University (Japan).

### Example 1

### Recovery of RNA

In Example 1, a Stm gene was identified.

Total RNA was recovered from tissues using Trizol reagent (GIBCO-BRL) in accordance with manufacture's instructions. The tissues were the brain, thymus, lung, heart, liver, kidney, spleen, testis, ovary, and muscle of 8 week old adult mice, and E6.5, E7.5, E8.5, E9.5, E12.5, and E18.5-day-old mouse fetuses. In addition, RNA was recovered from the genital ridge, unfertilized eggs, morula, blastocyst, embryonic stem cells and EG cells derived from E12.5-day-old male and female mouse fetuses for experiments.

### Example 2

### Northern Blot Hybridization Analysis

A general protocol (Alwine et. al., 1977, Proc. Natl. Acad. Sci., 74: 5350) was used to perform Northern blot hybridization analysis. Total RNA (10 μg), which had been extracted from embryonic stem cells, EG cells, and E12.5-day-old mouse fetuses, was dissolved in water, followed by electrophoresis using 1% formaldehyde degeneration gel. Thereafter, Hybond-N+ membrane (Amersham Biosciences) was used to perform blotting overnight. The blotted membrane was subjected to prehybridization at 42° C. for 2 hours and then hybridization using a specific probe overnight. Thereafter, the membrane was washed twice with 2×SSC/0.1% SDS at 65° C., and once with 0.1×SSC/0.1% SDS. The probe was labeled with [α-32P] dCTP (Amersham Biosciences) RI label using Megaprimer DNA labeling system (Amersham Biosciences) with respect to the full length of Stm1 cDNA.

### Example 3

### Gene Expression Analysis by RT-PCR

For the purpose of expression analysis of Stm1, Oct3/4, and G3pdh genes by RT-PCR, an Oligo-dT primer was used to perform cDNA synthesis. RNA samples were treated with DNaseI. Thereafter, RT reaction was performed using Superscript II RT (GIBCO BRL) in accordance with manufacture's instructions. PCR amplification was performed using 1 μg of total RNA. A set of primers used are described below:

F1:

(SEQ ID NO. 11)

5′-GCGCATTTTAGCACCCCACA-3′
and
R1:

(SEQ ID NO. 12)

5′-GTTCTAAGTCCTAGGTTTGC-3′;
F2:

(SEQ ID NO. 13)

5′-GAATTCTGGGAACGCCTCAT-3′
and
R2:

(SEQ ID NO. 14)

5′-CCAGATGTTGCGTAAGTCTC-3′;
Oct3/4RT/1:

(SEQ ID NO. 15)

5′-GGCGTTCTCTTTGGAAAGGTGTTC-3′
and
Oct-4RT/2:

(SEQ ID NO. 16)

5′-CTCGAACCACATCCTTCTCT-3′;
G3PDH-5:

(SEQ ID NO. 17)

5′-TGAAGGTCGGTGTCAACGGATTTGGC-3′
and
G3PDH-3:

(SEQ ID NO. 18)

5′-CATGTAGGCCATGAGGTCCACCAC-3′.

PCR was performed under the following conditions: 5-min incubation at 94° C.; 30 cycles of 94° C. for 30 seconds, 60° C. for 30 seconds, and 72° C. for 1 min; and finally 5-min incubation at 72° C.

### Example 4

### Expression of the Stm1 Gene within Cells

For the purpose of observing expression of the Stm1 gene within cells, a myc-Stm1 construct (myc-tagged Stm1 gene) was prepared. Stm1 cDNA was obtained by TA cloning of a product which had been obtained using the following primer set, using pGEM-T Easy vector system (Promega):

Stm-f:

(SEQ ID NO. 19)

5′-CGGGATCCATGAGTGTGGGTCTTCCTG G-3′
and
Stm-r:

(SEQ ID NO. 20)

5′-TCCCCCGGGTCATATTTCACCTGGTGGAG-3′.

The plasmid was cut with restriction enzymes BamHI and SmaI and blunt-ended. The blunt-ended cDNA fragment was cloned into a blunt-ended SalI site of pCMV-myc (CLONTECH), thereby producing pCMV-myc-Stm1 plasmid. pCMV-myc-Stm1 (1 μg) was introduced into 1×10⁵ embryonic stem cells using Lipofectamine 2000 Reagent (GIBCO-BRL).

### Example 5

### Immunological Cell Staining

Embryonic stem cells having pCMV-myc-Stm1 introduced therein were fixed with 4% PFA and immunologically stained using a standard immunological staining method for cultured cells (Willingham, M. C. et. al., 1985, An Atlas of Immunofluorescence in Cultured Cell, Academic Press, Orlando, Fla., pp. 1-13). Blocking was performed with 0.1% Triton X/PBS/2% skim milk at room temperature for 1 hour. Washing was performed four times with 0.1% Triton X/PBS at room temperature for 5 minutes. As a primary antibody, 1/100 dilution of 200 μg/mL c-myc monoclonal antibody (CLONTECH) was used. As a secondary antibody, 1/200 dilution of FITC label goat anti-mouse IgG (H+ L) (ZYMED LABORATORIES, INC) was used. After reaction using the secondary antibody, rhodamine phalloidin (Molecular Probes) and DAPI (SIGMA) were used in sequence for staining and signals were detected.

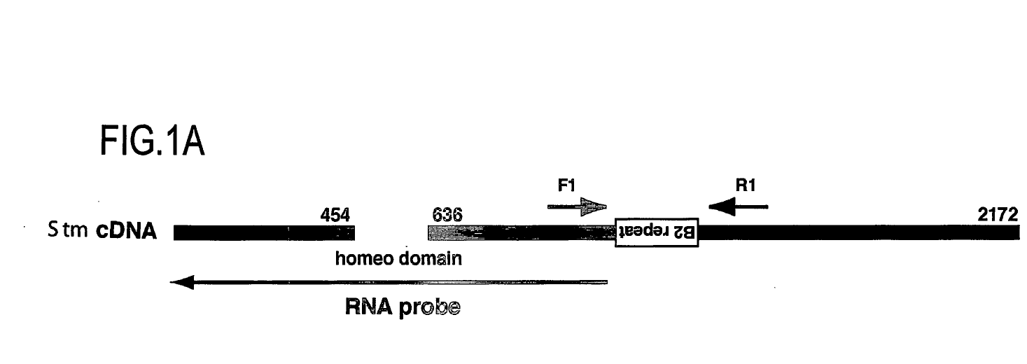

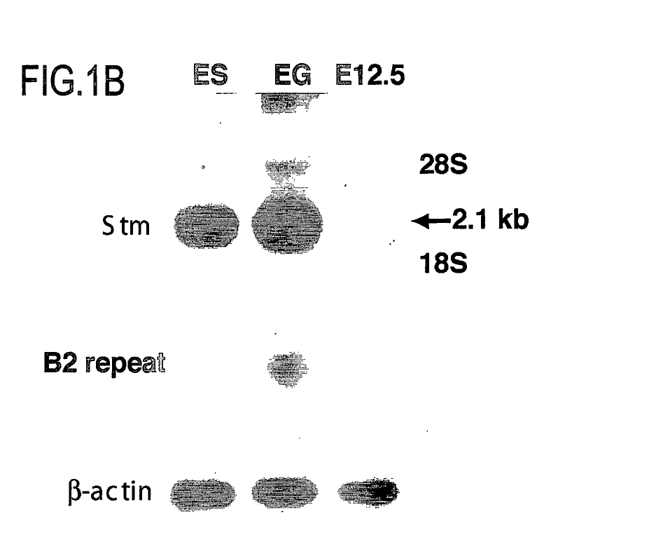

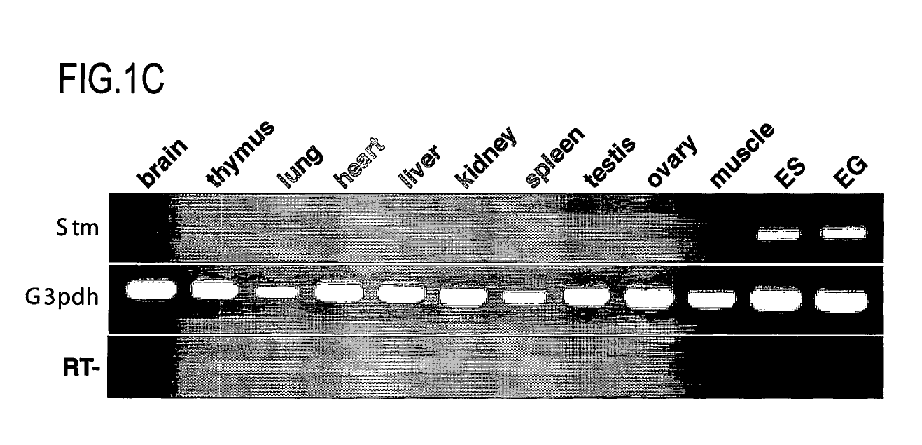

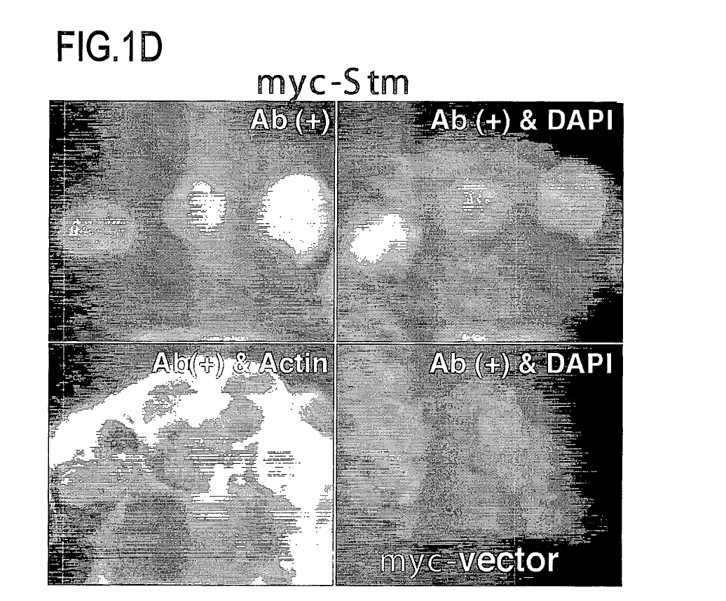

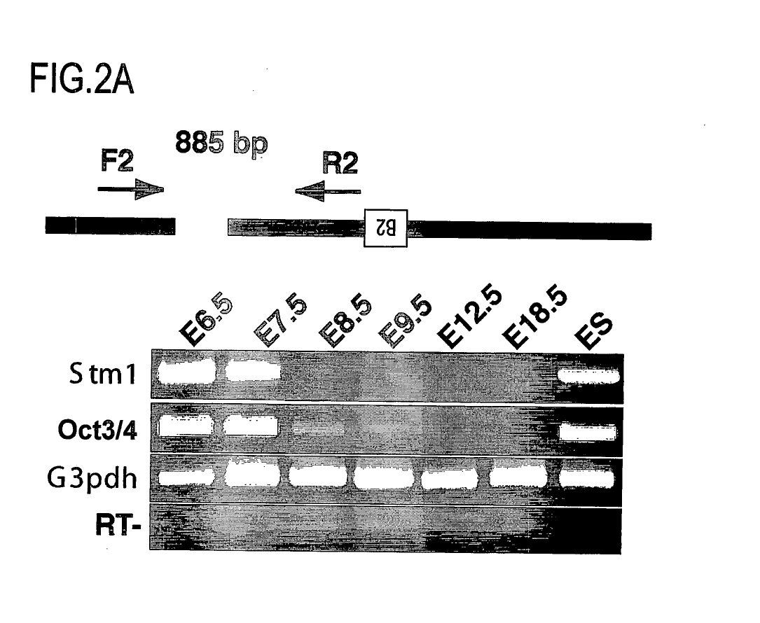

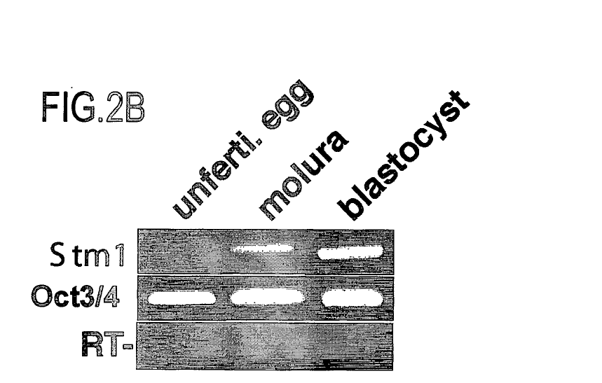

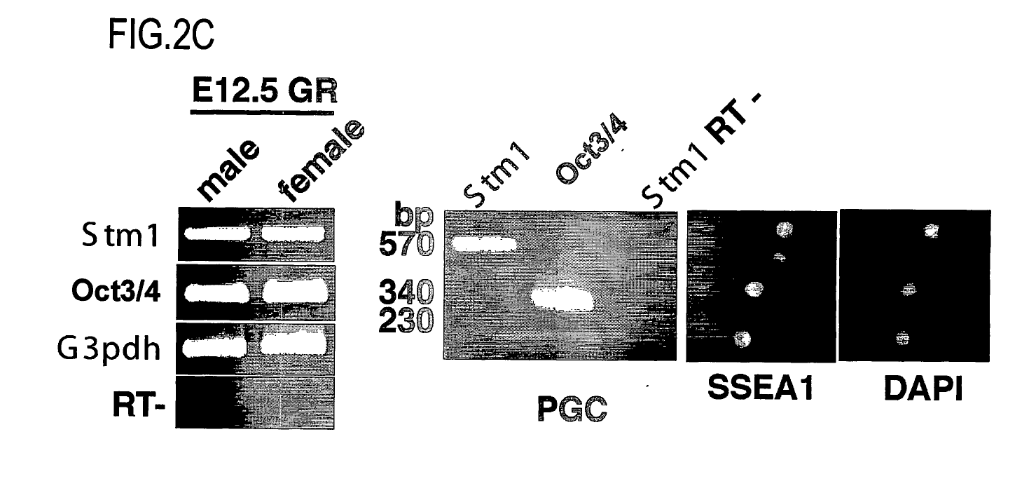

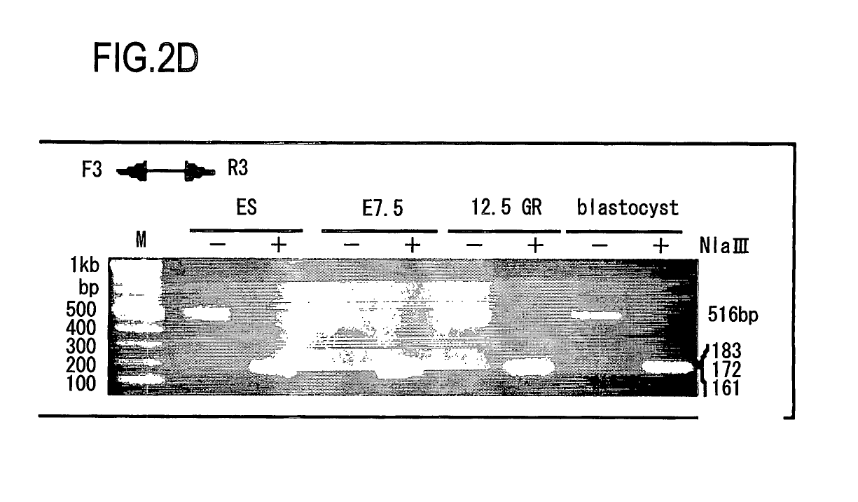

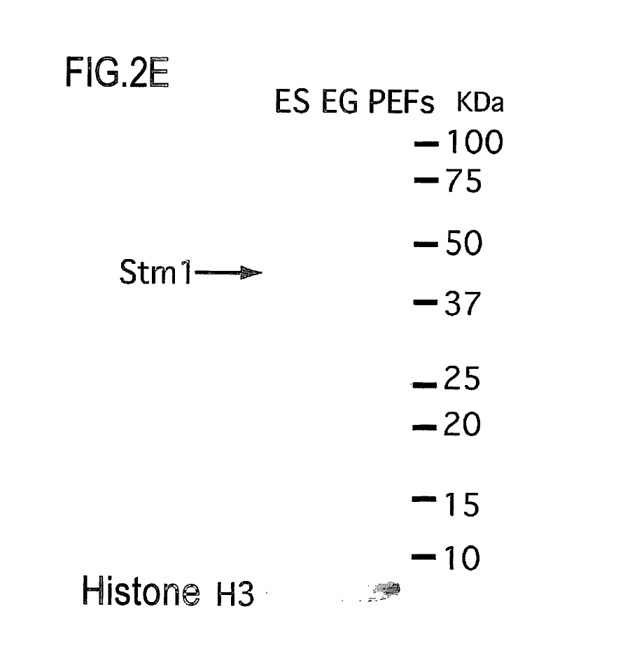

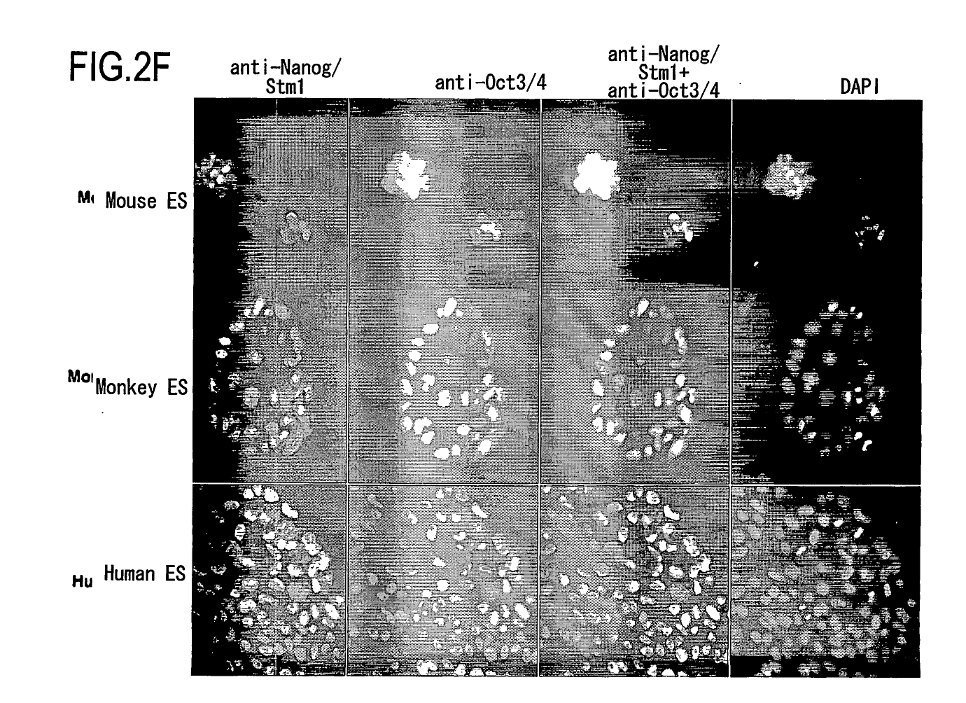

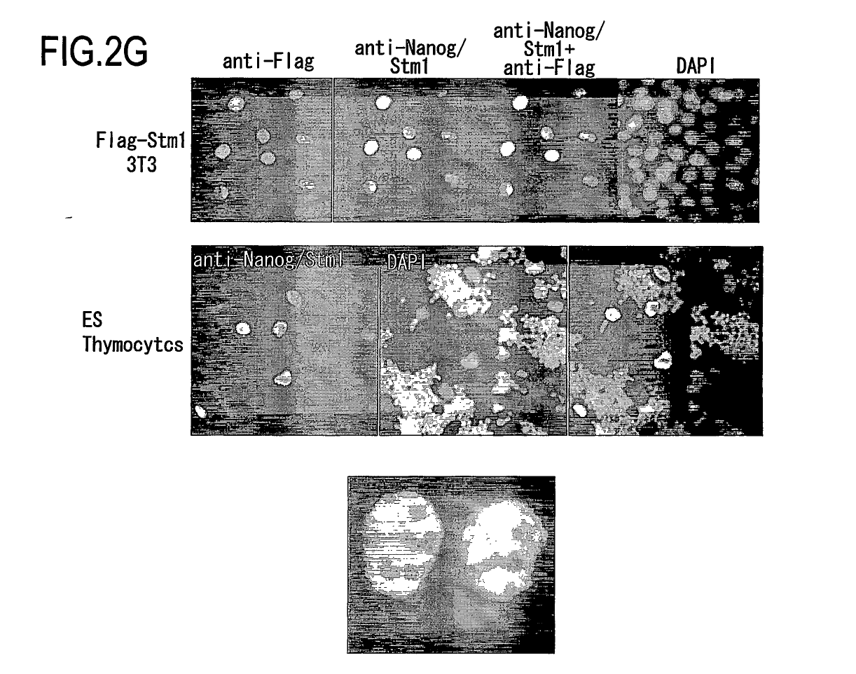

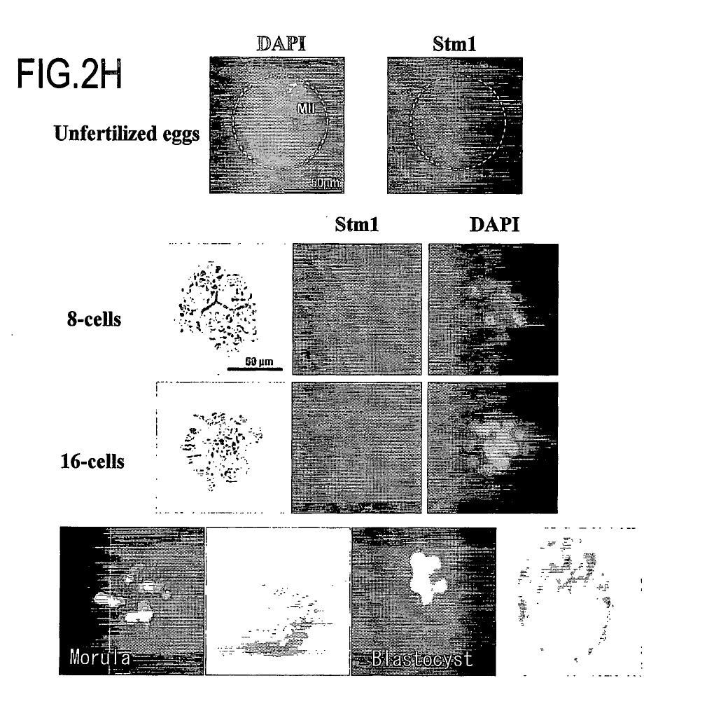

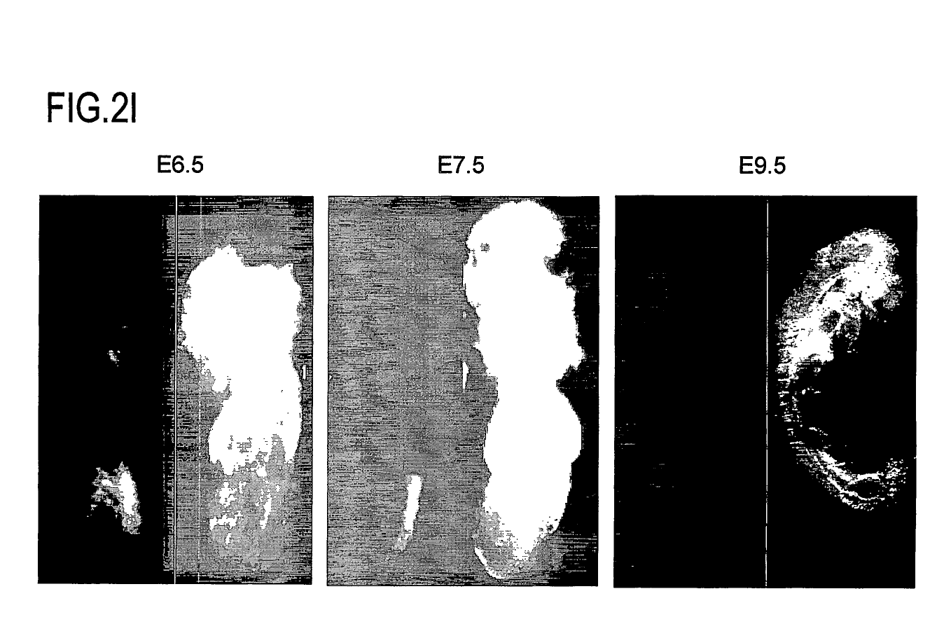

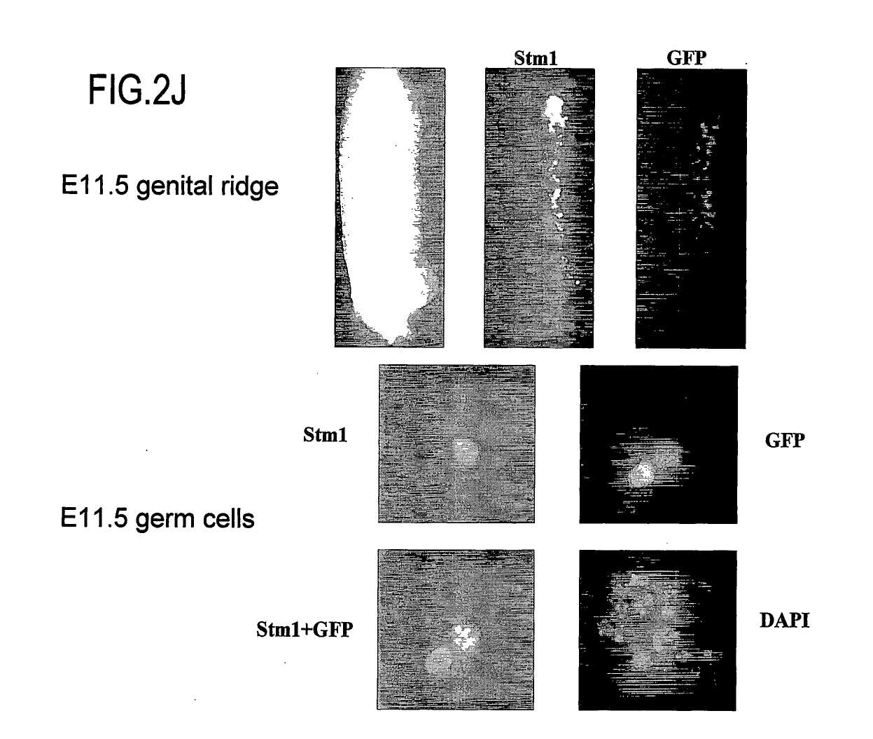

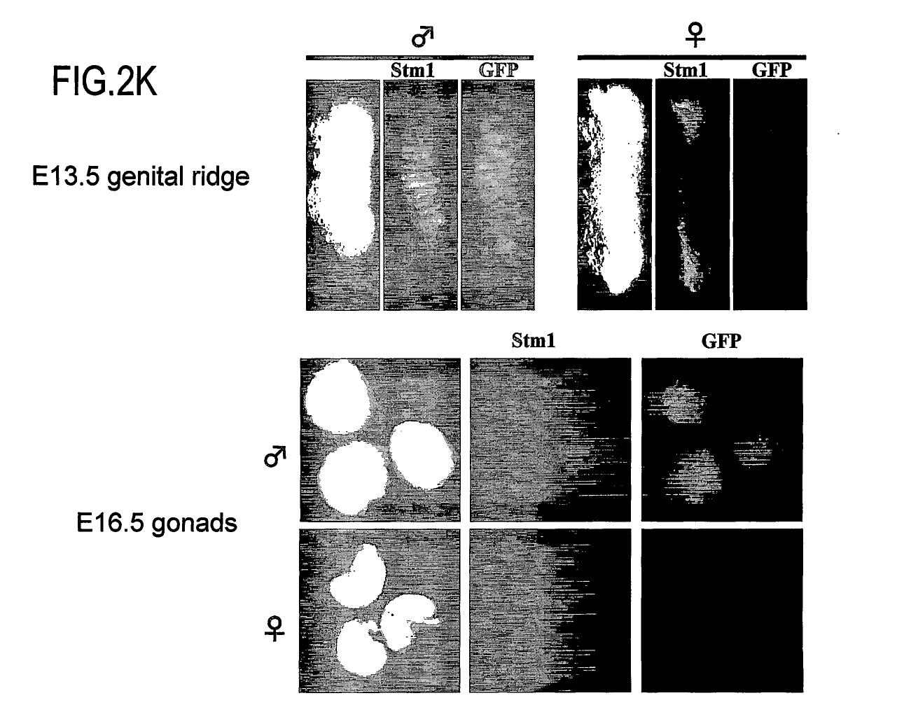

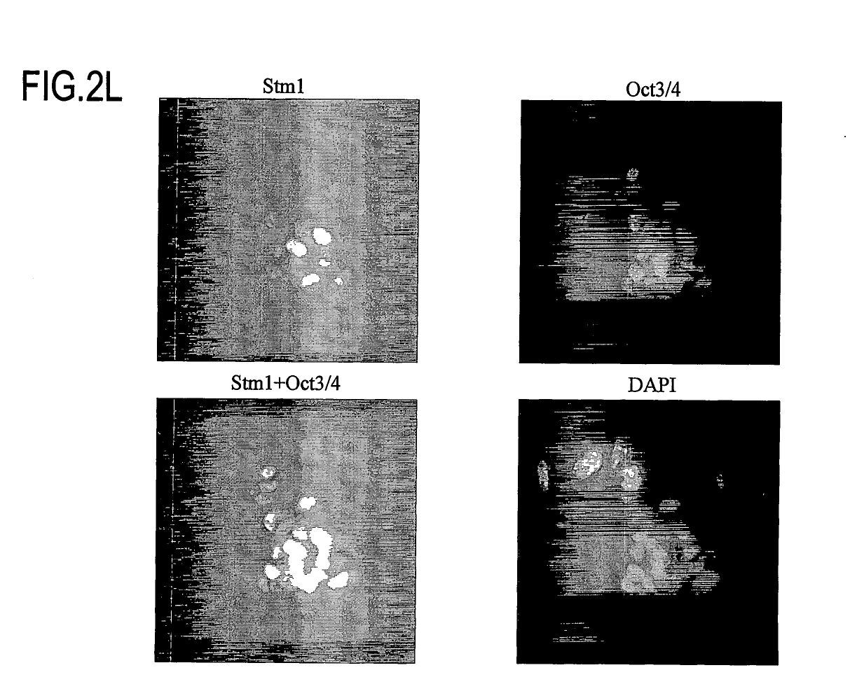

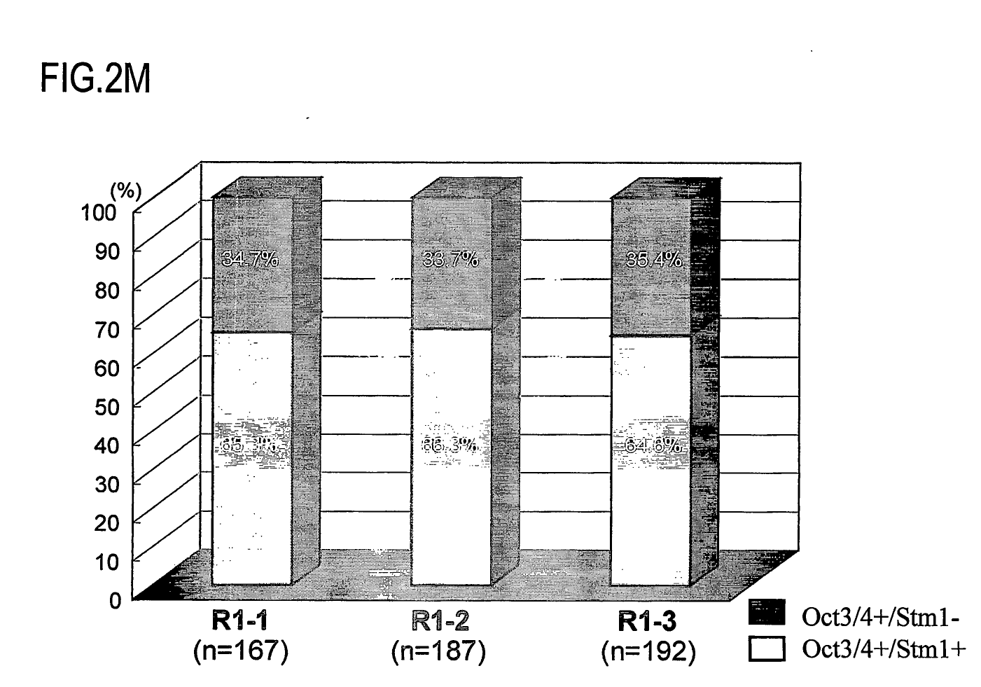

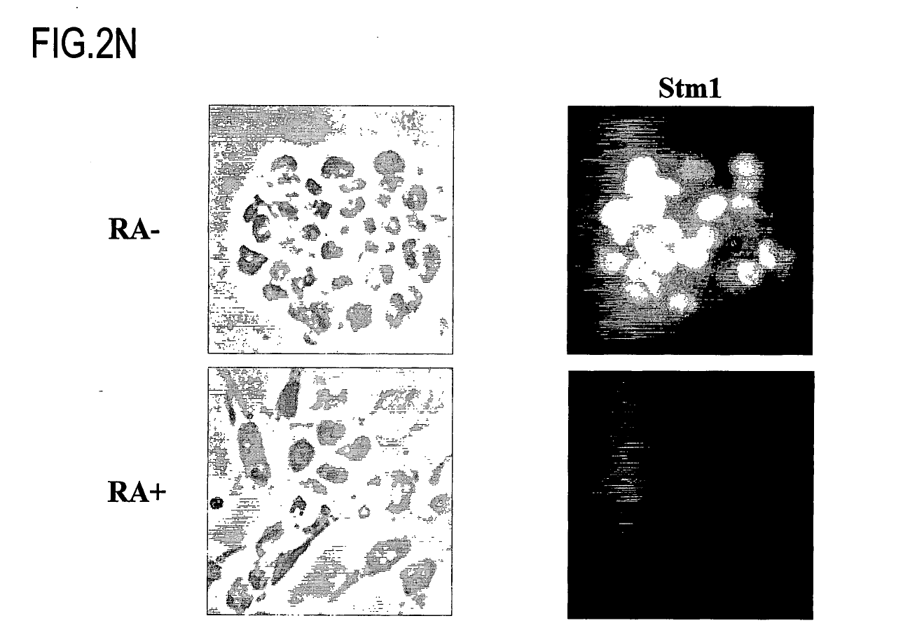

(Expression Pattern of Stm Gene) (FIGS. 1 and 2)

The present inventors identified the Stm gene as a gene which is expressed in ES (Embryonic Stem) cells and EG (Embryonic Germ) cells but which is not expressed in 12.5-day-old embryos, by subtraction of mRNA of ES cells and EG cells (FIG. 1B). Stm is an about 2.1-kb gene characterized by a homeodomain, a B2 repeat sequence, and a W-rich region (FIG. 1A). As a result of RT-PCR analysis using total RNA recovered from tissues of adults, expression of Stm was not found in any tissue. That is, expression of Stm had a very high characteristic to undifferentiated cells (ES cells and EG cells) (FIG. 1C). Transient expression of a myc-tagged Stm construct was performed in embryonic stem cells, and the location was detected using anti-myc antibodies. The localization of Stm was revealed to be in nuclei (FIG. 1D). This fact suggests a possibility that Stm functions as a transcription factor having a homeobox. Specific expression patterns were analyzed in early embryos and embryonic gonads by RT-PCR (FIG. 2). Expression was detected by using primers F2-R2 sandwiching a homeodomain (FIG. 2A). E6.5-E18.5 (i.e., embryos immediately after implantation to immediately before birth) were analyzed. As a result, expression was observed until E7.5-day-old embryos containing undifferentiated cells, and thereafter, expression rapidly disappeared (FIG. 2A). This seems to be associated with rapid disappearance of totipotency after E7.5. Expression was slightly observed after E8.5-day-old embryos which were highly developed. It was found that the expression pattern of Stm1 seemed to be different from that of Oct3/4. Particularly, Stm1 was not expressed in unfertilized eggs. Therefore, it was demonstrated that expression of a Stm1 gene of the present invention is closely correlated with pluripotency and totipotency. Although both Stm1 and Oct3/4 were expressed in morulae and blastocysts for embryos before implantation, expression was detected in unfertilized eggs only for Oct3/4 and not for Stm1 (FIG. 2B). This indicates that expression of Stm1 is attributed to zygotic expression due to activation of the nucleus after fertilization. Next, to observe expression in germ cells, female and male gonads of E12.5-day-old embryos were analyzed. Both Stm1 and Oct3/4 were expressed similarly. In order to demonstrate that such expression was caused by germ cells, primordial germ cells were purified from gonads. The degree of purification of germ cells was measured using an antibody SSEA-1 against a surface antigen specific to germ cells. 300 or more cells were analyzed. As a result, 95% or more of the cells were found to be SSEA-1 positive (FIG. 2C; developed red). Expression of Stm1 was positive in these primordial germ cells, it was as with Oct3/4 (FIG. 2C, right; color development with DAPI).

It was investigated whether or not similar properties were possessed by Stm2, using a specific restriction enzyme. The result is shown in FIG. 2D. As can be seen from FIG. 2D, expression of Stm2 was not clear at any of the stages investigated (ES cell, E7.5, E12.5, and blastocyst), even though expression of Stm1 was significant.

Therefore, it was demonstrated that Stm1 exhibit expression patterns specific to undifferentiated cells, which are similar to that of the Oct3/4 gene, however, these patterns are not the same and are different in function.

(Preparation of Antibodies)

Next, the full length amino acid sequence of Stm1 was used to produce rabbit polyclonal anti-Stm1 gene product antibodies. STM1 protein in cells corresponding to the amount of RNA could be detected based on the amount of these antibodies (FIG. 2E).

Next, these antibodies were used to investigate localization of STM1 protein in undifferentiated cells. As a result, it was revealed that STM1 protein was localized in the nuclei of undifferentiated cells (FIG. 2F).

Mouse Stm antibodies created stained images similar to that of Oct3/4, while co-cultured feeder cells were not stained. Similarly to the mouse, it was demonstrated that STM1 was expressed in ES cells of human, monkey, and rat (FIG. 2G, upper column).

Next, samples containing both mouse ES cells and lymphocytes were subjected to staining with STM1 antibodies. As a result, only mouse ES cells were stained (FIG. 2G, middle column).

Next, it was shown that STM1 protein was localized in the nuclei of undifferentiated cells (FIG. 2G, lower column).

Next, STM1 antibody was used to analyze localization of STM1 protein in mouse early embryos in detail. As a result, expression was not observed until the morula stage (unfertilized eggs, the 8-cell stage, and the 16-cell stage; FIG. 2H). In addition, expression was observed only in extraembryonic germ layer (epiblast) on E6.5 and E7.5. On E8.5, expression considerably disappeared. On E9.5, expression was considerably weaker (FIG. 2I). On E6.5, portions in the vicinity of borders with extraembryonic tissues were strongly stained (FIG. 2I). On E7.5, portions in the vicinity of the tail primitive streak were strongly stained (FIG. 2I). As shown in FIGS. 2J and 2K, it was observed that expression was enhanced on E11.5 to E13.5. As shown in FIG. 2K, expression began decreasing again on E16.5. In addition, the state of expression of mouse ES cells is shown in FIG. 2L. The expression of Stm1 was also shown in ES cells. FIG. 2H shows distribution of Oct3/4+/Stm1+ and Oct3/4+/Stm1− cells in ES cells. As can be seen, the Oct3/4+/Stm1+ cells constituted about ⅔ of the total of cells, while the Oct3/4+/Stm1− constituted about ⅓ of the total of cells. It was demonstrated that ES cells included more undifferentiated cells and differentiated cells. As shown in FIG. 2N, the expression of Stm1 disappeared due to induction of differentiation by retinoic acid stimulus (concentration).

According to the above-described results, it was demonstrated that mRNA of Stm1 was expressed only in early embryos. Specifically, Stm1 was expressed in morulae and blastocysts. The expression was reduced on E8.5. The expression was observed in the genital ridge (E12.5), ES cells, EG cells, and EC cells. These results are summarized in FIG. 2O. The expression was not observed in unfertilized eggs and adult tissues. Therefore, it is suggested that Stm1 of the present invention has a function of maintaining an undifferentiated state and inhibition of differentiation to endoderm.

Taken together, these results show that the gene of the present invention is more region-specific to regions, which are undifferentiated and has pluripotency, than Oct3/4 which is known to be expressed in undifferentiated embryonic stem cells. Therefore, the gene of the present invention provides an effect of specifying an undifferentiated state or pluripotency (preferably, totipotency) with such a level of efficiency and precision that cannot be conventionally achieved.

### Example 6

### Recovery of Genomic DNA

Genomic DNA was extracted in accordance with a general protocol (Sambrook and Russell, 1989, Molecular cloning: A Laboratory manual, Cold Spring Harbor Laboratory Press, New York, USA). Specifically, cells were suspended in extraction buffer, followed by treatment with RNaseA at 37° C. for 1 hour and then with ProteinaseK at 37° C. overnight. Thereafter, phenol extraction was performed twice, followed by ethanol precipitation to recover DNA.

### Example 7

### Southern Blot Hybridization Analysis

Southern blot hybridization was performed in accordance with a general protocol (Southern et al., J. Mol. Biol., 98: 503-517). Specifically, 20 μg of genomic DNA extracted from embryonic stem cells was dissolved in water, followed by electrophoresis with 1% agarose gel. Thereafter, the genomic DNA was blotted from the gel to a Hybond-N+ membrane overnight. The blotted membrane was subjected to prehybridization at 42° C. for 2 hours and then hybridization overnight. The membrane was washed twice in 2×SSC/0.1% SDS at 65° C. and once in 0.1×SSC/0.1% SDS. Probes used were:

exon2F:

(SEQ ID NO. 21)

5′-CCTCTCCTCGCCCTTCCT-3′
and
exon2R:

(SEQ ID NO. 22)

5′-CTGCTTATAGCTCAGGTTCAG-3′.

Fragments obtained by PCR amplification of genomic DNA using a primer set were used. DNA, which was labeled with [α-32P] dCTP (Amersham Biosciences) RI label using Megaprimer DNA labeling system (Amersham Biosciences), was used as a probe.

(Identification of Stm Genes)

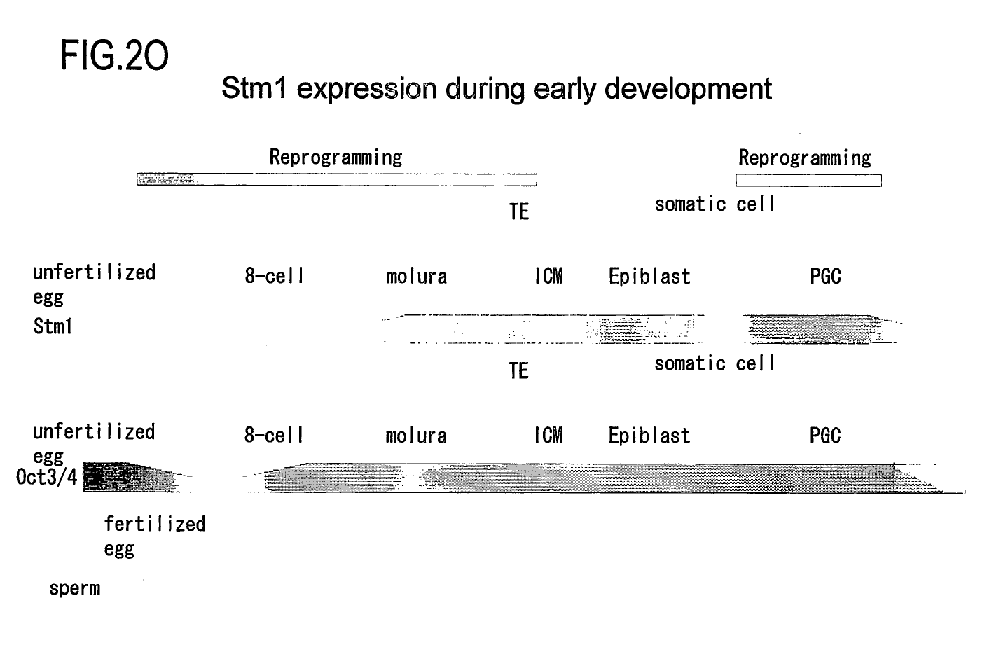

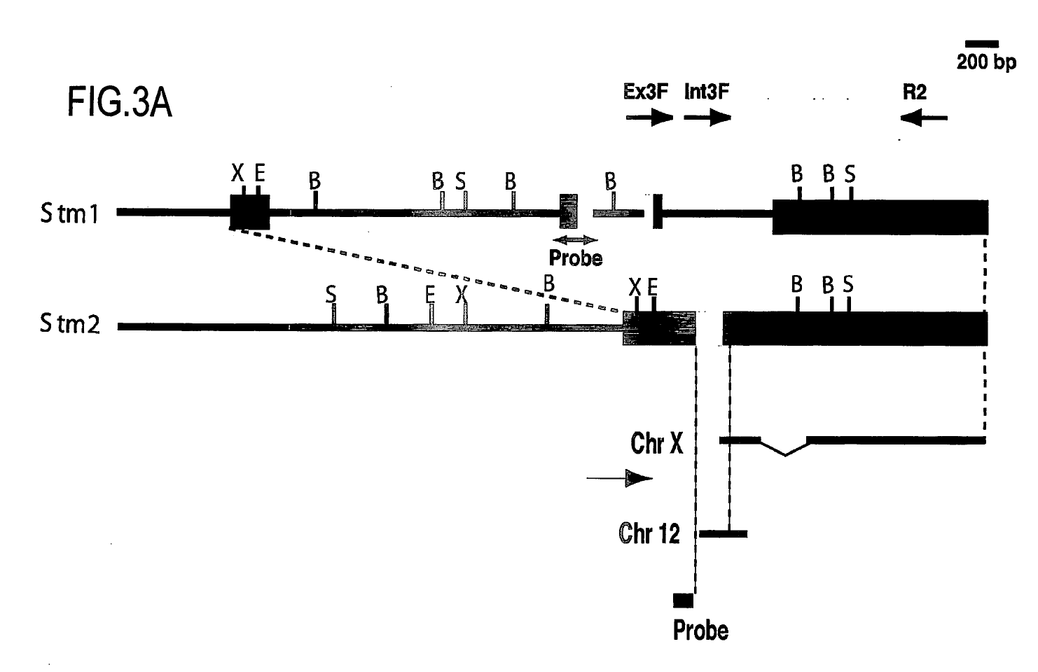

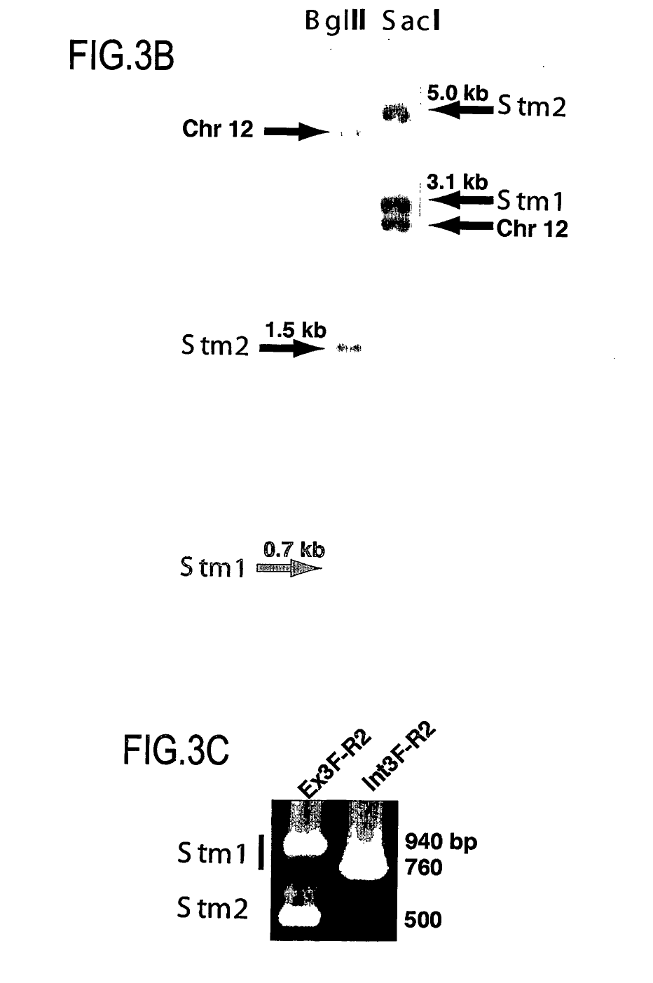

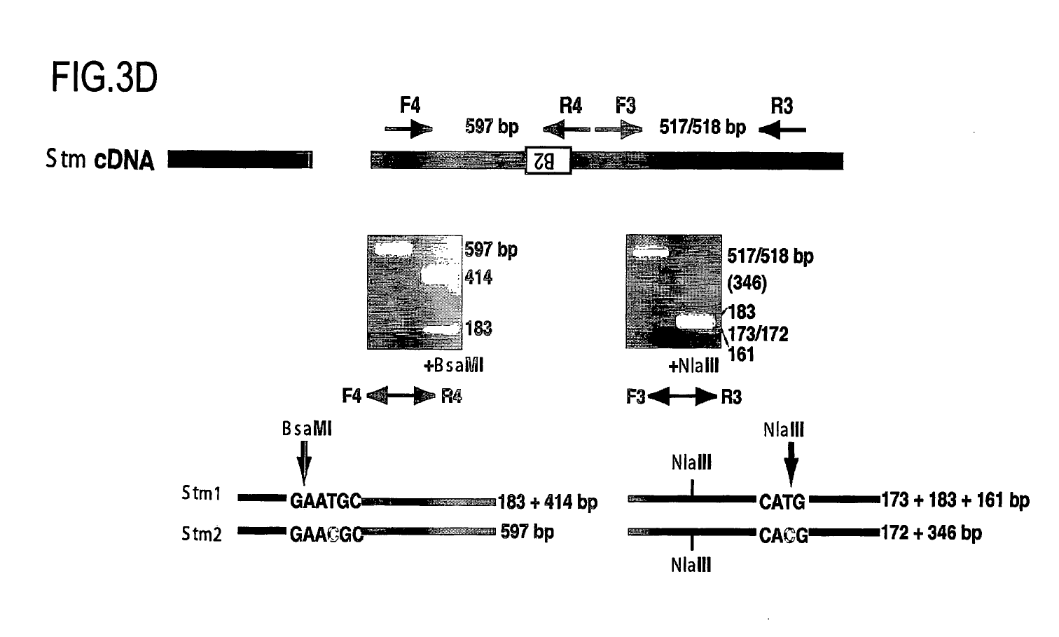

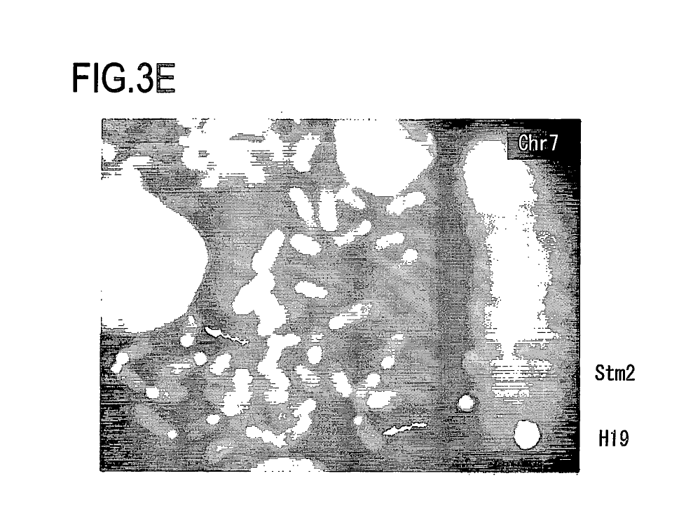

A homeobox region of Stm was used as a probe to perform Southern blot hybridization analysis on a mouse genome. As a result, a Stm1 gene consisting of 4 exons and an intronless Stm2 gene were identified (FIGS. 3A and 3B). The Stm1 gene was positioned on mouse Chromosome 6, while the Stm2 gene was positioned on Chromosome 7. Thus, these genes were mapped onto different loci. Precisely, the Stm2 gene was mapped onto 7E3 (FIG. 3E). The presence of Stm1 and Stm2 was also reconfirmed by genomic PCR analysis using Ex3F-R2 and Lnt3F-R2 primers (FIG. 3C). Computer database analysis found a partial homologous region to the homeodomain on Chromosome 12 and the X chromosome. To distinguish Stm1 from Stm2 in terms of gene expression, the sequences of cDNA regions on the genomes of Stm1 and Stm2 were compared. As a result, Stm1 matched Stm2 in 95% or more of the base sequence. The origin of Stm was determined by digesting RT-PCR products of Stm with restriction enzymes recognizing different base sequences. A F4-R4 primer set was used to amplify a 5′ side of a transcription product. Products of Stm1 are divided into 183-bp and 414-bp fragments by digesting with BsaMI enzyme, while a product of Stm2 is not digested. Since it was demonstrated that all RT-PCR products were digested, only the product of Stm1 was shown to be actually expressed (FIG. 3D). Similarly, when a 3′ side of a transcription product amplified with a F3-R3 primer set was digested with NlaIII enzyme, only DNA fragments derived from Stm1 were detected (FIG. 3D). Products of Chromosome 12 were similarly analyzed. No expression was observed. According to these results, Stm1 and Stm2 encode RNA having a very similar sequence, however, only Stm1 was actually transcribed into RNA. It was concluded that Stm2 is a pseudogene of Stm1. Transcription products detected in FIGS. 1 and 2 were analyzed with similar restriction enzymes. As a result, it was confirmed that all transcripts were derived from Stm1.

### Example 8

### Genomic Polymorphism Analysis

Genomic DNA was extracted from embryonic stem cells derived from Mus musuculus domesticus (general experimental mouse) and M.m.molossinus (as an experimental wild-type mouse) which are subspecies, followed by PCR amplification with the aforementioned F1 and R1 primers. The product was subjected to TA cloning with pGEM-T Easy vector system, and sequenced in opposite directions by a sequencing reaction using M13 forward and M13 reverse primers. Sequencing was performed using a capillary sequencer CEQ 2000XL DNA Analysis System (BECKMAN COULTER). These subspecies were compared in their base sequence data to determine the origin of one sequence distinguished from the other sequence. In addition, RT-PCR products obtained using F1 and R1 primers were cut with a restriction enzyme SnaBI, and the origin of the product was determined based on a difference in sensitivity due to a difference in base sequence at the SnaBI recognition site.

### Example 9

### Analysis on Expression of Stm1 and Stm2

The presence of Stm2, which is a pseudogene of Stm1, was confirmed by PCR where genomic DNA was used as a template and the following three primers were combined:

Ex3F:

(SEQ ID NO. 23)

5′-GTGGTTGAAGACTAGCAATGG-3′,
Int3F:

(SEQ ID NO. 24)

5′-CTATGGCTGTTGGGTATGGA-3′,
and R2.

PCR was performed under the following conditions: Incubation at 94° C. for 5 minutes; 30 cycles of 94° C. for 30 seconds, 60° C. for 30 seconds, and 72° C. for 1 minute; and finally incubation at 72° C. for 5 minutes. In the case of the combination of Ex3F-R2 primers, Stm1 and Stm2 had products of different sizes. In the case of the combination of Lnt3F-R2 primers, only Stm1 was detected. It was shown that both genes were present in the genome.

In order to distinguish expression of Stm1 from expression of Stm2, PCR amplification was performed where genomic DNA extracted, from embryonic stem cells of M.m.domesticus was used as a template, and the following primer set was used:

F3:

(SEQ ID NO. 25)

5′-CTTTGAACTAGCTCTGCAGA-3′
and
R3:

(SEQ ID NO. 26)

5′-TGAACTTATTGCATATCTGAG-3′;
F4:

(SEQ ID NO. 27)

5′-CAGGGCTATCTGGTGAACG-3′
and
R4:

(SEQ ID NO. 28)

5′-GAGCACCCGACTGCTCTTC-3′.

The F3-R3 and F4-R4 products were cloned and sequenced to determine their base sequences which were in turn compared. The origin of the product could be determined based on a difference in base sequence between the Stm1 and Stm2 products. Next, total RNA of embryonic stem cells was used as a template to clone and sequence RT-PCR products of F3-R3 and F4-R4. As a result, it was revealed that the resultant transcription product was expressed by the Stm1 gene. By cutting RT-PCR products of F3-R3 and F4-R4 with restriction enzymes NlaIII and BsaMI, respectively, the products were confirmed to be transcription products of Stm1.

### Example 10

### Stm1 as Marker Gene for Reprogramming

Experiments, such as cell fusion of a somatic cell and an embryonic stem cell and nuclear transplantation of the nucleus of a somatic cell into an enucleated unfertilized egg, have revealed that the nuclei of somatic cells are reprogrammed so that they can behave as undifferentiated cells do. It was empirically demonstrated that the former technique was effective for production of cloned cells, while the latter technique was effective for production of cloned individuals. However, the mechanism for reprogramming has not been elucidated. The Stm1 gene specific to an undifferentiated cell may be applied to at least two applications as follows: 1) a marker for reprogramming of a somatic cell nucleus into an undifferentiated cell nucleus; and 2) elucidation of the reprogramming mechanism by comparing with the Oct3/4 gene. For application 1), cell fusion and nuclear transplantation experiments were performed.

(Cell Fusion and Nuclear Transplantation)

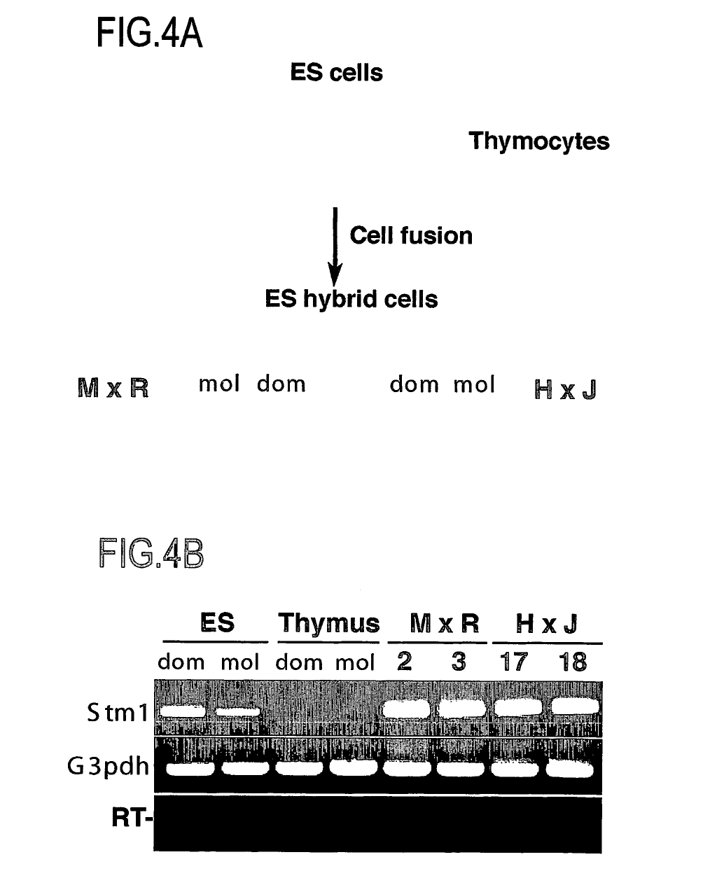

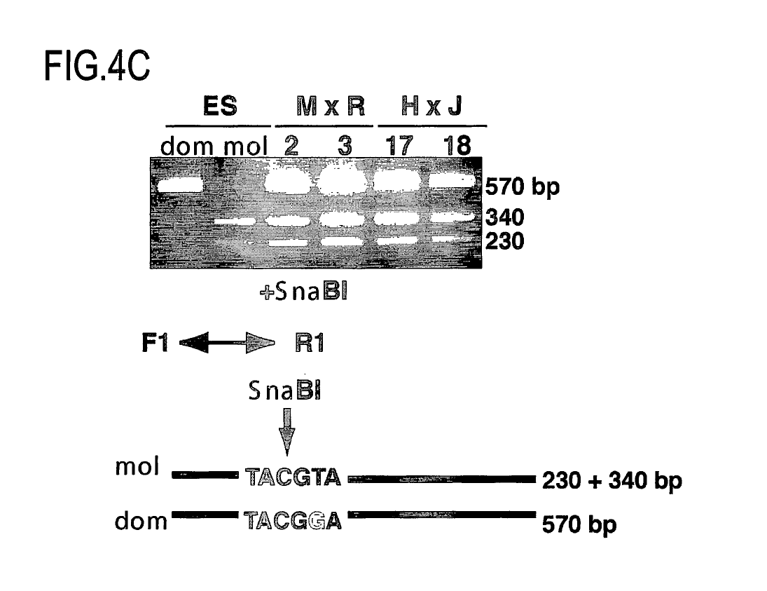

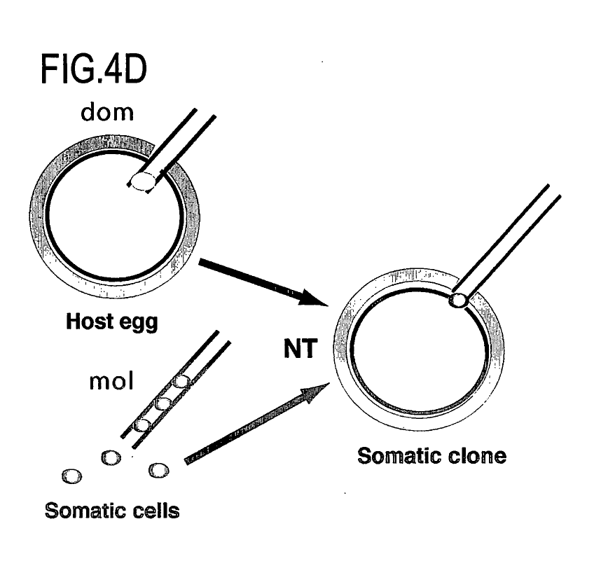

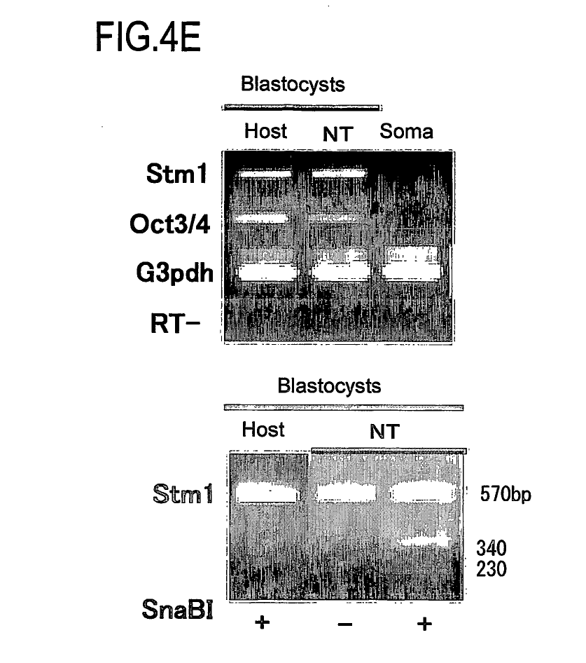

In cell fusion experiments, in order to distinguish the Stm1 transcription products in a somatic cell nucleus from the Stm1 transcription products in an embryonic stem cell nucleus, embryonic stem cells and somatic cells derived from Mus musculus molossinus were used. The Molossinus-derived embryonic stem cell was newly established in our laboratory. The Molossinus genome has a number of base sequence polymorphisms as compared with mouse M.m.domesticus. Therefore, by using a fusion cell of molossinus and domesticus, the origin of transcription products can be determined. A method for producing a fusion cell is shown in FIG. 4A. A fusion cell using a Molossinus-derived embryonic stem cell is represented by M×R, while a fusion cell using a Molossinus-derived somatic cell is represented by H×J. Stm1 was not expressed in the somatic cells (thymus cells). On the other hand, Stm1 was expressed in the embryonic stem cells. It was found that Stm1 was expressed in all M×R and H×J fusion cell clones (FIG. 4B). In order to confirm that the Stm1 gene is expressed both in an embryonic stem cell nucleus and in a reprogrammed somatic cell nucleus, RT-PCR products using the F1-R1 primer set were digested with the SnaBI restriction enzyme. Molossinus-derived transcription products were sensitive to SnaBI digestion, so that a 570-bp band was divided into 230-bp and 340-bp bands. In contrast, domesticus transcription products were not digested with SnaBI, so that a 570 bp band remained. In the M×R and H×J fusion cells, both a band which was digested with SnaBI and a band which was not digested with SnaBI were detected. Therefore, it was demonstrated that Stm1 was transcribed both in the embryonic stem cell nucleus and in the reprogrammed somatic cell nucleus (FIG. 4C). The Stm1 gene can be used as a marker for a reprogrammed somatic cell nucleus.

(Marker for Undifferentiated State)

In order to determine whether Stm1 can be used as a marker gene for a reprogrammed somatic cell in nuclear transplantation, the following experiment was performed. The nucleus was removed from an unfertilized egg of (B6×CBA) F1 mouse (domesticus), into which the nucleus of fibroblasts from (B6×JF1 (molossinus)) F1 fetuses was in turn transplanted (FIG. 4D). In the resultant cloned blastocyst, expression of Stm1 was examined. Stm1, which had not been expressed in the embryonic fibroblasts, was re-expressed in the cloned blastocyst (FIG. 4E). In addition, it was shown that somatic cell-derived Stm1 was expressed in the cloned blastocysts. The origin of transcription products of Stm1 was confirmed based on a difference in sensitivity to SnaBI digestion. In cloned blastocysts, molossinus-derived Stm1 was expressed. The experiments on cell fusion of a somatic cell and an embryonic stem cell and nuclear transplantation of a somatic cell nucleus demonstrated that Stm1 is useful as a marker for nuclear reprogramming.

Next, expression of the Stm1 gene in cloned blastocysts into which the nucleus of a somatic cell was transplanted was examined. cDNA synthesized from mRNA derived from a (mol×dom) F1 cloned blastocyst was amplified by PCR using F1-R1 primers (FIG. 1A). As a positive control, the Oct3/4 gene was used. The resultant Stm1 products were digested with restriction enzyme SnaBI. A mol-derived product is sensitive to SnaBI digestion, while a dom-derived product is resistant to SnaBI digestion. For F1 cloned blastocysts, not only dom-derived products but also mol-derived products were detected. It was demonstrated that somatic cell nucleus-derived Stm1 was reactivated by nuclear transplantation.

### Example 11

### Stm1 as Marker Gene for Tissue Stem Cell

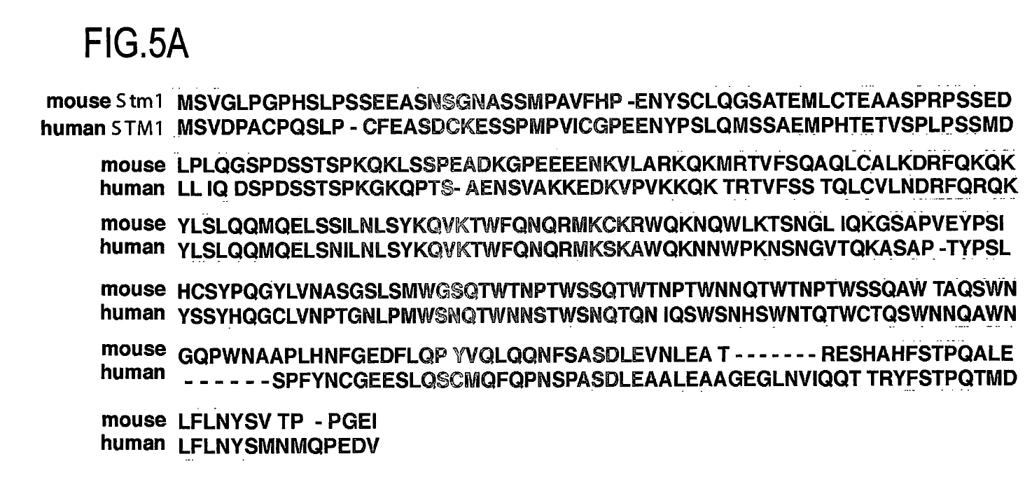

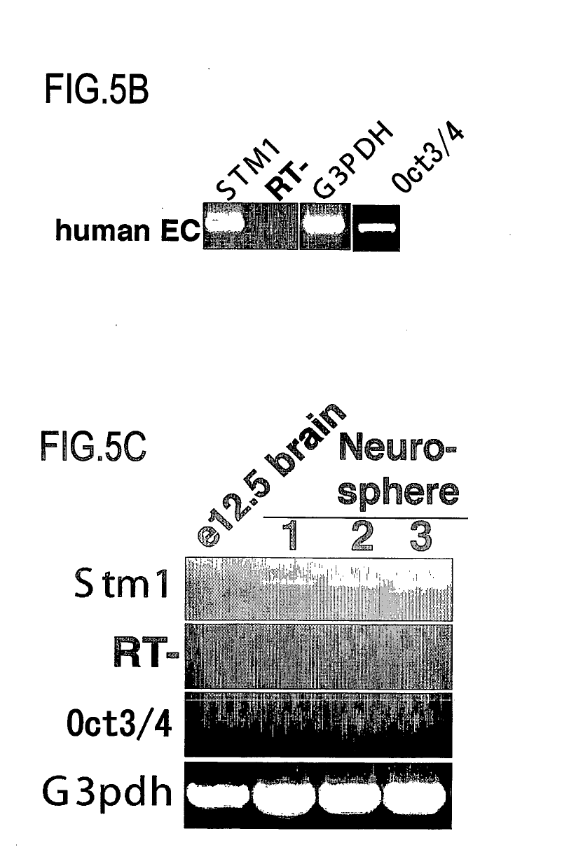

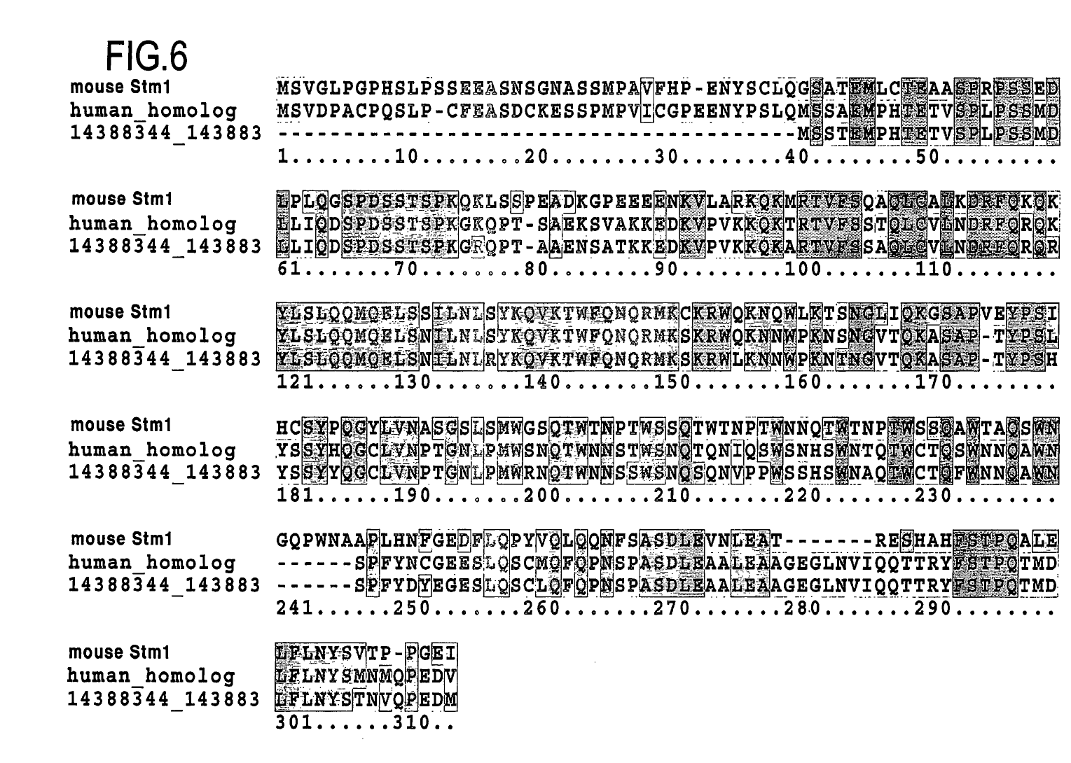

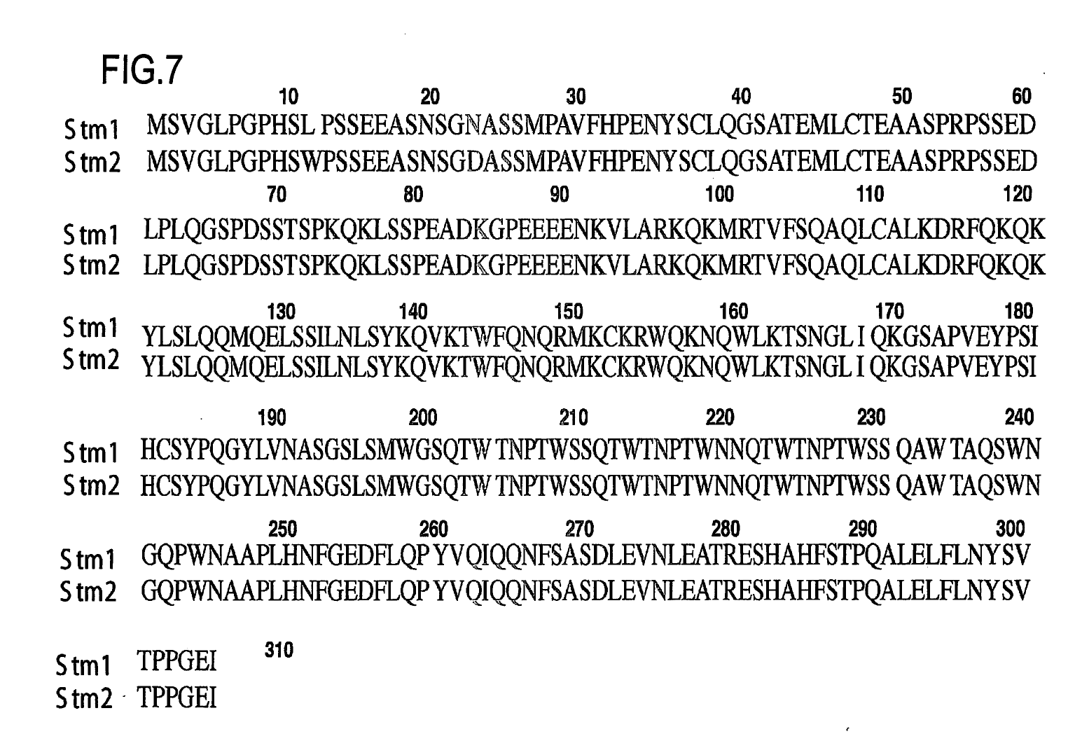

Tissue stem cells as well as embryonic stem cells have attracted attention for application to regenerative medicine. A tissue stem cell is a cell which is a source for supplying new cells associated with the metabolism of tissues. Tissue stem cells are considered to be present in each tissue. However, no method for establishing and purifying such cells has been achieved. A specific marker for purifing tissue stem cells was a hurdle. Only bone marrow interstitial tissue stem cells and MAPC have been reported as tissue stem cells in which Oct3/4 is expressed. As pluripotent stem cells, there are cerebral stem cells (NS; Neurosphere) as well as MAPC. However, expression of Oct3/4 in NS has not yet been reported. Expression of Stm1 was examined in MAPC-like cells and NS, so that although Oct3/4 was not expressed, expression of Stm1 was observed (FIG. 5). The aforementioned primer set can be used to detect the genomic DNA and RNA of Stm1 based on the size of products. Therefore, it is clear that RNA was detected. This suggests a possibility that the control of expression of Stm1 is independent of Oct3/4, and a possibility that Stm1 is located upstream of Oct3/4 and Stm1 serves as a marker gene for identifying an initial undifferentiated cell before expression of Oct3/4. In addition, homologs of Stm1 are also present in primates, such as cynomolgus monkey and human. Expression of Stm1 was also confirmed in cynomolgus monkey embryonic stem cells and human EC cells. These facts suggest a possibility of application of Stm1 to regenerative medicine.

### Example 12

### Isolation of Undifferentiated Cell

In Example 12, Stm1 was used to isolate stem cells in order to apply Stm1 to regenerative medicine.

Stm1 can be used as a marker for an undifferentiated state of all stem cells. Therefore, the fluorescent marker gene GFP was introduced under the control of the Stm1 promoter, and expression of Stm1 was monitored. Living tissue stem cells could be enriched from tissue cells or cultured cells thereof using expression of GFP as a marker. In addition, it is possible to select only highly pluripotent cells among other embryonic stem cells. Gene knockout experiments using homologous recombination have clarified that embryos lacking the gene function of Stm1 are mortal during early embryonic development. This suggests that Stm1 is essential for maintenance of an undifferentiated state.

Therefore, it was demonstrated that a promoter of the Stm1 gene can be used as a marker for an undifferentiated cells.

### Example 13

### Function of Stm Gene Variant

A Stm1 gene is characterized by a homeodomain, a B2 repeat sequence and a W-rich region. Point mutations (e.g., substitution of A for T at position 500 in SEQ ID NO. 3 (mouse Stm1 gene), and substitution of T for A at position 800, substitution of A for T at position 1200) were introduced into these base sequences so that their encoded amino acid sequences were changed. Based on these mutations, the functions of the regions were examined. The regions were partially knocked out (e.g., positions 500 to 550, positions 800 to 850, and positions 1200 to 1250) to examine their functions.

For example, Stm1 is a protein which is localized in nuclei and has a homeodomain, and therefore, it is inferred that Stm1 has a function of suppressing expression of a protein inducing differentiation. For the above-described mutants, if the deletion of a homeodomain destroys the mechanism for maintaining an undifferentiated state of a cell, it will be demonstrated that the homeodomain plays an important role in control of the mechanism.

### Example 14

### Function of Stm Gene

Conditional knockout experiments are conducted to analyze functions of specific undifferentiated tissue cells, such as early embryos and germ cells. Most functions of the STM1 protein are unknown. However, the STM1 protein is demonstrated as having a function of regulating expression of a downstream gene under the control of STM1 or Stm1 to alter a cell into an undifferentiated state (i.e., rejuvenation).

### Example 15

### Identification of Promoter Sequence

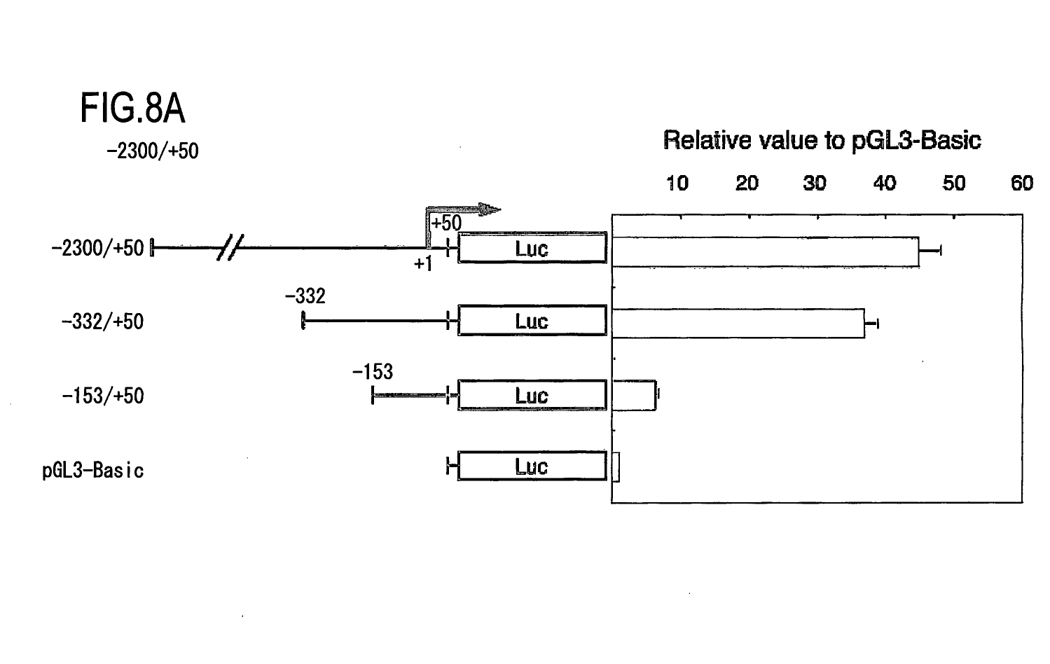

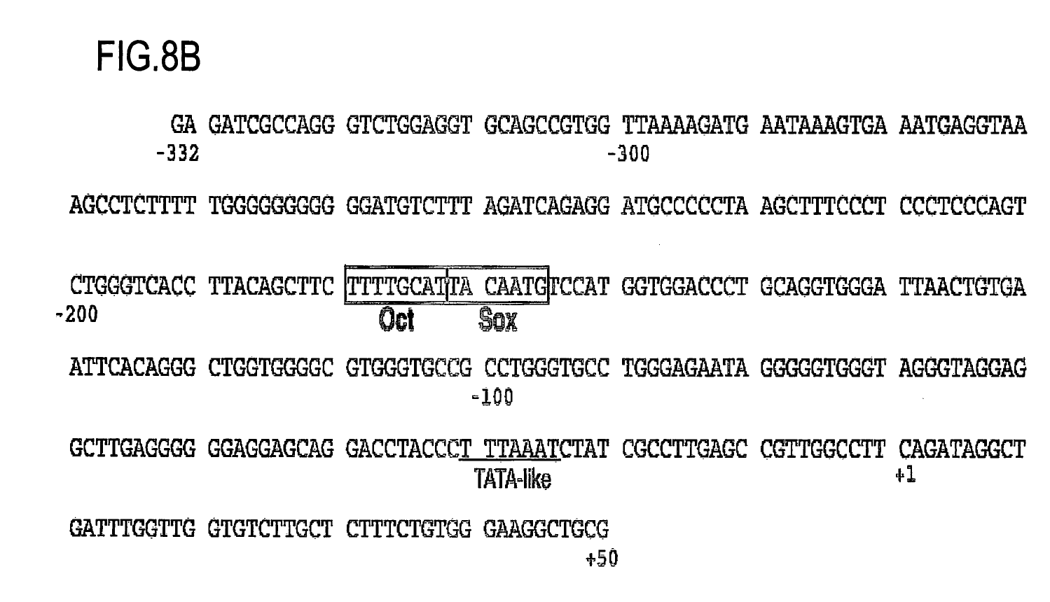

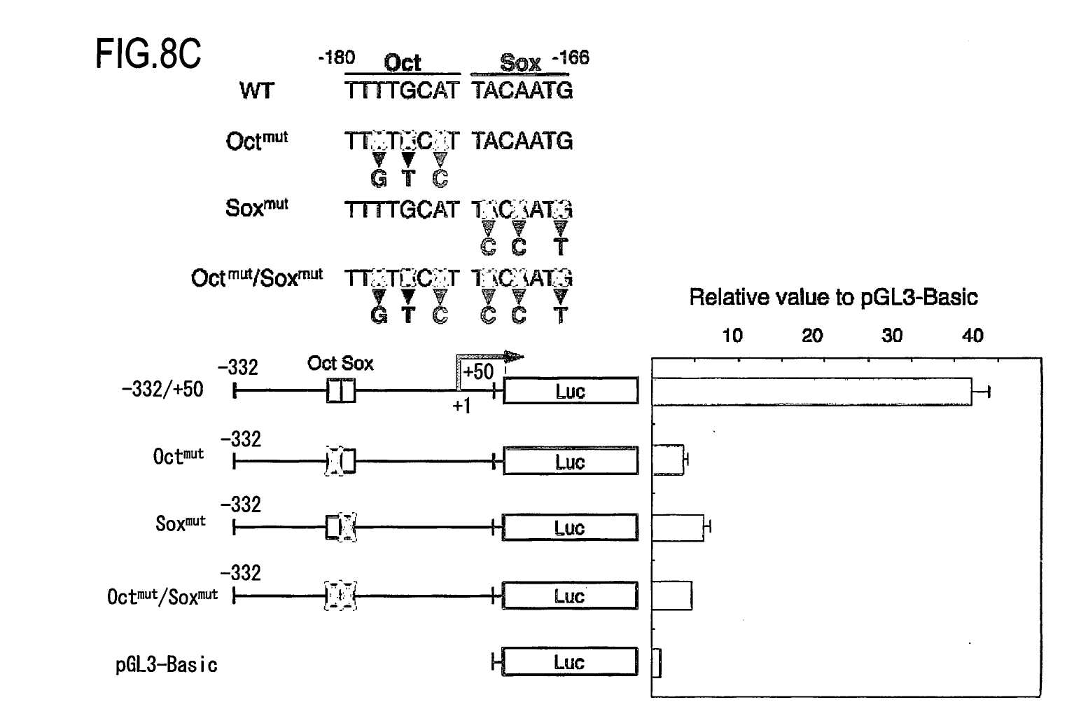

Next, a promoter sequence of Stm1 was identified. Short portions were removed from 2300 bp upstream of the transcription start site (−2300 bp) toward the 5′ end. A luciferase gene was linked to 5′ upstream regions having different sizes to produce several constructs (FIG. 8A). A luciferase assay was performed. In the luciferase assay, the intensity of light emitted by luciferase was measured and evaluated. As a result, it was demonstrated a region of −332 bp to −153 bp from the transcription start site contains an element which controls transcription in a positive manner.

Next, in order to identify such an element, we focused on a site, in which an Octamer binding domain (Oct motif) and a Sox binding domain (Sox motif) are contiguously present, among transcription agent-binding sequences present in the region of −332 bp to −153 bp from the transcription start site (FIG. 8B).

A sequence of three bases was introduced into each domain or both. Thereafter, luciferase activity was compared. The result is shown in FIG. 8C. According to the result, it was demonstrated that the above-described site was important for controlling transcription activity and both of the above-described domains were required.

In the site, a minimum essential portion for a promoter was revealed to be positions −180 to −166 (TTTTGCATTACAATG) in SEQ ID NO. 32 which sets forth positions −332 to +50 (FIG. 8B).

Although certain preferred embodiments have been described herein, it is not intended that such embodiments be construed as limitations on the scope of the invention except as set forth in the appended claims. All patents, published patent applications and publications cited herein are incorporated by reference as if set forth fully herein.

### INDUSTRIAL APPLICABILITY

Accurate determination of stem cells was achieved, which had not been realized with conventional agents. Therefore, the present invention can be used for various applications, such as accurate determination and purification of stem cells, such as ES cells and the like, and is highly useful.

1. A nucleic acid molecule, comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity.

2. A nucleic acid molecule according to claim 1, wherein the nucleic acid molecule is at least 10 contiguous nucleotides in length.

3. A nucleic acid molecule according to claim 1, wherein the nucleic acid molecule has a sequence different from a sequence set forth in SEQ ID NO. 7 or 9 or a corresponding sequence in a corresponding nucleic acid sequence of Stm2 in at least one position in SEQ ID NO. 1, 3, 5 or 29.

4. A nucleic acid molecule according to claim 3, wherein a portion having the different sequence may be digested with a restriction enzyme.

5. A nucleic acid molecule according to claim 1, comprising a sequence set forth in SEQ ID NO. 1, 3, 5 or 29.

6. A nucleic acid molecule, comprising:
(a) a polynucleotide having a base sequence of positions 1037 to 1607 or 244 to 1126 set forth in SEQ ID NO. 3 or a base sequence in corresponding positions, or a fragment thereof;
(b) a polynucleotide hybridizable to the polynucleotide of (a) under stringent conditions, and encoding a polypeptide biological activity; or
(c) a polynucleotide consisting of a base sequence having at least 70% identity to any one of the polynucleotides of (a) to (b) or a complementary sequence thereof, and encoding a polypeptide having biological activity.

7. An agent, which is specific to a nucleic acid molecule according to claim 1.

8. An agent according to claim 7, wherein the agent does not react specifically with a nucleic acid molecule of a Stm2 gene having a sequence set forth in SEQ ID NO. 7 or 9, or a corresponding nucleic acid sequence thereof.

9. An agent according to claim 7, wherein the agent is selected from the group consisting of a nucleic acid molecule, a polypeptide, a lipid, a sugar chain, a low molecular weight organic molecule, and a composite molecule thereof.

10. An agent according to claim 7, wherein the agent is a nucleic acid molecule of at least 8 contiguous nucleotides in length.

11. An agent according to claim 7, wherein the agent is a nucleic acid molecule and is used as a primer.

12. An agent according to claim 7, wherein the agent is used as a probe.

13. An agent according to claim 7, wherein the agent is labeled or labelable.

14. An agent according to claim 13, wherein the label is used in a technique selected from the group consisting of fluorescence, phosphorescence, chemiluminescence, radiation, enzyme-substrate reaction, and antigen-antibody reaction.

15. A polypeptide, comprising:
(a) a polypeptide consisting of an amino acid sequence set forth in SEQ ID NO. 2, 4, 6 or 30, or a fragment thereof;
(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;
(c) a polypeptide encoded by a spliced mutant or alleic mutant of a base sequence set forth in SEQ ID NO. 1, 3, 5 or 29;
(d) a polypeptide being a species homolog of an amino acid sequence set forth in SEQ ID NO. 2, 4, 6 or 30; or
(e) a polypeptide having at least 70% identity to anyone of the polypeptides of (a) to (d) and having biological activity.

16. A polypeptide according to claim 15, wherein the polypeptide has an amino acid sequence having at least 3 contiguous amino acids.

17. A polypeptide according to claim 15, wherein the polypeptide has a sequence different from a sequence set forth in SEQ ID NO. 8 or 10 or a corresponding sequence in a corresponding amino acid sequence of Stm2 in at least one position in SEQ ID NO. 2, 4, 6 or 30.

18. A polypeptide according to claim 17, wherein a portion having the different sequence may be digested with a restriction enzyme.

19. A polypeptide, comprising:
(a) a polypeptide consisting of an amino acid sequence of positions 157 to 218 (homeodomain), positions 261 to 301 (W-rich region), or positions 399 to 455 (B2 repeat sequence region) set forth in SEQ ID NO. 4 or an amino acid sequence in corresponding positions, or a fragment thereof;
(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;
(c) a polypeptide having at least 70% identity to anyone of the polypeptides of (a) to (b) and having biological activity.

20. An agent, which is specific to a nucleic acid molecule according to claim 15.

21. An agent according to claim 20, wherein the agent is selected from the group consisting of a nucleic acid molecule, a polypeptide, a lipid, a sugar chain, a low molecular weight organic molecule, and a composite molecule thereof.

22. An agent according to claim 20, wherein the agent is an antibody or a derivative thereof.

23. An agent according to claim 20, wherein the agent is used as a probe.

24. An agent according to claim 20, wherein the agent is labeled or labelable.

25. An agent according to claim 24, wherein the label is used in a technique selected from the group consisting of fluorescence, phosphorescence, chemiluminescence, radiation, enzyme-substrate reaction, and antigen-antibody reaction.

26. An expression cassette, comprising a nucleic acid molecule according to claim 1.

27. A vector, comprising a nucleic acid molecule according to claim 1.

28. A vector according to claim 27, further comprising a control sequence operably linked to the nucleic acid molecule.

29. A vector according to claim 28, wherein the control sequence induces expression of the nucleic acid molecule.

30. A vector according to claim 28, further comprising a sequence encoding a selectable marker.

31. A cell, comprising a nucleic acid molecule according to claim 1.

32. A cell, comprising a nucleic acid molecule according to claim 1 in a manner which allows for expression of the nucleic acid molecule.

33. A cell, comprising a nucleic acid molecule according to claim 1 in a manner which allows for expression of the nucleic acid molecule and having a desired genomic sequence.

34. An animal tissue, comprising a nucleic acid molecule according to claim 1.

35. An animal, comprising a nucleic acid molecule according to claim 1.

36. A composition, comprising a concentrated cell comprising a nucleic acid molecule according to claim 1.

37. A nucleic acid molecule, comprising a sequence of a promoter portion of a Stm gene.

38. A vector, comprising a nucleic acid molecule according to claim 37.

39. A vector according to claim 18, further comprising a sequence encoding a selectable marker.

40. A cell, comprising a nucleic acid molecule according to claim 37.

41. An animal tissue, comprising a nucleic acid molecule according to claim 37.

42. An animal, comprising a nucleic acid molecule according to claim 37.

43. A composition, comprising a concentrated cell comprising a nucleic acid molecule according to claim 37.

44. A composition for determining an undifferentiated state of a cell, comprising an agent capable of reacting specifically with a Stm gene or a Stm gene product.

45. A composition according to claim 44, wherein the Stm gene or Stm gene product is:
(A) a nucleic acid molecule comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity, or
(B) a polypeptide comprising:
(a) a polypeptide consisting of an amino acid sequence set forth in SEQ ID NO. 2, 4, 6 or 30, or a fragment thereof;
(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;
(c) a polypeptide encoded by a spliced mutant or alleic mutant of a base sequence set forth in SEQ ID NO. 1, 3, 5 or 29;
(d) a polypeptide being a species homolog of an amino acid sequence set forth in SEQ ID NO. 2, 4, 6 or 30; or
(e) a polypeptide having at least 70% identity to anyone of the polypeptides of (a) to (d) and having biological activity.

46. A composition according to claim 44, wherein the cell is a stem cell.

47. A composition according to claim 44, wherein the cell includes an embryonic stem cell, a pluripotent stem cell, a unipotent stem cell, and a tissue stem cell.

48. A composition according to claim 44, wherein the cell includes a tissue stem cell selected from the group consisting of a neural stem cell, a gonadal stem cell, a hematopoietic stem cell, an epidermic stem cell, and mesenchymal tissue stem cell.

49. A composition according to claim 44, wherein the cell is genetically modified or is not genetically modified.

50. A method for determining an undifferentiated state of a cell, comprising the steps of:
(I) providing a cell to be determined;
(II) contacting an agent capable of reacting specifically with a Stm gene or a Stm gene product with the cell; and
(III) detecting a specific reaction between the agent and the Stm gene or the Stm gene product to determine whether or not the Stm gene is expressed in the cell,
wherein expression of the Stm gene in the cell indicates that the cell is in an undifferentiated state.

51. A method according to claim 50, wherein the undifferentiated state is totipotency.

52. A method according to claim 50, wherein the Stm gene or the Stm gene product comprises:
(A) a nucleic acid molecule comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity, or
(B) a polypeptide comprising:
(a) a polypeptide consisting of an amino acid sequence set forth in SEQ ID NO. 2, 4, 6 or 30, or a fragment thereof;
(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;
(c) a polypeptide encoded by a spliced mutant or alleic mutant of a base sequence set forth in SEQ ID NO. 1, 3, 5 or 29;
(d) a polypeptide being a species homolog of an amino acid sequence set forth in SEQ ID NO. 2, 4, 6 or 30; or
(e) a polypeptide having at least 70% identity to anyone of the polypeptides of (a) to (d) and having biological activity.

53. A method according to claim 50, further comprising determining whether or not another stem cell marker is expressed.

54. A method according to claim 53, wherein the other stem cell marker includes Oct3/4.

55. A method according to claim 50, wherein the Stm gene is a Stm1 gene.

56. A method according to claim 55, wherein the Stm1 gene comprises a sequence set forth in SEQ ID NO. 1, 3, 5 or 29.

57. A method for preparing a cell in an undifferentiated state, comprising the steps of:
(I) providing a sample known or suspected of containing the cell in an undifferentiated state;
(II) contacting an agent capable of reacting specifically with a Stm gene or a Stm gene product with the sample;
(III) determining whether or not the Stm gene is expressed in the cell in the sample; and
(IV) isolating or concentrating the cell in which the Stm gene is expressed.

58. A method according to claim 57, wherein the undifferentiated state is totipotency.

59. A method according to claim 57, wherein the Stm gene or Stm gene product comprises:
(A) a nucleic acid molecule comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity, or
(B) a polypeptide comprising:
(a) a polypeptide consisting of an amino acid sequence set forth in SEQ ID NO. 2, 4, 6 or 30, or a fragment thereof;
(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;
(c) a polypeptide encoded by a spliced mutant or alleic mutant of a base sequence set forth in SEQ ID NO. 1, 3, 5 or 29;
(d) a polypeptide being a species homolog of an amino acid sequence set forth in SEQ ID NO. 2, 4, 6 or 30; or
(e) a polypeptide having at least 70% identity to anyone of the polypeptides of (a) to (d) and having biological activity.

60. A method for preparing a cell in an undifferentiated state, comprising the steps of:
(I) providing the cell; and
(II) inducing expression of a Stm gene in the cell.

61. A method according to claim 60, wherein the undifferentiated state is totipotency.

62. A method according to claim 60, wherein the Stm gene comprises:
(A) a nucleic acid molecule comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity.

63. A method for isolating and/or growing and/or concentrating a cell in an undifferentiated state, comprising the steps of:
(I) providing a cell;
(II) introducing a Stm gene or a Stm gene promoter into the cell; and
(III) selecting the cell in which the Stm gene or the Stm gene promoter is expressed.

64. A method according to claim 63, wherein the undifferentiated state is totipotency.

65. A method according to claim 63, wherein the Stm gene or the Stm gene promoter comprises:
(A) a nucleic acid molecule comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity, or
(B) a sequence comprising a promoter portion of a Stm1 gene.

66. A kit for determining a differentiated state of a cell, comprising:
(a) an agent capable of reacting specifically with a Stm gene or a Stm gene product; and
(b) means for determining whether or not the Stm gene is expressed in the cell.

67. A kit according to claim 66, wherein the differentiated state is pluripotency.

68. A kit according to claim 66, wherein the differentiated state is totipotency.

69. A kit according to claim 66, wherein the Stm gene or Stm gene product comprises:
(A) a nucleic acid molecule comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity, or
(B) a polypeptide comprising:
(a) a polypeptide consisting of an amino acid sequence set forth in SEQ ID NO. 2, 4, 6 or 30, or a fragment thereof;
(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;
(c) a polypeptide encoded by a spliced mutant or alleic mutant of a base sequence set forth in SEQ ID NO. 1, 3, 5 or 29;
(d) a polypeptide being a species homolog of an amino acid sequence set forth in SEQ ID NO. 2, 4, 6 or 30; or
(e) a polypeptide having at least 70% identity to anyone of the polypeptides of (a) to (d) and having biological activity.

70. A kit according to claim 66, further comprising means for determining whether or not another stem cell marker is expressed.

71. A kit according to claim 70, wherein the other stem cell marker includes Oct3/4.

72. A kit according to claim 66, wherein the Stm gene is a Stm1 gene.

73. A kit for preparing a cell in an undifferentiated state, comprising:
(I) an agent capable of reacting specifically with a Stm gene or a Stm gene product; and
(II) means for determining whether or not the Stm gene is expressed in the cell.
(III) isolating or concentrating the cell in which the Stm gene is expressed.

74. A kit according to claim 73, wherein the undifferentiated state is totipotency.

75. A kit according to claim 73, wherein the Stm gene or Stm gene product comprises:
(A) a nucleic acid molecule comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity, or
(B) a polypeptide comprising:
(a) a polypeptide consisting of an amino acid sequence set forth in SEQ ID NO. 2, 4, 6 or 30, or a fragment thereof;
(b) a polypeptide having an amino acid sequence set forth in SEQ ID NO. 2, 4, 6 or 30, or a fragment thereof, wherein at least one amino acid in the sequence has a mutation selected from the group consisting of substitution, addition, and deletion, and wherein the variant polypeptide has biological activity;
(c) a polypeptide encoded by a spliced mutant or alleic mutant of a base sequence set forth in SEQ ID NO. 1, 3, 5 or 29;
(d) a polypeptide being a species homolog of an amino acid sequence set forth in SEQ ID NO. 2, 4, 6 or 30; or
(e) a polypeptide having at least 70% identity to any one of the polypeptides of (a) to (d) and having biological activity.

76. A kit for preparing a cell in an undifferentiated state, comprising:
(I) means for inducing expression of a Stm gene in the cell.

77. A kit according to claim 76, wherein the undifferentiated state is totipotency.

78. A kit according to claim 76, wherein the Stm gene comprises:
(A) a nucleic acid molecule comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity.

79. A kit for preparing a cell in an undifferentiated state, comprising:
(I) a vector containing a Stm gene operably linked to a control sequence.

80. A kit according to claim 79, wherein the undifferentiated state is totipotency.

81. A kit according to claim 79, wherein the Stm gene comprises:
(A) a nucleic acid molecule comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity.

82. A kit for isolating and/or growing and/or concentrating a cell in an undifferentiated state, comprising:
(I) a Stm gene or a Stm gene promoter;
(II) means for introducing the Stm gene or the Stm gene promoter into the cell; and
(III) means for selecting the cell in which the Stm gene or the Stm gene promoter is expressed.

83. A kit according to claim 82, wherein the undifferentiated state is totipotency.

84. A kit according to claim 82, wherein the Stm gene comprises:
(A) a nucleic acid molecule comprising:
(a) a polynucleotide having a base sequence set forth in SEQ ID NO. 1, 3, 5 or 29, or a fragment thereof;
(b) a polynucleotide encoding a polypeptide consisting of an amino acid sequence set forth in SEQ ID NO. 2, 4, 6 or 30, or a fragment thereof;
(c) a polynucleotide encoding a variant polypeptide having an amino acid sequence set forth in SEQ ID NO. 2, 4, 6 or 30, or a fragment thereof, wherein at least one amino acid in the sequence has a mutation selected from the group consisting of substitution, addition, and deletion and wherein the variant polypeptide has biological activity;
(d) a polynucleotide, which is a spliced mutant or alleic mutant of a base sequence set forth in SEQ ID NO. 1, 3, 5 or 29, or a fragment thereof;
(e) a polynucleotide encoding a species homolog of a polypeptide consisting of an amino acid sequence set forth in SEQ ID NO. 2, 4, 6 or 30, or a fragment thereof;
(f) a polynucleotide hybridizable to any one of the polynucleotides of (a) to (e) under stringent conditions and encoding a polypeptide having biological activity; or
(g) a polynucleotide consisting of a base sequence having at least 70% identity to any one of the polynucleotides (a) to (e) or a complementary sequence thereof, and encoding a polypeptide having biological activity, or
(B) a sequence of a promoter portion of the Stm gene.


SEQUENCE LISTING (34 sequences)

SEQ ID NO: 1 - DNA, 2110 bp, Homo sapiens
  CDS (217)..(1131)
  misc_feature (1691)..(1700): n is a, c, g, or t
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagcatccgactgtaaagaatcttcacctatgcctgtgatttgtgggcctgaagaaaactatccatccttgcaaatgtcttctgctgagatgcctcacacggagactgtctctcctcttccttcctccatggatctgcttattcaggacagccctgattcttccaccagtcccaaaggcaaacaacccacttctgcagagaagagtgtcgcaaaaaaggaagacaaggtcccggtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctttactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgccttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcccatccagtcaatctcatggagggtggagtatggttggagcctaatcagcgaggtttctttttttttttttttcctattggatcttcctggagaaaatactttttttttttttttttttgaaacggagtcttgctctgtcgcccaggctggagtgcagtggcgcggtcttggctcactgcaagctccgtctcccgggttcacgccattctcctgcctcagcctcccgagcagctgggactacaggcgcccgccacctcgcccggctaatattttgtatttttagtagagacggggtttcactgtgttagccaggatggtctcgatctcctgaccttgtgatccacccgcctcggcctccctaacagctgggatttacaggcgtgagccaccgcgccctgcctagaaaagacattttaataaccttggctgcnnnnnnnnnngccgtctctggctatagataagtagatctaatactagtttggatatctttagggtttagaatctaacctcaagaataagaaatacaagtacaaattggtgatgaagatgtattcgtattgtttgggattgggaggctttgcttattttttaaaaactattgaggtaaagggttaagctgtaacatacttaattgatttcttaccgtttttggctctgttttgctatatcccctaatttgttggttgtgctaatctttgtagaaagaggtctcgtatttgctgcatcgtaatgacatgagtactgctttagttggtttaagttcaaatgaatgaaacaactatttttcctttagttgattttaccctgatttcaccgagtgtttcaatgagtaaatatacagcttaaacat

SEQ ID NO: 2 - protein, 305 aa, Homo sapiens
MSVDPACPQSLPCFEASDCKESSPMPVICGPEENYPSLQMSSAEMPHTETVSPLPSSMDLLIQDSPDSSTSPKGKQPTSAEKSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 3 - DNA, 2185 bp, Musmusculus
  CDS (191)..(1105)
gagataggctgatttggttggtgtcttgctctttctgtgggaaggctgcggctcacttccttctgacttcttgataattttgcattagacatttaactcttctttctatgatctttccttctagacactgagttttttggttgttgcctaaaaccttttcagaaatcccttccctcgccatcacactgacatgagtgtgggtcttcctggtccccacagtttgcctagttctgaggaagcatcgaattctgggaacgcctcatcaatgcctgcagtttttcatcccgagaactattcttgcttacaagggtctgctactgagatgctctgcacagaggctgcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggttaagacctggtttcaaaaccaaaggatgaagtgcaagcggtggcagaaaaaccagtggttgaagactagcaatggtctgattcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaatgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaatatgagacttacgcaacatctgggcttaaagtcagggcaaagccaggttcctttctttttccaaatattttcatattttttttaaagatttatttattcattatatgtaagtaccctgtagctgtcttcatacactccaaaaaagggcgtcagatcttgttacgtatggttgtgagccaccatgtggttgctgggatttgaactcctgaccttcggaagagcagtcgggtgctcttatccactgagccatctcaccagcccctggtttatttttttaattattatttgctttttgtttatcgagacagggtttctctgcatagctctaattgtctttgaactagctctgcagaccagcctggccttgaactcagagatctgcccacttatctttgcctcctgaatgctgggaccaaaggtggcataccaccacacctggcatatatattgtttatttctatttctatttttattggtgccagagcaaacctaggacttagaacatgctgggcaccaactcaacttctgagctctatttacaacttggtgtgttagtgtatttgtcttagttctgaatttgtcctttttttagtgttaactctaggctttggagacagtgaggtgcatatactctctccttcccaagaataagtgcttgaacacccttacccacgcccacccacccatgctagtcttttttcttagaagcgtgggtcttggtatacactgtgtcattttgaggggtgaggtttaaaagtatatacaaagtataacgatatggtggctactctcgaggatgagacagaaggaccaggagtttgagggtagctcagatatgcaataagttcaaggccaacctgtactatgtttaaatagtaagacagcatctcgataaaataataaaactaaagtctcaacaaaataaaagctttcacctattaaggtgcttgcttgtccttggagtcccccaagagtaactgctatgttaatatctgtagaaagatgtttatatttgactgtaccatgatgaaccgatgccagctggactagtttaaacaaaataaaacactaattttaccttt

SEQ ID NO: 4 - protein, 305 aa, Musmusculus
MSVGLPGPHSLPSSEEASNSGNASSMPAVFHPENYSCLQGSATEMLCTEAASPRPSSEDLPLQGSPDSSTSPKQKLSSPEADKGPEEEENKVLARKQKMRTVFSQAQLCALKDRFQKQKYLSLQQMQELSSILNLSYKQVKTWFQNQRMKCKRWQKNQWLKTSNGLIQKGSAPVEYPSIHCSYPQGYLVNASGSLSMWGSQTWTNPTWSSQTWTNPTWNNQTWTNPTWSSQAWTAQSWNGQPWNAAPLHNFGEDFLQPYVQLQQNFSASDLEVNLEATRESHAHFSTPQALELFLNYSVTPPGEI

SEQ ID NO: 5 - DNA, 1171 bp, Macaca fascicularis
  CDS (218)..(1135)
cattataaatctagagactccaggattttaacgttctgctggactgagctggttgtctcctgttactgtgtaggcgactctctttatcccaacttcttgatacttctgcttctggaggtcatatttctctaacatcttccagaaaagtcttgaagctgccttaaccttttttccagtccacctcttacatttttttctcctcttcctcaatactaacatgagtgtggatccagcttgtccccaaagcttgccttgcttggaagcatccgacagtaaagaatcttcacctatgcctgtgatttgtgggcctgaagaaaactatccatccttgcaaatgtcttctgctgagatgcctcacacggagactgtctctcctcttccttcctccatggatctgcttattcaggacagccccgattcttccaccagtcccaaaggcaaacaacctactgctgcagagaatagtgccacaaaaaaggaagacaaggtcccggtcaagaaacagaaggccagaactgtgttctcttccgcccagctgtgtgtactcaatgatagatttcagagacagaaatacctcagcctccagcagatgcaagaactttccaacatcctgaacctcagctacaaacaggtgaagacctggttccagaaccagagaatgaaatctaagaggtggcagaaaaacaactggccaaagaatagcaatggtgtgactcagaaggcctcagcacctacctaccccagcctctactcttcctgccaccagggatgcctggtaaacccgactgggaaccttccaatgtggagcaaccagacctggaacaattcatcctggagcaaccagacccagaacatccagtcctggagcaaccactcctggaacgctcagacctggtgcacccagtcctggaacaatcaggcctggaacagtcccttctctaactgtggagaggaatctctgcagtcctgcttgcagttccagccaaattctcctgccagtgacttggaggctgccttggaagctgctggggaaggccttaatgtaatacagcagacgactaggtatttgagtactccacaaactgtggatttactcctaaactactccacgaacatgcaacctgaagatgtgtgaagatgagtgaaaatgatattactcaatttcagtctg

SEQ ID NO: 6 - protein, 305 aa, Macaca fascicularis
MSVDPACPQSLPCLEASDSKESSPMPVICGPEENYPSLQMSSAEMPHTETVSPLPSSMDLLIQDSPDSSTSPKGKQPTAAENSATKKEDKVPVKKQKARTVFSSAQLCVLNDRFQRQKYLSLQQMQELSNILNLSYKQVKTWFQNQRMKSKRWQKNNWPKNSNGVTQKASAPTYPSLYSSCHQGCLVNPTGNLPMWSNQTWNNSSWSNQTQNIQSWSNHSWNAQTWCTQSWNNQAWNSPFSNCGEESLQSCLQFQPNSPASDLEAALEAAGEGLNVIQQTTRYLSTPQTVDLLLNYSTNMQPEDV

SEQ ID NO: 7 - DNA, 2114 bp, Homo sapiens
  CDS (217)..(1134)
attataaatctagagactccaggattttaacgttctgctggactgagctggttgcctcatgttattatgcaggcaactcactttatcccaatttcttgatacttttccttctggaggtcctatttctctaacatcttccagaaaagtcttaaagctgccttaaccttttttccagtccacctcttaaattttttcctcctcttcctctatactaacatgagtgtggatccagcttgtccccaaagcttgccttgctttgaagaatccgactgtaaagaatcttcacctatgcctgtgatttgtgggcctgaagaaaactatccatccttgcaaatgtcttctgctgagatgcctcacacggagactgtctctcctcttccctcctccatggatctgcttattcaggacagccctgattcttccaccagtcccaaaggcaaacaacccacttctgcagagaatagtgtcgcaaaaaaggaagacaaggtcccagtcaagaaacagaagaccagaactgtgttctcttccacccagctgtgtgtactcaatgatagatttcagagacagaaatacctcagcctccagcagatgcaagaactctccaacatcctgaacctcagctacaaacaggtgaagacctggttccagaaccagagaatgaaatctaagaggtggcagaaaaacaactggccgaagaatagcaatggtgtgacgcagaaggcctcagcacctacctaccccagcctctactcttcctaccaccagggatgcctggtgaacccgactgggaaccttccaatgtggagcaaccagacctggaacaattcaacctggagcaaccagacccagaacatccagtcctggagcaaccactcctggaacactcagacctggtgcacccaatcctggaacaatcaggcctggaacagtcccttctataactgtggagaggaatctctgcagtcctgcatgcagttccagccaaattctcctgccagtgacttggaggctgctttggaagctgctggggaaggccttaatgtaatacagcagaccactaggtattttagtactccacaaaccatggatttattcctaaactactccatgaacatgcaacctgaagacgtgtgaagatgagtgaaactgatattactcaatttcagtctggacactggctgaatccttcctctcccctcctcccatccctcataggatttttcttgtttggaaaccacgtgttctggtttccatgatgcctatccagtcaatctcatggagggtggagtatggttggagcctaatcagcgaggtttctttttttttttttcctattggatcttcctggagaaaatacttttttttttttttttgagacggagtcttgctctgtcgcccaggctggagtgcagtggcgcggtcttggctcactgcaagctccgcctcccgggttcacgccattctcctgcctcagcctcccgagcagctgggactacaggcgcccgccacctcgcccggctaatattttgtatttttagtagagacagggtttcactgtgttagccaggatggtctcgatctcctgaccttgtgatccgcccgcctcggcctccctaacagctgggattacaggcgtgagccaccgcgccctgcctagaaaagacattttaataaccttggctgctaaggacaacattgatagaagccgtctctggctatagataagtagatctaatactagtttggatatctttagggtttagaatctaacctcaagaataagaaatacaagtacgaattggtgatgaagatgtattcgtattgtttgggattgggaggctttgcttatttttttaaaactattgaggtaaagggttaagctgtaacatacttaattgatttcttaccgtttttggctctgttttgctatatcccctaatttgttggttgtgctaatctttgtagaaagaggtcttgtatttgctgcatcgtaatgacatgagtactactttagttggtttaagttcaaatgaatgaaacaaatatttttcctttagttgattttaccctgatttcaccgagtgtttcgatgagtaaatatacagcttaaacat

SEQ ID NO: 8 - protein, 305 aa, Homo sapiens
MSVDPACPQSLPCFEESDCKESSPMPVICGPEENYPSLQMSSAEMPHTETVSPLPSSMDLLIQDSPDSSTSPKGKQPTSAENSVAKKEDKVPVKKQKTRTVFSSTQLCVLNDRFQRQKYLSLQQMQELSNILNLSYKQVKTWFQNQRMKSKRWQKNNWPKNSNGVTQKASAPTYPSLYSSYHQGCLVNPTGNLPMWSNQTWNNSTWSNQTQNIQSWSNHSWNTQTWCTQSWNNQAWNSPFYNCGEESLQSCMQFQPNSPASDLEAALEAAGEGLNVIQQTTRYFSTPQTMDLFLNYSMNMQPEDV

SEQ ID NO: 9 - DNA, 2080 bp, Mus musculus
  CDS (84)..(1001)
agagataggctgatttgaccctgagttttttggttgttgcctaaaaccttttcagaaatcccttccctcgtcatcacactgacatgagtgtgggtcttcctggtccccacagttggcctagttctgaggaagcatcgaattctggggacgcctcatcaatgcctgcagtttttcatcccgagaactattcttgcttacaagggtctgctactgagatgctctgcacagaggctgcctctcctcgcccttcctctgaagacctgcctcttcaaggcagccctgattcttctaccagtcccaaacaaaagctctcaagtcctgaggctgacaagggccctgaggaggaggagaacaaggtccttgccaggaagcagaagatgcggactgtgttctctcaggcccagctgtgtgcactcaaggacaggtttcagaagcagaagtacctcagcctccagcagatgcaagaactctcctccattctgaacctgagctataagcaggttaagacctggtttcaaaaccaaaggatgaagtgcaagcggtggcagaaaaaccagtggttgaagactagcaatggtctgattcagaagggctcagcaccagtggagtatcccagcatccattgcagctatccccagggctatctggtgaacgcatctggaagcctttccatgtggggcagccagacttggaccaacccaacttggagcagccagacctggaccaacccaacttggaacaaccagacctggaccaacccaacttggagcagccaggcctggaccgctcagtcctggaacggccagccttggaacgctgctccgctccataacttcggggaggactttctgcagccttacgtacagttgcagcaaaacttctctgccagtgatttggaggtgaatttggaagccactagggaaagccatgcgcattttagcaccccacaagccttggaattattcctgaactactctgtgactccaccaggtgaaatatgagacttacgcaacatctgggcttaaagtcagggcaaggccaggttccttccttcttccaaagattttcatatttttttaaaagatttatttattcattatatgtaagtacactgtagctgtcttcagacactccagaagagggcgtcagatcttgttgcggatggttgtgagccaccatgtggttgctgggatttgaactctggaccttcggaagagcagtcgggtgctcttatccactgagccatctcaccagcccctggtttatttttttaattattatttgctttttgtttatcaagacagggtttctctgcatagctctaattgtctttgaactagctctgcagaccagctagcctcaaactcagagatctgcccacttatctttgcctcctgaatgctgggaccaaaggtggcataccaccacacctggcatatatattgtttatttctatttctatttttattggtgccagagcaaacctaggacttagaacatgctgggcaccaactcaacttctgagctctatttacaactcggtgtgttagtgtatttgtcttagttctgaatttgtcctttttttagtgttaactctaggctttggagacagtgagttgcatatactctctccttcccaagaataagtgcttgaacacccttacccacgcccacccacccacgctagtctttttttcttaagaagcgtgggtcttggtatacactgtgtgtcattttgaggggtgaggtttaaaagtatatacaaagtataacgatatggtggctactctcgaggatgagacagaaggaccaggagtttgagggtagctcggatatgcaataagttcaaggccaacctgtactatgtttaaatagtaagacagcatcttgataaaataataaaactaaagtctcaacaaaataaaagctttcacctgttaaggtgcttgcttgtccttggagtcccccaagagtaactgctatgttaatatctgtagaaagatgtttatatttgactgtaccatgatgaaccgatgccagctggactagtttaaacaaaataaaacactaatttaaaaat

SEQ ID NO: 10 - protein, 305 aa, Mus musculus
MSVGLPGPHSWPSSEEASNSGDASSMPAVFHPENYSCLQGSATEMLCTEAASPRPSSEDLPLQGSPDSSTSPKQKLSSPEADKGPEEEENKVLARKQKMRTVFSQAQLCALKDRFQKQKYLSLQQMQELSSILNLSYKQVKTWFQNQRMKCKRWQKNQWLKTSNGLIQKGSAPVEYPSIHCSYPQGYLVNASGSLSMWGSQTWTNPTWSSQTWTNPTWNNQTWTNPTWSSQAWTAQSWNGQPWNAAPLHNFGEDFLQPYVQLQQNFSASDLEVNLEATRESHAHFSTPQALELFLNYSVTPPGEI

SEQ ID NO: 11 - DNA, 20 bp, Mus musculus
  F1 primer
gcgcattttagcaccccaca

SEQ ID NO: 12 - DNA, 20 bp, Mus musculus
  R1 primer
gttctaagtcctaggtttgc

SEQ ID NO: 13 - DNA, 20 bp, Mus musculus
  F2 primer
gaattctgggaacgcctcat

SEQ ID NO: 14 - DNA, 20 bp, Mus musculus
  R2 primer
ccagatgttgcgtaagtctc

SEQ ID NO: 15 - DNA, 24 bp, Mus musculus
  Oct4RT/1 primer
ggcgttctctttggaaaggtgttc

SEQ ID NO: 16 - DNA, 20 bp, Mus musculus
  Oct4RT/2 primer
ctcgaaccacatccttctct

SEQ ID NO: 17 - DNA, 26 bp, Mus musculus
  G3PDH-5 primer
tgaaggtcggtgtcaacggatttggc

SEQ ID NO: 18 - DNA, 24 bp, Mus musculus
  G3PDH-3 primer
catgtaggccatgaggtccaccac

SEQ ID NO: 19 - DNA, 28 bp, Mus musculus
  Stm-f primer
cgggatccatgagtgtgggtcttcctgg

SEQ ID NO: 20 - DNA, 29 bp, Mus musculus
  Stm-r primer
tcccccgggtcatatttcacctggtggag

SEQ ID NO: 21 - DNA, 18 bp, Mus musculus
  exon2F primer
cctctcctcgcccttcct

SEQ ID NO: 22 - DNA, 21 bp, Mus musculus
  exon2R primer
ctgcttatagctcaggttcag

SEQ ID NO: 23 - DNA, 21 bp, Mus musculus
  Ex3F primer
gtggttgaagactagcaatgg

SEQ ID NO: 24 - DNA, 20 bp, Mus musculus
  Int3F primer
ctatggctgttgggtatgga

SEQ ID NO: 25 - DNA, 20 bp, Mus musculus
  F3 primer
ctttgaactagctctgcaga

SEQ ID NO: 26 - DNA, 21 bp, Mus musculus
  R3 primer
tgaacttattgcatatctgag

SEQ ID NO: 27 - DNA, 19 bp, Mus musculus
  F4 primer
cagggctatctggtgaacg

SEQ ID NO: 28 - DNA, 19 bp, Mus musculus
  R4 primer
gagcacccgactgctcttc

SEQ ID NO: 29 - DNA, 939 bp, Rattus rattus
  CDS (1)..(939)
atgagcgtggatctttctggtccccacagtctgcctagttgtgaggaagcatcgaactctggggattcctcgccgatgcctgccgttcatcttcctgaggaaaattattcttgcttacaagtgtctgctactgagatgctctgcacagagactgcctctcctccgccttcctctggggacctacctcttcaagatagccctgattcttctagcaatcccaagctaaagctgtctggtcccgaggctgacgagggccctgagaagaaagaagagaacaaggtcctcaccaagaagcagaagatgcggactgtgttctctcaggcccagttgtgtgcactcaaggataggtttcagaggcaaaggtacctcagcctccagcagatgcaagatctctctaccattctgaacctgagctataagcaggtgaagacctggttccaaaaccaaagaatgaagtgcaagaggtggcagaaaaaccaatggttgaagactagcaacggcctgactcagaagggctcagcgccggtggagtatcccagcatccattgcagctattctcagggctatctgatgaacgcgtctggaaaccttccagtatggggcagtcagacctggaccaacccaacttggaacaaccagacctggaccaacccaacctggagcaaccagacctggaccaacccaacttggagcaaccaggcctggagcactcagtcctggtgtactcaggcctggaacagccagacttggaacgctgctccgctccataacttcggggaggactccctgcagccttatgtgccgttgcagcaaaacttctccgccagtgatttggaggcgaatttggaagccactagggaaagccaggcgcattttagtaccccgcaagccttggaattgttcctgaactactccgtgaattctccaggcgaaatatga

SEQ ID NO: 30 - protein, 312 aa, Rattus rattus
MSVDLSGPHSLPSCEEASNSGDSSPMPAVHLPEENYSCLQVSATEMLCTETASPPPSSGDLPLQDSPDSSSNPKLKLSGPEADEGPEKKEENKVLTKKQKMRTVFSQAQLCALKDRFQRQRYLSLQQMQDLSTILNLSYKQVKTWFQNQRMKCKRWQKNQWLKTSNGLTQKGSAPVEYPSIHCSYSQGYLMNASGNLPVWGSQTWTNPTWNNQTWTNPTWSNQTWTNPTWSNQAWSTQSWCTQAWNSQTWNAAPLHNFGEDSLQPYVPLQQNFSASDLEANLEATRESQAHFSTPQALELFLNYSVNSPGEI

SEQ ID NO: 31 - DNA, 384 bp, Homo sapiens
ttaaaaatattaaagttttatcccattcctgttgaaccatattcctgatttaaaagttggaaacgtggtgaacctagaagtatttgttgctgggtttgtcttcaggttctgttgctcggttttctagttccccacctagtctgggttactctgcagctacttttgcattacaatggccttggtgagactggtagacgggattaactgagaattcacaagggtgggtcagtagggggtgtgcccgccaggaggggtgggtctaaggtgatagagccttcattataaatctagagactccaggattttaacgttctgctggactgagctggttgcctcatgttattatgcaggcaactcactttatcccaatttcttgatactttt

SEQ ID NO: 32 - DNA, 382 bp, Mus musculus
gagatcgccagggtctggaggtgcagccgtggttaaaagatgaataaagtgaaatgaggtaaagcctctttttgggggggggggatgtctttagatcagaggatgccccctaagctttccctccctcccagtctgggtcaccttacagcttcttttgcattacaatgtccatggtggaccctgcaggtgggattaactgtgaattcacagggctggtggggcgtgggtgccgcctgggtgcctgggagaatagggggtgggtagggtaggaggcttgaggggggaggagcaggacctaccctttaaatctatcgccttgagccgttggccttcagataggctgatttggttggtgtcttgctctttctgtgggaaggctgcg

SEQ ID NO: 33 - DNA, 382 bp, Macaca fascicularis
tctttatttttaaagttttatcgcattcctgttgaaccattcctgatttaaaagttggaaacgtgatgaacctggaagtatttattgctgggtttgtcttcaggttctgttacccggttttctagttcccgacctcgtctgggttactccacagttacttttgcattacaatggccttggtgagactggcagacgggattaactgagaattcacaagggtgggccggtgggggtgtgcccaccaggaggggtgggtctaagggtgatagagccttcattataaatctagagactccaggattttaacgttctgctggactgagctggttgtctcctgttactgtgtaggcgactctctttatcccaacttcttgatacttct

SEQ ID NO: 34 - DNA, 2394 bp, Mus musculus
gaagagagagagagagagagtggtgtaaacagtgggtctgaagccttcgagggaggagggcaagatgccgcgctctgcgtttctccagctggatgtggacggaggtcaaccagccacattagtttatgtctaaaagtgcctaattgaaacaagaaatggctgctttagccgggtgtggtggcacacgcccttaatcccagcactcaggaggcagaggcaggcagatttctgagttcaaggccagcctggtctacagagtgagttccaggacagccagagctacacagagaaaccctgtctcgaaaaaccaaccaaccaaccaaccaaacaaacaaaaaaggaaatggctggtttaattatatcacactgttcatcagaataaagagaaaaatctcaaaaaaaaaaaaaaaaaaaaagaggttcagtcaggctgggcaatggaggcagtagtggccgtggtggtcttggcagcagtggaggccttggtggcagtggtgacagacagtgatagtgtggcagcagttgaggcagtggtggtcgtagtggcagtgatggcattggtggcagggttggtgtggctcagaggtggcagtggtggcagtggaggcattgcaggctttagtggcaatggtagtggggcagcagtggtagccatggtggcacagtggtagcaacggtggtagtggcagccgtggtggccgcagaggcagtggtaacaatggtggcttggtggcagtgctgacagtggtagtattggtagcattggtggcagtggaagaagggaagtggtggtaatggtggcaggggtggcagcagaggcagacagtggtgacgatggtggcagtggtgctgtggcacagtggtggcattggtggcatggcacagtggtggcaatgatggtgtggtgcagtggtagcagtggagacagtggtggcagtggagacagtagtagtatggtggctgtggtggccatggcggcagtggtggcacatacaggcctttaatgccagcacatgggaggcagaggcagttggctctctgtgagttggaagccagcttgttctacagagcaagtcccaggacagccagggctacacagagaaaccctgcctagaataataacaaaactcaaacaaacaccaccaacccccgcgctccgccccgccccccccaaaaaaaaccccaaaccaaaaagagccattcaagcttagtcttttgtagaataaaacccaggaagaaccactcctaccaatactcactgtggtagagtcttcacatgggagatgttacgagaggacggcccttccctctctgcttatacacagaagccgacttaagctgggttagagtgctttcactcacttatctgtgagcacaaggactgatcggcaaactttgaacttgggatgtggaaataggaagatcaggagtttgggaccagctagagcaatagactgtgaggccagtctcgggtacgtgaaactctatctcaaagaaatgaccaaaaccaaatttgaaatacgataaatttcttcttccattgcttagacggctgaggcacttgggattgcagtaagtctgaagccagcctaggttttacagtgagaacttgtctcaaaacaaaacagaaggcctctacattaatttaaacactccttaaattgggcatggtggtagacaagcctggtctacatactgagtataagctactcaaggcaacagagaaaaacctgtctcaaaaccaaagcatggaccaacttactaaggtagcccgagtcttaagcaggacacaggctctttcttcagacttgcgttaaaaagccgcacttttggagggaagatttccccaggtttcccaatgtgaagagcaagcaagaaacgctgagtgctgaaaggaaagccgtgtataaacagagactttggcagcaaggtctgactctttcatgtctgtagaaagaatggaagaggaaactcagatcccccacttgacctgaaacttcccactagagatcgccagggtctggaggtgcagccgtggttaaaatgatgaataaagtgaaatgaggttaagcctctttttggggggggggggggaagtgtctttagatcagaggatgccccctaagctttccctccctcccagtctgggtcaccttacagcttcttttgcattacaatgtccatggtggaccctgcaggtgggattaactgtgaattcacagggctggtggggcgtgggtgccgcctgggtgcctgggagaatagggggtgggtagggtaggaggcttgaggggggaggagcaggacctaccctttaaatctatcgccttgagccgttggccttcagataggctgatttggttggtgtcttgctctttctgtgggaaggctgcg